(19) United States
(12) Patent Application Publication
(10) Pub. No.: US 2010/0074897 A1
(43) Pub. Date: Mar. 25, 2010

(54) Methods and Compositions related to HIF-1 alpha

(75) Inventor: Eric L. Huang, North Salt Lake, UT (US)

Correspondence Address: Ballard Spahr LLP, SUITE 1000, 999 PEACHTREE STREET, ATLANTA, GA, 30309-3915, US

(73) Assignee: University of Utah Research Foundation, Salt Lake City, UT (US)

(21) Appl. No.: 12/517,132

(22) PCT Filed: Dec. 3, 2007
(86) PCT No.: PCT/US2007/086264

§ 371 Date: Jul. 10, 2009

Related U.S. Application Data
(60) Provisional application No. 60/868,188, filed on Dec. 1, 2006.

(51) Int. Cl. A61K 39/395; A61K 31/7105; G01N 33/53; A61P 35/00

(52) U.S. Cl. 424/133.1; 514/44 R; 514/44 A; 424/130.1; 424/141.1; 436/94

(57) ABSTRACT

Disclosed are compositions and methods related to HIF-1α.

### CROSS-RELATED APPLICATIONS

This application claims the benefit of U.S. provisional application No. 60/868,188, filed Dec. 1, 2006. The aforementioned application is herein incorporated by this reference in its entirety.

### STATEMENT REGARDING FEDERALLY SPONSORED RESEARCH

This invention was made with government support under Grant 1K22 CA084563-01 awarded by the National Cancer Institute. The government has certain rights in the invention.

### BACKGROUND

Ischemic heart disease occurs when the heart muscle does not receive an adequate blood supply and is thus deprived of necessary levels of oxygen and nutrients. Ischemia is commonly a result of atherosclerosis which causes blockages in the coronary arteries that provide blood flow to the heart muscle.

Ischemic heart disease can result in certain adaptive responses within the heart which are likely to be beneficial. Among these-responses are: 1) increased expression of angiogenic growth factors and their receptors, leading to the formation of collateral circulation around blocked coronary arteries; 2) increased expression of glycolytic enzymes as a means to activate a metabolic pathway which does not require oxygen; and 3) expression of heat shock proteins which can protect the ischemic tissue from death.

At least some of these responses appear to be regulated by a complex oxygen sensing mechanism which eventually leads to the activation of transcription factors which control the expression of critical genes involved in this adaptation. Because this altered gene expression occurs only in response to hypoxia, which usually only occurs when a strain such as exercise is placed upon the diseased heart, cardiac patients do not usually receive much benefit from this endogenous compensatory mechanism. As a result, a number of conventional therapies attempt to supplement the natural therapeutic responses of the heart to ischemia.

For example, such treatments include pharmacological therapies, coronary artery bypass surgery and percutaneous revascularization using techniques such as balloon angioplasty. Standard pharmacological therapy is predicated on strategies that involve either increasing blood supply to the heart muscle or decreasing the demand of the heart muscle for oxygen and nutrients.

Increased blood supply to the myocardium is achieved by agents such as calcium channel blockers or nitroglycerin. These agents are thought to increase the diameter of diseased arteries by causing relaxation of the smooth muscle in the arterial walls. Decreased demand of the heart muscle for oxygen and nutrients is accomplished either by agents that decrease the hemodynamic load on the heart, such as arterial vasodilators, or those that decrease the contractile response of the heart to a given hemodynamic load, such as beta-adrenergic receptor antagonists.

Surgical treatment of ischemic heart disease is based on the bypass of diseased arterial segments with strategically placed bypass grafts (usually saphenous vein or internal mammary artery grafts). Percutaneous revascularization is based on the use of catheters to reduce the narrowing in diseased coronary arteries. All of these strategies are used to decrease the number of, or to eradicate ischemic episodes, but all have various limitations.

More recently, delivery of angiogenic factors or heat shock proteins via protein or gene therapy has been proposed to further augment the heart's natural response to ischemia. Indeed, various publications have discussed the uses of gene transfer for the treatment or prevention heart disease. See, for example, Mazur et al.,“Coronary Restenosis and Gene Therapy”, Molecular and Cellular Pharmacology 21:104-111 (1994); French, B. A. “Gene Transfer and Cardiovascular Disorders” Herz. 18(4):222-229 (1993); Williams, “Prospects for Gene Therapy of Ischemic Heart Disease”, Am. J. Med. Sci. 306:129-136 (1993); Schneider and French “The Advent of Adenovirus: Gene Therapy for Cardiovascular Disease” Circulation 88:1937-42 (1993). International Patent Application No. PCT/US93/11133, entitled “Adenovirus-Mediated Gene Transfer to Cardiac and Vascular Smooth Muscle” reporting the use of adenovirus-mediated gene transfer for regulating function in cardiac vascular smooth muscle.

Cancer is essentially a genetic disease (Vogelstein and Kinzler, 2004). As a result of gene mutations, activation of oncogenes and inactivation of tumor-suppressor genes collaborate on the neoplastic process by stimulating cell proliferation, or inhibiting cell death or cell-cycle arrest. Furthermore, hereditary inactivation of stability genes or caretakers, mainly involved in DNA repair, gives rise to genetic alterations leading to tumorigenesis. Genetic instability is a hallmark of most human cancers, arising from changes at the nucleotide and the chromosomal levels (Lengauer et al, 1998).

The primary structure of DNA is constantly subjected to alteration by cellular metabolites and exogenous DNA-damaging agents. DNA repair is essential to safeguard the genetic integrity by correcting mutations arising from myriad types of damage (Friedberg, 2003). Multiple distinct mechanisms of excision repair have been identified, including nucleotide excision repair, base excision repair, and mismatch repair. Apart from coping with damaged bases or replication errors, cells frequently suffer breakage of the DNA duplex. DNA double-strand breaks (DSBs) arise primarily from stalled replication forks, and genetically programmed processes in developing lymphocytes, and exogenous factors such as ionizing radiation. Proper DSB repair is fundamental to the prevention of chromosome loss, translocations, and truncations.

NBS1/nibrin is part of the evolutionarily conserved MRE11A-RAD50NBS1 (MRN) complex, which interacts with DSBs early in the DNA damage response (Carney et al, 1998; D'Amours and Jackson, 2002). The genetic defect in NBS1 gene, stemming predominantly from a 5-base-pair deletion in exon 6, is responsible for the Nijmegan breakage syndrome, a hereditary disorder characterized by chromosomal instability and a predisposition to malignancies (Varon et al, 1998). Cells derived from NBS patients show high sensitivity to ionizing radiation, chromosome fragility, accelerated shortening of telomeres, and deficiency in cell-cycle checkpoints (D'Amours and Jackson, 2002). It has been shown that Nbs1 knockout mice manifested increased chromosomal breaks, owing to reduced gene conversion and sister chromatid exchanges (Tauchi et al, 2002; Frappart et al, 2005). At the molecular level, NBS1 also interacts with γ-H2AX, a phosphorylated histone H2AX detected at the sites of nascent DSBs (Rogakou et al, 1998; Paull et al, 2000), for relocating MRE11A-RAD50 to the vicinity of DNA damage (Kobayashi et al, 2002).

Although deficiency in DNA repair arising from germline mutations has been linked to various hereditary cancers, no somatic mutation in DNA repair genes has been observed in the majority of sporadic cancers, showing functional impairment of DNA repair in these cancers. Numerous studies have indicated that the tumor microenvironment, characterized by hypoxia, low pH, and nutrient deprivation, promotes genetic instability and tumor progression (Bindra and Glazer, 2005). Hypoxia has been shown to induce chromosomal fragility and polyploidy in cell-culture and animal models (Coquelle et al, 1998; Nelson et al, 2004). It has also been shown that hypoxia inhibits DNA repair by down-regulating genes involved in mismatch repair and homologous recombination (Mihaylova et al, 2003; Bindra et al, 2004; Bindra et al, 2005; Koshiji et al, 2005).

Accordingly, there exists a need in the art for compositions and methods for regulating cell-cycle arrest and genetic instability, two major obstacles to cancer and ischemic disease treatment.

### SUMMARY

Disclosed herein is a nucleic acid molecule encoding a polypeptide comprising PAS-B of a hypoxia inducible factor, wherein the PAS-B comprises at least one mutation which differs from naturally occurring PAS-B of a hypoxia inducible factor

Also disclosed are expression vectors comprising a nucleic acid molecule operatively linked to an expression control sequence.

Disclosed herein is a method for increasing the expression in a target cell of a hypoxia-inducible gene, said method comprising the steps of (a) introducing into said cell an expression vector as disclosed herein; and (b) allowing expression of said protein encoded by said expression vector.

Also disclosed is a method for providing sustained expression of biologically active HIF-1α in a cell under normoxic conditions, said method comprising the step of introducing into said cell a nucleic acid molecule according to those disclosed herein, operatively linked to an expression control sequence which directs its expression in said cell.

Also disclosed is a method for reducing ischemic tissue damage in a subject having a hypoxia-associated disorder comprising the steps of administering to said subject an effective amount of a pharmaceutical composition.

Also disclosed is a method for reducing ischemic tissue damage in a subject having a hypoxia-associated disorder comprising the steps of: (a) isolating cells to be implanted into said subject (b) introducing into said cells an expression vector as disclosed herein; and (c) implanting said cells containing said expression vector into said subject.

Disclosed is a method of treating cancer in a subject in need thereof, comprising administering an effective amount of a HIF-1α PAS-B inhibitor or a mutant PAS-B, wherein the cancer is a HIF-1α expressing cancer.

Also disclosed is a method of inhibiting the growth of a solid hypoxic tumor in a subject, comprising administering an effective amount of an HIF-1α PAS-B inhibitor or a mutant PAS-B.

Disclosed is a method of screening for a test compound that modulates HIF-1α PAS-B, comprising contacting HIF-1α PAS-B with a test compound; detecting interaction between HIF-1α PAS-B and the test compound; wherein interaction between HIF-1α PAS-B and the test compound indicates a test compound that modulates HIF-1α PAS-B.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows distinct roles of HIF-1α and HIF-2α in mediating NBS1 repression by hypoxia. (A) HCT116 and HCT116 TP53^{-/-} cells, transfected with siRNA targeting HIF1A or EPAS1 (encoding HIF-2α) were subjected to normoxic (N) or hypoxic (H) treatment (1% O₂, 16 h). Mock transfection (−) and luciferase siRNA (luc) were used as negative controls. NBS1, MSH2, and PGK1 mRNA levels were determined with quantitative real-time PCR. Representative results from three independent experiments in triplicate are presented as means+standard errors. (B) U-2 OS cells were treated as in (A) and assayed for specific protein levels by sequential probing of the same blot with the corresponding antibodies. (C) HCT116 cells were infected with adenoviruses expressing HIF-1αΔODD, HIF-1αΔODD (LCLL), HIF-1α (1-329), and HIF-2α for 16 h. Cells with treatment (−) or infected with Ad-GFP served as controls. NBS1 and PGK1 mRNA levels were determined with real-time PCR. (D) U-2 OS cells were infected with the adenoviruses as above and examined with real-time PCR. An adenovirus expressing an N-terminal HIF-1α lacking the PAS-B domain, HIF-1α (1-167), was also included.

FIG. 2 shows the N-terminal HIF-1α is sufficient to induce DNA DSB. U-2 OS cells were infected with adenoviruses expressing HIF-1α variants as indicated, and stained by immunofluorescence with antibodies against γ-H2AX (red) and 53BP1 (green). Representative fields are presented together with merged images. All of the HIF-1α variants, with the exception of Ad-HIF-1α (1-167), significantly increased the number of colocalized γ-H2AX and 53BP1 foci.

FIG. 3 shows selective Myc displacement in NBS1 gene under hypoxia. (A) A schematic representation of part of the human NBS1 is shown from nucleotides 3400-5900 of gene locus AB013139. The hatched box depicts 5′UTR and exon 1, and the gray box part of intron 1. The black bar specifies an E-box in the intron. Predicted Sp1 sites are in gray bars in the promoter. Four sets of PCR primers, designated as P1, P2, P3, and In1, were used for amplification of the NBS1 regulatory regions, as marked with double-arrow lines. (B) Chromatin immunoprecipitations were performed with normoxic (N) and hypoxic (H) U-2 OS cells. Antibodies used are against RNA polymerase II (Pol II), Sp1, Myc, HIF-1α, and p53. Normal immunoglobulin (IgG) served as a negative control, whereas the Pol II antibody served as a positive control. Sheared genomic DNA before immunoprecipitations was included as control of input DNA. The same immunoprecipitates were subjected to PCR amplification with four sets of NBS1 primers and one set of MSH2 primers (Koshiji et al, 2005). Myc displacement occurred in P2 but not in intron 1.

FIG. 4 shows HIF-1α PAS-B differs from HIF-2α PAS-B in Sp1 binding. (A) A schematic representation of HIF-1α and its deletion mutants. Structural domains of HIF-1α (Huang and Bunn, 2003) are indicated at the top, and the corresponding residues on the left. Sp1-binding activity of each mutant is summarized. (B) The deletion mutants of N-terminal HIF-1α, as indicated, were translated in rabbit reticulocyte lysate with [³⁵S]methionine and subjected to anti-Sp1 immunoprecipitations (α-Sp1). Input, 10% of lysates before immunoprecipitations. (C) HIF-1αΔODD (ΔODD), PAS1B, PAS1B T327P (T327P), and Myc were translated in vitro as above. Equal amount of the translated products were mixed as denoted and subjected to anti-Sp1 immunoprecipitations to determine their competitiveness for Sp1 binding. Molecular weight markers are indicated. (D, E) HIF-1αΔODD, PAS1B, HIF-2α, and PAS2B were translated in vitro as above and subjected to anti-Sp1 immunoprecipitations (D), or transfected into HeLa cells and immunoprecipitated with an anti-Sp1 antibody, followed by immunoblotting (IP-IB) with respective antibodies against specific proteins as indicated (E). To block PAS-B proteolysis, cells were treated with Cbz-LLL for 4 h before harvest. Input, 10% of total whole-cell extract subjected to direct immunoblotting. Arrowhead denotes nonspecific detection. (F) In vitro translated PAS1B was immunoprecipitated with anti-Sp1 antibody in the presence of unlabeled PAS1B or its T327P mutant. Addition of increasing amounts (10, 100, and 1000 pmol) of synthetic peptide corresponding to HIF-1α residues 299-329 competed with PAS1B for Sp1 binding.

FIG. 5 shows HIF-2α Pro-329 precludes Sp1 binding. (A) Sequence alignment of HIF-2α residues 301-331 with those of HIF-1α. A predicted phosphorylation motif at Thr-324 by PKD1 is underlined. Both HIF-2α Thr-324 and HIF-1α Thr-322 are specified. Shaded residues are unique in HIF-2α and critical for the phosphorylation motif. (B, C) T301V, V317L, and T327P mutants in the context of PAS1B (B) and HIF-1αΔODD (C) were translated in vitro as above and subjected to anti-Sp1 immunoprecipitations. (D) Wild-type (WT) HIF-2α and PAS2B, and their P329T mutants were analyzed as in (B). (E, F) P329T mutant in the context of PAS2B (E) and HIF-2α (F) were transfected into HeLa cells. Anti-Sp1 immunoprecipitations were performed, followed by immunoblotting to detect the co-immunoprecipitates as indicated.

FIG. 6 shows HIF-2α Thr-324 is phosphorylated by PKD1, thereby abrogating Sp1 binding. (A) PAS1B and its T327P mutant were translated in vitro in rabbit reticulocyte lysate (R) or in wheat germ extract (W). Their interactions with Sp1 were determined by anti-Sp1 immunoprecipitations. (B) PAS2B and its P329T mutant were analyzed as in (A). (C) PAS2B and its T324V mutant, as well as PAS1B were transfected into U-2 OS cells, and labeled with ³²P-orthophosphate for 4 h. The expressed proteins were immunoprecipitated with anti-FLAG antibodies. The proteasome inhibitor Cbz-LLL was added (+) to ensure adequate PAS1B expression. (D) PAS-B variants, produced from rabbit reticulocyte lysate or wheat germ extract as indicated, were subjected to treatment with λprotein phosphatase (λPPase, 100 U for 30 min) before anti-Sp1 immunoprecipitations. (E) A synthetic peptide of HIF-2α containing the predicted PKD1 motif was used as a substrate of recombinant PKD1 in an in vitro kinase assay. Two additional synthetic peptides, one harboring mutation at HIF-2α Thr-324 and the other a HIF-1α equivalent, were included as controls. PKD1-mediated phosphorylation was presented in counts per minute (c.p.m.). (F) PAS1B and PAS2B were translated in rabbit reticulocyte lysate in the presence of resveratrol (Res, 100 μM). Their Sp1-binding activity was analyzed as above. (G) HeLa cells expressing PAS1B and PAS2B were treated with 5 nM of resveratrol. Anti-Sp1 immunoprecipitations were performed, followed by immunoblotting (IP-IB) as indicated. Cells were treated with Cbz-LLL for 4 h before harvest to increase the PAS-B protein levels. (H) Synthetic peptides containing a PKD1 consensus sequence (Subs) and the predicted PKD1 site in HIF-2α were tested, respectively, for phosphorylation by recombinant PKD1 in the presence or absence of resveratrol in an in vitro kinase assay. Recombinant PKD1 was preincubated with resveratrol (100 μM) for 10 min. (I) Immunoprecipitated endogenous PKD1 was used for in vitro kinase assays with synthetic peptides as indicated in the absence or presence of resveratrol.

FIG. 7 shows nonphosphorylated PAS-B induces NBS1 repression and DNA DSBs. (A) PAS1B, PAS2B, and their mutants, as indicated, were translated in rabbit reticulocyte lysate and subjected to anti-Sp1 immunoprecipitations. (B) The PAS-B variants were expressed transiently in U-2 OS cells. Endogenous NBS1 and the transfected PAS-B were determined by sequential probing of the same blot with the corresponding antibodies, as indicated. (C) γ-H2AX and 53BP1 foci were determined by immunofluorescence as in FIG. 2 in U-2 OS expressing the PAS-B variants. PAS1B and PAS2B T324V expression significantly increased γ-H2AX foci, as pointed by arrowheads. (D) The PAS-B variants were transfected into HCT116 and HCT116 TP53^{-/-} cells. NBS1, MSH2 and PGK1 mRNA levels were determined with real-time PCR. Representative results are shown as in FIG. 1. (E) A model depicts PKD1-mediated threonine (Thr) phosphorylation (\textcircled{P} that differentiates the major role of HIF-2α from that of HIF-1α in the hypoxic response. Whereas nonphosphorylated competes with Myc for Sp1 binding, resulting in NBS1 and MSH2 repression and consequently DNA damage, phosphorylated HIF-2α primarily engages in the canonical hypoxia-responsive pathway.

FIG. 8 shows HIF-1α mediates specific inhibition of NBS1 by hypoxia. HCT116 and HCT116 TP53^{-/-} cells transfected with siRNA targeting HIF1A or EPAS1 were maintained in normoxia (N) or subjected to hypoxic treatment (1% O₂, 16 h). Mock transfection (−) and luciferase siRNA (luc) were used as negative controls. NBS1, MRE11A, and RAD50 mRNA levels were determined with quantitative real-time PCR. Representative results from three independent experiments in triplicate are presented as means±standard errors.

FIG. 9 shows hypoxic inhibition of NBS1 expression in HCT116 cells. HCT116 cells were maintained in normoxia or subjected to hypoxic treatment for 8 (H8) or 16 (H16) h. A series of Western blot analysis were performed to detect indicated protein levels.

FIG. 10 shows hypoxic stress induces DNA double-strand breaks. U-2 OS cells were maintained under normoxia (N), or subjected to hypoxic (H) or desferrioxamine (D) conditions for 72 h and stained with antibodies against γ-H2AX (red) and 53BP1 (green). Representative fields are presented together with merged images (yellow). In contrast to normoxic cells, hypoxic cells exhibited a marked increase in γ-H2AX foci. Some of the hypoxic cells as well as those treated with desferrioxamine manifested intensified staining.

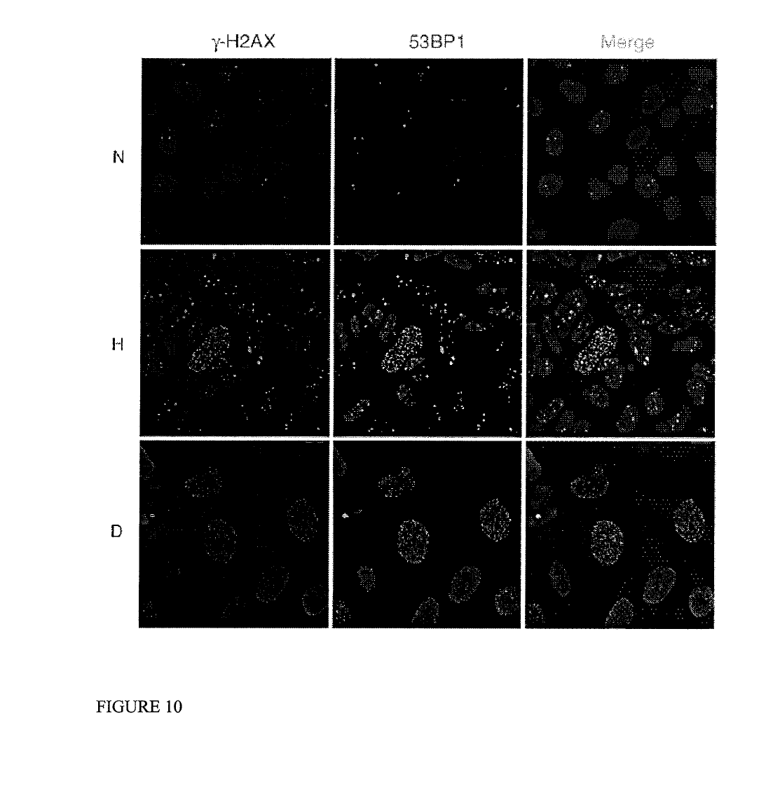

FIG. 11 shows forced expression of HIF-la induces DNA double-strand breaks. U-2 OS cells were infected with adenoviruses expressing HIF-1α variants as in FIG. 2. γ-H2AX and 53BP1 foci were determined by immunofluorescence. Additional fields are presented with merged images only. Again, with the exception of Ad-HIF-1α(1-167), all of the HIF-1α variants induce DNA DSBs, as indicated by the significant increase in the number of co-localized γ-H2AX and 53BP1 foci.

FIG. 12 shows specific NBS1 repression by non-phosphorylated PAS-B in U-2 OS cells. U-2 OS cells were transfected with PAS1B, PAS2B and their mutants, as indicated. NBS1, MRE11A, and RAD50 mRNA levels were determined with quantitative real-time PCR and presented as in FIG. 1.

FIG. 13 shows specific NBS1 repression by non-phosphorylated PAS-B is p53-independent PAS1B, PAS2B and their relevant mutants were transfected into HCT116 and HCT116 TP53^{-/-} cells. NBS1, MRE11A, and RAD50 mRNA levels were determined with quantitative real-time PCR and presented as in FIG. 1.

FIG. 14 shows HIF-1α PAS-B expression promotes malignant properties in HCT116 and U-2 OS cells. A, retrovirally infected HCT116 cells expressing EYFP, HIF-1α PAS-B, and mutant were assayed for Matrigel invasion. Images of cells on the membrane side were taken 24 h later. B, U-2 OS cells infected as above were assayed for anchorage-independent growth on soft agar. HIF-1α PAS-B expressed cells gave rise to formation of colonies 40 times more than others. HCT116 cells (known to grow on soft agar) were used as a positive control. Mock, uninfected cells.

FIG. 15 shows HIF-1α PAS-B expression in HCT116 and U-2 OS cells results in striking phenotypic changes. A, Cells as indicated were infected by retroviruses expressing EYFP, EYFP-PAS-B fusion, and EYFP-PAS-B mutant. Both HIF-1α PAS-B expressed HCT116 and U-2 OS cells exhibit fibroblast-like morphology. B, U-2 OS and those infected with retroviruses as above were analyzed by immunoblotting individual protein expression as indicated. There is a marked decrease in the epithelial marker β-catenin in cells expressing HIE-1α PAS-B.

FIG. 16 shows HIF-1α PAS-B expression accelerates tumor formation in a xenografted mouse model. Female BALB/c-nu/nu mice were subjected to bilateral, subcutaneous injections in the back with 1 million of U-2 OS cells, or those expressing EYFP, HIF-1α PAS-B, or the HIF-1α PAS-B mutant. A total of ten mice were divided into two groups with one type of cells injected on one side and another on the other side. Three weeks after, only those injected with HIF-1α PAS-B expressed cells developed tumor nodules (as circled).

FIG. 17 shows hypoxia inhibits CDC25A expression independent of the ATR-Chk1 pathway. A, HCT116 cells were cultured under normoxic (N) of hypoxic (H; 1% O₂) conditions for 16 h. CDC25A and PGK1 mRNA levels were determined by real-time RT-PCR. The experiments were repeated three times in triplicate, and representative results were presented in mean±standard error. B, CDC25A and HIF-1α protein levels were analyzed by Western blot analysis. Cells irradiated with 50 J/m² of UV-C light for 2 h were included as a control for CDC25A proteolysis. β-actin levels served as a loading control. C, Chk1 expression in HCT116 cells was knocked down with siRNA transfection for 24 h. A luciferase gene siRNA (Luc) was used as a negative control. Western blot analysis was performed to determine Chk1 protein levels. D, HCT116 cells transfected with CHK1 siRNA were analyzed for hypoxic effects on CDC25A expression with real-time RT-PCR, as above.

FIG. 18 shows hypoxia inhibits CDC25A expression irrespective of the p53 status. Wild-type HCT116 (WT) and those deficient in p53 (p53^{-/-}) or p21^{cip1} (p21^{-/-}), as well as MCF7, Hep3B and HeLa cells were examined for hypoxic effects on CDC25A expression by real-time RT-PCR. PGK1 mRNA levels were also determined as a control for the induction of hypoxia-responsive genes.

FIG. 19 shows HIF-1α, but not HIF-2α, is required for hypoxic repression of CDC25A gene. A, HCT116 cells were transfected for 24 h with siRNAs targeting HIF1A, EPAS1, and luciferase gene (Luc) respectively, and subsequently cultured under normoxic (N) or hypoxic (H) conditions for 16 h. Mock transfected cells (−) served as a negative control. CDC25A and PGK1 mRNA levels were determined by real-time RT-PCR, as above. B, The siRNA-transfected cells were assayed for HIF-1α and HIF-2α protein levels by Western blotting. β-actin levels served as a loading control.

FIG. 20 shows hypoxia inhibits CDC25A transcription by targeting Myc-binding activity in the promoter that lacks a canonical E-box. A, HCT116 cells were transfected with the 0.7 wtNP-GL3¹⁴ CDC25A reporter (containing the natural promoter and the Myc-binding region 3) in the absence (−Myc) or the presence (+Myc) of a Myc expression vector. After hypoxic treatment for 20 h, reporter activities as a function of relative luciferase activity (RLU) were determined as described previously. Results represent means±standard deviations (n=4). B, The wild-type CDC25A reporter 0.7 wtNP-GL3 (Wt) and one with mutated Myc binding region 3 (Mut) were used to test for the requirement of the Myc-binding site for hypoxic repression. C, A CDC25A promoter only reporter, CDC25A-luc, was transfected into HCT116 cells in the presence of an increasing amount (0, 100, 200 ng) of the Myc expression vector. Cells were maintained either in normoxia or subjected to hypoxia. D, The activities of CDC25A-luc were examined in the presence of MYC siRNA under normoxia and hypoxia. Reporter activities were determined as above. E, Individual knockdown of HIF1A, MYC, and SP1 gene expression was carried out with respective siRNAs in HCT116 cells. HIF-1α, Myc, Sp1, and β-actin protein levels under normoxia or after a 16-h hypoxic treatment were determined by Western blot with the corresponding antibodies as indicated.

FIG. 21 shows selective Myc displacement in CDC25A gene by hypoxia. A, A schematic representation of portion of CDC25A gene (locus AF527417). Exon 1, 2, and 3 (Ex 1, Ex 2, and Ex 3) were depicted in shaded boxes, and the Myc-binding region 3 (MB3) in a black box. PCR primers span the natural promoter (NP) and MB3 were indicated in reference to the transcription start site. B, HCT116 cells were cultured under normoxic (N) or hypoxic (H) conditions for 16 h. Chromatin immunoprecipitations were performed with antibodies against RNA polymerase II (Pol II), Sp1, HIF-1α, Myc, p53 and IgG. PCR amplified DNA fragments represent binding of the transcription factors to the CDC25A NP and MB3 regions, respectively.

FIG. 22 shows HIF-1α is insufficient for CDC25A repression. A, HCT116 cells were infected with recombinant adenoviruses expressing various HIF-1α mutants [Ad-HIF1α ΔODD, Ad-HIF1α ΔODD(LCLL), and Ad-HIF1α(1-329)] or HIF-2α (Ad-HIF2α) overnight. CDC25A and PGK1 mRNA levels were determined by real-time RT-PCR, as previously. A GFP recombinant adenovirus served as a negative control. B, HCT116 cells were transfected with a CDC25A promoter only reporter (top panel) or an HIΦ-1α(HIF-1α PP) as specified. Desferrioxamine (D, 100 μM) was used for normoxic stabilization of endogenous HIF-1α. Reporter activities were determined as above. C, HCT116 cells were infected with Ad-HIF1α ΔODD (+) or Ad-GFP (−) for 24 h. Chromatin immunoprecipitations were performed with indicated antibodies. The CDC25A NP and MB3 regions and the CDKN1A proximal promoter were PCR amplified respectively with the same batch of precipitated lysates.

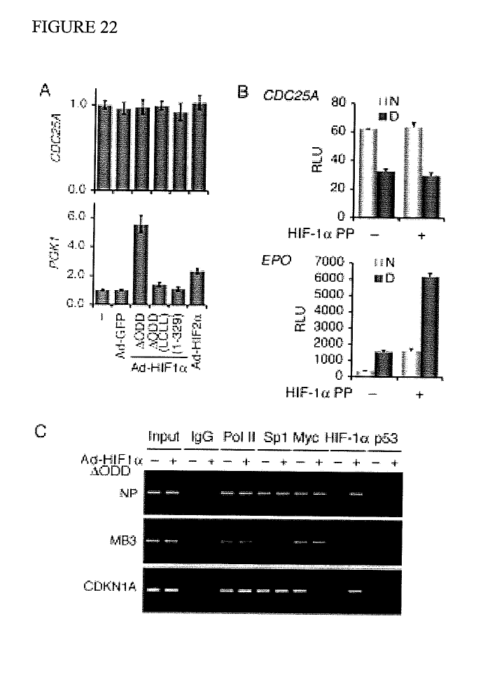

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10″as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

As used herein, the terms “manage,” “managing” and “management” refer to the beneficial effects that a subject derives from administration of a prophylactic or therapeutic agent, which does not result in a cure of the disease or diseases. In certain embodiments, a subject is administered one or more prophylactic or therapeutic agents to “manage” a disease so as to prevent the progression or worsening of the disease or diseases.

As used herein, the terms “prevent”, “preventing” and “prevention” refer to the methods to avert or avoid a disease or disorder or delay the recurrence or onset of one or more symptoms of a disorder in a subject resulting from the administration of a prophylactic agent.

As used herein, “treat” or “treatment” means a postponement of development of the symptoms associated with uncontrolled metastatic tumor growth and/or a reduction in the severity of such symptoms that will or are expected to develop. The terms further include ameliorating existing uncontrolled or unwanted metastatic tumor growth-related symptoms, preventing additional symptoms, and ameliorating or preventing the underlying metabolic causes of symptoms. Thus, the terms denote that a beneficial result has been conferred on a mammalian subject with a metastasis-associated disease or symptom, or with the potential to develop such metastatic disease or symptom. In particular, the term encompasses administration of a composition that prevents metastatic tumor formation and/or inhibits or kills existing metastatic tumors, with resulting clinical benefit. Thus the term “metastatic tumor growth” means the establishment and growth of metastatic tumors, i.e., tumors that have spread from the site of a primary tumor.

The term “metastasis” means the ability of tumor cells to invade host tissues and metastasize to distant, often specific organ sites. As is known, this is the salient feature of lethal tumor growths. Metastasis formation occurs via a complex series of unique interactions between tumor cells and normal host tissues and cells. In the context of the present invention, HIF-1α activity is critical in the metastatic growth of tumors, i.e., the growth of metastases, particularly under hypoxic conditions. As hypoxic tumors are also the most aggressive and resistant to traditional chemotherapy, agents modulating HIF-1α expression and/or function provide a novel therapy against metastatic tumors, particularly chemo-resistant tumors. “Metastasis” is distinguished from invasion. As described in “Understanding Cancer Series: Cancer,” http://www.cancer.gov/cancertopics/understandingcancer/cancer, invasion refers to the direct migration and penetration by cancer cells into neighboring tissues.

The term “pharmaceutically acceptable carrier” is intended to include formulation used to stabilize, solubilize and otherwise be mixed with active ingredients to be administered to living animals, including humans. This includes any and all solvents, dispersion media, coatings, antibacterial and antifungal agents, isotonic and absorption delaying agents, and the like, compatible with pharmaceutical administration. The use of such media and agents for pharmaceutically active substances is well known in the art. See e.g., Remington: The Science and Practice of Pharmacy 20th Ed. (Lippincott, Williams & Wilkins 2003). Except insofar as any conventional media or agent is incompatible with the active compound, such use in the compositions is contemplated.

As used herein, the terms “small interfering RNA” (“siRNA”) or “short interfering RNAs”) refer to an RNA (or RNA analog) comprising between about 10-50 nucleotides (or nucleotide analogs) which is capable of directing or mediating RNA interference. As used herein, “shRNA” should be distinguished from siRNA. As described in Harmon et al., “Unlocking the potential of the human genome with RNA interference,” Nature 431, 371-378 (16 Sep. 2004), shRNA involves expressing mimics of miRNAs in the form of short hairpin RNAs (shRNAs) from RNA polymerase II or III promoters. shRNAs typically have stems ranging from 19 to 29 nucleotides in length, and with various degrees of structural similarity to natural miRNAs. Because these triggers are encoded by DNA vectors, they can be delivered to cells in any of the innumerable ways that have been devised for delivery of DNA constructs that allow ectopic mRNA expression. These include standard transient transfection, stable transfection and delivery using viruses ranging from retroviruses to adenoviruses. Each shRNA expression construct gives rise to a single siRNA.

As used herein, the term “in combination” refers to the use of more than one prophylactic and/or therapeutic agents. The use of the term “in combination” does not restrict the order in which prophylactic and/or therapeutic agents are administered to a subject with a disorder, e.g., hyperproliferative cell disorder, especially cancer. A first prophylactic or therapeutic agent can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject which had, has, or is susceptible to a disorder. The prophylactic or therapeutic agents are administered to a subject in a sequence and within a time interval such that the agent of the invention can act together with the other agent to provide an increased benefit than if they were administered otherwise. Any additional prophylactic or therapeutic agent can be administered in any order with the other additional prophylactic or therapeutic agents.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. General

Hypoxia (a state in which oxygen demand exceeds supply) is a powerful modulator of gene expression. The physiologic response to hypoxia involves enhanced erythropoiesis (Jelkman, Physiol. Rev. 72:449-489 (1992)), neovascularization in ischemic tissues (White et al., Circ. Res. 71:1490-1500 (1992)) and a switch to glycolysis-based metabolism (Wolfe et al., Eur. J. Biochem. 135:405-412 (1983)). These adaptive responses either increase oxygen delivery or activate alternate metabolic pathways that do not require oxygen. The gene products involved in these processes, include, for example: (i) EPO, encoding erythropoietin, the primary regulator of erythropoiesis and thus a major determinant of blood oxygen-carrying capacity (Jiang et al., J. Biol. Chem. 271(30):17771-78 (1996); (ii) VEGF, encoding vascular endothelial growth factor, the primary regulator of angiogenesis and thus a major determinant of tissue perfusion (Levy et al., J. Biol. Chem. 270:13333 (1995); Liu et al., Circ. Res. 77:638 (1995); Forsythe et al., Mol. Cell. Biol. 16:4604 (1996)); (iii) ALDA, ENO1, LDHA, PFKL, and PGK1, encoding the glycolytic enzymes aldolase A, enolase 1, lactate dehydrogenase A, phosphofructokinase L, and phosphoglycerate kinase 1, respectively, which provide a metabolic pathway for ATP generation in the absence of oxygen (Firth et al., Proc. Natl. Acad. Sci., USA 91:6496 (1994); Firth et al., J. Biol. Chem. 270:21021 (1995); Semenza et al., J. Biol. Chem. 269:23757 (1994)); (iv) H01 and iNOS, encoding heme oxygenase 1 and inducible nitric oxide synthase, which are responsible for the synthesis of the vasoactive molecules carbon monoxide and nitric oxide, respectively (Lee et al., J. Biol. Chem. 272:5375; Melillo et al. J. Exp. Med. 182:1683 (1995)).

An important mediator of these responses is the interaction of a transcriptional complex comprising a DNA binding, hypoxia inducible factor protein, with its cognate

DNA recognition site, a hypoxia-responsive element (HRE) located within the promoter/enhancer elements of hypoxia-inducible genes. HREs consist of an hypoxia inducible factor protein binding site (that contains the core sequence 5′-CGTG-3′) as well as additional DNA sequences that are required for function, which in some elements includes a second binding site.

HIF-1 is a heterodimeric protein composed of two subunits: (i) a constitutively expressed beta β) subunit (shared by other related transcription factors) and (ii) an alpha (α) subunit (see, e.g., WO 96/39426, International Application No. PCT/US96/10251 describing the recent affinity purification and molecular cloning of HIF-1α) whose accumulation is regulated by a post-translational mechanism such that high levels of the alpha subunit can only be detected during hypoxic conditions. Both subunits are members of the basic helix-loop-helix (bHLH)-PAS family of transcription factors. These domains regulate DNA binding and dimerization.

Whereas, HIF-1β (ARNT) is expressed constitutively at a high level, accumulation of HIF-1α in the cell is sensitive to oxygen concentration such that high levels are detected only during hypoxia. This observation has led to a proposed mechanism for target gene activation whereby oxygen concentration is detected by a sensor protein and through a complex signaling mechanism leads to stabilization of the HIF-1α subunit. HIF-1α is then available to complex with HIF-1α and bind selectively to HRE sites in the promoter/enhancer of the target gene(s).

The transcription factor HIF-1α serves as a master regulator of oxygen homeostasis by activating expression of various hypoxia-responsive genes, such as those for angiogenesis and glycolysis (Wenger, 2002; Giaccia et al, 2003; Huang and Bunn, 2003; Pugh and Ratcliffe, 2003; Semenza, 2003; Poellinger and Johnson, 2004; Kaelin, 2005). Interestingly, HIF-2α, a close member of the HIF-α family, seems to exert different biological functions in vivo (Tian et al, 1998; Compernolle et al, 2002; Scortegagna et al, 2003). Apart from binding to the hypoxia-responsive element for gene activation, HIF-1α also functions via the HIF-1α-Myc pathway, by which HIF-1α competes with the transcription activator Myc for Sp1 binding in the target gene promoter, resulting in transcriptional downregulation of the DNA mismatch repair genes (Koshiji et al, 2005). To explore the differential role of HIF-1α and HIF-2α in DNA repair, the molecular basis that accounts for their distinct functions in genetic instability was investigated. The data demonstrated that the phosphorylation status of a highly conserved threonine in the PAS-B domain distinguishes HIF-1α from HIF-2α in DNA repair gene expression. HIF-1α can be found, for example, in Genbank as accession no. U22431. The PAS-B domain comprises codons 194-329. The amino acid sequence for HIF-1α is:

(SEQ ID NO: 1)

MEGAGGANDKKKISSERRKEKSRDAARSRRSKESEVFYELAHQLPLPHNV
SSHLDKASVMRLTISYLRVRKLLDAGDLDIEDDMKAQMNCFYLKALDGFV
MVLTDDGDMIYISDNVNKYMGLTQFELTGHSVFDFTHPCDHEEMREMLTH
RNGLVKKGKEQNTQRSFFLRMKCTLTSRGRTMNIKSATWKVLHCTGHIHV
YDTNSNQPQCGYKKPPMTCLVLICEPIPHPSNIEIPLDSKTFLSRHSLDM
KFSYCDERITELMGYEPEELLGRSIYEYYHALDSDHLTKTHHDMFTKGQV
TTGQYRMLAKRGGYVWVETQATVIYNTKNSQPQCIVCVNYVVSGIIQHDL
IFSLQQTECVLKPVESSDMKMTQLFTKVESEDTSSLFDKLKKEPDALTLL
APAAGDTIISLDFGSNDTETDDQQLEEVPLYNDVMLPSPNEKLQNINLAM
SPLPTAETPKPLRSSADPALNQEVALKLEPNPESLELSFTMPQIQDQTPS
PSDGSTRQSSPEPNSPSEYCFYVDSDMVNEFKLELVEKLFAEDTEAKNPF
STQDTDLDLEMLAPYIPMDDDFQLRSFDQLSPLESSSASPESASPQSTVT
VFQQTQIQEPTANATTTTATTDELKTVTKDRMEDIKILIASPSPTHIHKE
TTSATSSPYRDTQSRTASPNRAGKGVIEQTEKSHPRSPNVLSVALSQRTT
VPEEELNPKILALQNAQRKRKMEHDGSLFQAVGIGTLLQQPDDHAATTSL
SWKRVKGCKSSEQNGMEQKTIILIPSDLACRLLGQSMDESGLPQLTSYDC
EVNAPIQGSRNLLQGEELLRALDQVN

The nucleic acid sequence of HIF-1α is:

(SEQ ID NO: 2)

tactagtgcc acatcatcac catatagaga tactcaaagt
cggacagcct caccaaacag agcaggaaaa ggagtcatag
aacagacaga aaaatctcat ccaagaagcc ctaacgtgtt
atctgtcgct ttgagtcaaa gaactacagt tcctgaggaa
gaactaaatc caaagatact agctttgcag aatgctcaga
gaaagcgaaa aatggaacat gatggttcac tttttcaagc
agtaggaatt ggaacattat tacagcagcc agacgatcat
gcagctacta catcactttc ttggaaacgt gtaaaaggat
gcaaatctag tgaacagaat ggaatggagc aaaagacaat
tattttaata ccctctgatt tagcatgtag actgctgggg
caatcaatgg atgaaagtgg attaccacag ctgaccagtt
atgattgtga agttaatgct cctatacaag gcagcagaaa
cctactgcag ggtgaagaat tactcagagc tttggatcaa
gttaactgag ctttttctta atttcattcc tttttttgga
cactggtggc tcactaccta aagcagtcta tttatatttt
ctacatctaa ttttagaagc ctggctacaa tactgcacaa
acttggttag ttcaattttt gatccccttt ctacttaatt
tacattaatg ctctttttta gtatgttctt taatgctgga
tcacagacag ctcattttct cagttttttg gtatttaaac
cattgcattg cagtagcatc attttaaaaa atgcaccttt
ttatttattt atttttggct agggagttta tccctttttc
gaattatttt taagaagatg ccaatataat ttttgtaaga
aggcagtaac ctttcatcat gatcataggc agttgaaaaa
tttttacacc ttttttttca cattttacat aaataataat
gctttgccag cagtacgtgg tagccacaat tgcacaatat
attttcttaa aaaataccag cagttactca tggaatatat
tctgcgttta taaaactagt ttttaagaag aaattttttt
tggcctatga aattgttaaa cctggaacat gacattgtta
atcatataat aatgattctt aaatgctgta tggtttatta
tttaaatggg taaagccatt tacataatat agaaagatat
gcatatatct agaaggtatg tggcatttat ttggataaaa
ttctcaattc agagaaatca tctgatgttt ctatagtcac
tttgccagct caaaagaaaa caatacccta tgtagttgtg
gaagtttatg ctaatattgt gtaactgata ttaaacctaa
atgttctgcc taccctgttg gtataaagat attttgagca
gactgtaaac aagaaaaaaa aaatcatgca ttcttagcaa
aattgcctag tatgttaatt tgctcaaaat acaatgtttg
attttatgca ctttgtcgct attaacatcc tttttttcat
gtagatttca ataattgagt aattttagaa gcattatttt
aggaatatat agttgtcaca gtaaatatct tgttttttct
atgtacattg tacaaatttt tcattccttt tgctctttgt
ggttggatct aacactaact gtattgtttt gttacatcaa
ataaacatct tctgtgga

Disclosed is a polypeptide comprising the PAS-B of HIF-1α, including mutations in the PAS-B domain. Such mutations can comprise 1,2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 50, 51, 52, 53, 54, 55, 56, 57, 58, 59, 60, or more amino acid mutations that differ from the HIF-1α protein sequence given above. Also disclosed are mutations to the PAS-B domain in the nucleic acid. Such mutations can comprise 1,2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 50, 51, 52, 53, 54, 55, 56, 57, 58, 59, 60, or more nucleic acid mutations that differ from the HIF-1α nucleic acid sequence given above.

The adaptive response to hypoxia is likely to have been highly conserved throughout evolution. Accordingly, hypoxia inducible factor proteins can occur in a wide variety of species including non-mammalian vertebrates and non-vertebrates such as insects. See, for example, Bacon et al., Biochem. Biophys. Res. Comm., 249:811-816 (1998), which reports the functional similarity between the Sima basic-helix-loop-helix PAS protein from Drosophila and the mammalian HIF-1α protein.

Nucleic acid and amino acid sequences for non-mammalian hypoxia inducible factor proteins may be obtained by the skilled artisan by a variety of techniques, for example by cross-hybridization or amplification using all or a portion of the sequences referred to herein. Once the sequence encoding a candidate hypoxia inducible factor protein has been determined, the localization of portions of the protein sufficient to bind to HREs and dimerize with HIF-1β may be determined using, e.g., the same types of techniques used to determine the location of those domains within the human HIF-1α protein. Relevant domains of non-mammalian hypoxia inducible factor proteins useful in the compositions and methods of this invention may also be produced synthetically or by site-directed manipulations of the DNA encoding known mammalian hypoxia inducible factor proteins. It is also expected that the sequence motifs in common among various mammalian and non-mammalian hypoxia inducible factor proteins will suggest consensus sequences that, while perhaps not occurring naturally in any species, would nevertheless produce domains useful in the methods and compositions of this invention. All that is required in order to substitute such non-mammalian hypoxia inducible factor protein domains for the human HIF-1α protein domains exemplified herein is that they be able to bind to HREs and dimerize with HIF-1β.

Of particular interest is the ability of hypoxia inducible factor proteins, for example, HIF-1α and its associated PAS-B domain, to induce expression of hypoxia-inducible genes such as, for example VEGF and the like, resulting in the amelioration of symptoms through promotion of collateral blood vessel growth. For example, although the HIF-1α subunit is unstable during normoxic conditions, overexpression of this subunit in cultured cells under normal oxygen levels is capable of inducing expression of genes normally induced by hypoxia. This shows that a useful gene therapy strategy might be to express high levels of the HIF-1α PAS-B subunit in ischemic heart in vivo using a recombinant plasmid or viral vector. An alternative strategy is to modify the HIF-1α subunit such that it no longer is destabilized by normoxic conditions and would therefore be more potent, particularly when the subject being treated is not actually ischemic.

“Hypoxia” means the state in which oxygen demand exceeds supply. “Hypoxia-inducible genes” means genes containing one or more hypoxia responsive elements (HREs; binding sites) within sequences mediating transcriptional activation in hypoxic cells.

Hypoxia inducible factor means a DNA binding protein/transcription factor the expression of which is upregulated under hypoxic conditions, that recognizes and binds to a hypoxia responsive element core sequence within a gene and thereby activates such gene. Hypoxia-associated disorders include, for example, ischemic heart disease, peripheral vascular disease, ischemic disease of the limb, and the like.

### C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### 1. Homology/Identity

It is understood that one way to define any known variants and derivatives or those that might arise, of the disclosed genes and proteins herein is through defining the variants and derivatives in terms of homology to specific known sequences. Specifically disclosed are variants of HIF-1α and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Furthermore, it can be desirable to mutate the PAS-B domain of HIF-1α, as disclosed herein, such mutations can be used in the treatment of disease, including ischemia. One of skill in the art could ascertain these mutations and which would be effective. Examples of such mutations are given in Example 1.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 2. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987: 154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### 3. Nucleic Acids

There are a variety of molecules disclosed herein that are nucleic acid based, including for example the nucleic acids that encode, for example, HIF-1α as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

The term “nucleic acids” (also referred to as polynucleotides) encompasses RNA as well as single and double-stranded DNA, cDNA and oligonucleotides.

Nucleic acids also encompass isolated nucleic acid sequences, including sense and antisense oligonucleotide sequences, e.g., derived from the HIF-1α PAS-B sequences. HIF-1α -derived sequences may also be associated with heterologous sequences, including promoters, enhancers, response elements, signal sequences, polyadenylation sequences, and the like. As used herein, the phrase “isolated” means a polynucleotide that is in a four that does not occur in nature. One means of isolating polynucleotides is to probe a human tissue-specific library with a natural or artificially designed DNA probe using methods well known in the art. DNA probes derived from the human HIF-1α gene, or the PAS-B domain of HIF-1α are particularly useful for this purpose. DNA and cDNA molecules that encode invention polypeptides can be used to obtain complementary genomic DNA, cDNA or RNA from human, mammalian, or other animal sources, or to isolate related cDNA or genomic clones by the screening of cDNA or genomic libraries, by methods described in more detail below.

Furthermore, the nucleic acids can be modified to alter stability, solubility, binding affinity, and specificity. For example, invention-derived sequences can further include nuclease-resistant phosphorothioate, phosphoroamidate, and methylphosphonate derivatives, as well as “protein nucleic acid” (PNA) formed by conjugating bases to an amino acid backbone as described in Nielsen et al., Science, 254:1497, (1991). The nucleic acid may be derivatized by linkage of the α-anomer nucleotide, or by formation of a methyl or ethyl phosphotriester or an alkyl phosphoramidate linkage. Furthermore, the nucleic acid sequences of the present invention may also be modified with a label capable of providing a detectable signal, either directly or indirectly. Exemplary labels include radioisotopes, fluorescent molecules, biotin, and the like.

Also disclosed are nucleic acids which differ from the nucleic acids encoding a human HIF-1α, but which have the same phenotype, i.e., encode substantially the same amino acid sequence, respectively. Phenotypically similar nucleic acids are also referred to as “functionally equivalent nucleic acids”. As used herein, the phrase “functionally equivalent nucleic acids” encompasses nucleic acids characterized by slight and non-consequential sequence variations that will function in substantially the same manner to produce the same or substantially the same protein product(s) as the nucleic acids disclosed herein. In particular, functionally equivalent nucleic acids encode proteins that are the same as those disclosed herein or that have conservative amino acid variations. For example, conservative variations include substitution of a non-polar residue with another non-polar residue, or substitution of a charged residue with a similarly charged residue. These variations include those recognized by skilled artisans as those that do not substantially alter the tertiary structure of the protein.

A structural gene is that portion of a gene comprising a DNA segment encoding a protein, polypeptide or a portion thereof, and excluding the 5′ sequence which drives the initiation of transcription. The structural gene may be one which is normally found in the cell or one which is not normally found in the cellular location wherein it is introduced, in which case it is termed a heterologous gene. A heterologous gene may be derived in whole or in part from any source know to the art, including a bacterial genome or episome, eukaryotic, nuclear or plasmid DNA, cDNA, vital DNA or chemically synthesized DNA. A structural gene may contain one or more modifications in either the coding or the untranslated regions which could affect the biological activity or the chemical structure of the expression product, the rate of expression or the manner of expression control. Such modifications include, but are not limited to, mutations, insertions, deletions and substitutions of one or more nucleotides. The structural gene may constitute an uninterrupted coding sequence or it may include one or more introns, bound by the appropriate splice junctions. The structural gene may be a composite of segments derived from a plurality of sources, naturally occurring or synthetic. The structural gene can also encode a fusion protein. It is contemplated that the introduction of recombinant DNA molecules containing the structural gene/transactivator complex includes constructions wherein the structural gene and the transactivator are each derived from different sources or species.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to, for example, HIF-1α as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

c) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

### 4. Inhibitors of HIF-1α

Disclosed herein are inhibitors of HIF-1α. Also disclosed is the inhibition of any of the molecules in the hypoxia pathway, such as the Myc pathway, including Sp1 or HIF. Any suitable source of HIF-1α may be employed as an inhibitor target in the present method. The enzyme can be derived, isolated, or recombinantly produced from any source known in the art, including yeast, microbial, and mammalian, that will permit the generation of a suitable product that can generate a detectable reagent or will be biologically active in a suitable assay. In one embodiment, the HIF-1α is of human, bovine, or other mammalian origin. See, e.g., Williams, et al., Anal. Biochem. 113:336 (1985); Kirschmann et al., supra; Cancer Res. 62:4478-83 (2002). A functional fragment or a derivative of HIF-1α, such as PAS-B, that still substantially retains its enzymatic activity can also be used.

Hypoxic conditions can be induced or naturally occurring. Hypoxic areas frequently occur in the interior of solid tumor. Hypoxia can also be induced in vivo, particularly in experimental animal models, using diminution or cessation of arterial blood flow to tumor or the administration of vasoconstrictive compounds. See, e.g., U.S. Pat. No. 5,646,185. Exemplary vasoconstrictive compounds include adrenergic direct and indirect agonists such as norepinephrine, epinephrine, phenylephrine, and cocaine. The presence of a hypoxic region in a solid tumor present in a subject can be observed by a number of methods currently known in the art, including nuclear magnetic resonance (NMR) and oxygen electrode pO₂ histography. Such methods may be used in the context of the present invention, to identify hypoxic treatment target regions and to guide in administering treatment compositions to such regions. In vitro, hypoxic conditions can be induced using any suitable method. For example, cells can be maintained under anoxic (<0.1% O₂) conditions at 37° C. within an anaerobic chamber or under hypoxic (1 to 2% O₂) conditions at 37° C. within a modular incubator chamber filled with 5% CO₂ and 1 to 2% O₂ balanced with N₂. See, e.g., Erler et al., Mol. Cell. Biol. 24:2875-89 (2004).

A compound is an inhibitor of HIF-1α expression or biological activity when the compound reduces the expression or activity or HIF-1α relative to that observed in the absence of the compound. In one embodiment, a compound is an inhibitor of HIF-1α when the compound reduces the incidence of metastasis relative to the observed in the absence of the compound and, in further testing, inhibits metastatic tumor growth. The tumor inhibition can be quantified using any convenient method of measurement. The incidence of metastasis can be assessed by examining relative dissemination (e.g., number of organ systems involved) and relative tumor burden in these sites. Metastatic growth can be ascertained by microscopic or macroscopic analysis, as appropriate. Tumor metastasis can be reduced by about 10%, 20%, 30%, 40%, 50%, 60%, 70%, 80%, 90%, 95% or greater. In some embodiments, the compound can be assessed relative to other compounds that do not impact HIF-1α expression or biological activity. The test compounds can be administered at the time of tumor inoculation, after the establishment of primary tumor growth, or after the establishment of local and/or distant metastases. Single or multiple administration of the test compound can be given using any convenient mode of administration, including but not limited to intravenous, intraperitoneal, intratumoral, subcutaneous, and intradermal.

Any suitable cell expressing HIF-1α may be employed with the present methods. As used herein, the term “cell” includes a biological cell. The cell can be human or nonhuman. The cell can be freshly isolated (i.e., primary) or derived from a short term- or long term-established cell line. Exemplary biological cell lines include MDA-MB 231 human breast cancer cells, MDA-MB 435 human breast cancer cells, U-87 MG glioma, SCL1 squamous cell carcinoma cells, CEM, HeLa epithelial carcinoma, and Chinese hamster ovary (CHO) cells. Such cell lines are described, for example, in the Cell Line Catalog of the American Type Culture Collection (ATCC, Rockville, Md.).

A cell can express the HIF-1α or its promoter endogenously or exogenously (e.g., as a result of the stable transfer of genes). Endogenous expression by a cell as provided herein can result from constitutive or induced expression of endogenous genes.

Exogenous expression by a cell as provided herein can result from the introduction of the nucleic acid sequences encoding HIF-1α or a biologically active fragment thereof, or a HIF-1α promoter nucleic acid sequence. Transformation may be achieved using viral vectors, calcium phosphate, DEAE-dextran, electroporation, cationic lipid reagents, or any other convenient technique known in the art. The manner of transformation useful in the present invention is conventional and is exemplified in Current Protocols in Molecular Biology (Ausubel, et al., eds. 2000). Exogenous expression of HIF-1α or its promoter can be transient, stable, or some combination thereof. Exogenous expression of the enzyme can be achieved using constitutive promoters, e.g., SV40, CMV, and the like, and inducible promoters known in the art. Suitable promoters are those that will function in the cell of interest.

The HIF-1α -expressing cell can be contacted with the compound in any suitable manner for any suitable length of time. For tumor regions that are accessible to hypodermic delivery of agent, it may be desirable to inject the inhibitory compounds directly into the hypoxic region. The cells can be contacted with the compound more than once during incubation or treatment. Typically, the dose required for an antibody is in the range of about 1 μg/ml to 1000 μg/ml, more typically in the range of 100 μg/ml to 800 μg/ml. The exact dose can be readily determined from in vitro cultures of the cells and exposure of the cell to varying dosages of the compound. Typically, the length of time the cell is contacted with the compound is about 5 minutes, about 15 minutes, about 30 minutes, about 1 hour, about 4 hours, about 12 hours, about 36 hours, about 48 hours to about 3 days, more typically for about 24 hours. For in vitro invasion assays, any suitable matrix may be used. In one embodiment, the matrix is reconstituted basement membrane Matrigel™ matrix (BD Sciences).

Inhibitor compounds are those molecules that inhibit or reduce HIF-1α function activity, preferably to reduce metastatic tumor growth. Such inhibition can occur through direct binding of one or more critical binding residues of HIF-1α or through indirect interference including steric hindrance, enzymatic alteration of the HIF-1α, inhibition of transcription or translation, destabilization of mRNA transcripts, impaired export, processing, or localization of HIF-1α, and the like. As used herein, the term “inhibitor compound” includes both protein and non-protein moieties. In some embodiments, the inhibitors are small molecules. Preferably, the inhibitors are compounds with sufficient specificity to avoid systemic toxicity to collagen-rich tissues.

A variety of different test inhibitory compounds may be identified using the method as provided herein. Test inhibitory compounds can encompass numerous chemical classes. In certain embodiments, they are organic molecules, preferably small organic compounds having a molecular weight of more than 50 and less than about 2,500 daltons. Test inhibitory compounds can comprise functional groups necessary for structural interaction with proteins, particularly hydrogen bonding, and may include at least an amine, carbonyl, hydroxyl or carboxyl group, preferably at least two of the functional chemical groups. The test inhibitory compounds can comprise cyclical carbon or heterocyclic structures and/or aromatic or polyaromatic structures substituted with one or more of the above functional groups. Test inhibitory compounds also include biomolecules like peptides, saccharides, fatty acids, steroids, purines, pyrimidines, derivatives, structural analogs or combinations thereof. Test inhibitory compounds of interest also can include peptide and protein agents, such as antibodies or binding fragments or mimetics thereof, e.g., Fv, F(ab′)₂ and Fab, as described further below.

Test inhibitory compounds also can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The inhibitor can also be prepared and administered as prodrugs. As is known, a pro-drug is a derivative of an active drug, often a relatively simple derivative, whose properties are considerably reduced, compared to those of the drug. The pro-drug is converted to the active drug in the region of the intended action, in this case a tumor or site of metastasis.

In the present composition, a HIF-1α inhibitory compound can be synthesized as a pro-drug, which is converted by hypoxic conditions to the active inhibitor. Other pro-drug strategies may be used, e.g., conversion to a drug with increased oral availability.

a) Functional Nucleic Acids

Functional nucleic acids are nucleic acid molecules that have a specific function, such as binding a target molecule or catalyzing a specific reaction. For example, the functional nucleic acid can inhibit HIF-1α or any of the other enzymes that are part of the hypoxic pathway. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of HIF-1α or the genomic DNA of HIF-1α or they can interact with the polypeptide HIF-1α or a domain thereof, such as PAS-B. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d})less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

### 5. Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

### 6. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as those discussed above that inhibit HIF-1α, into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B 19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed nucleic acids or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### 7. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 8. Peptides

a) Protein Variants

As discussed herein there are numerous variants of the HIF-1α protein that are known and herein contemplated. In addition, to the known functional HIF-1α strain variants there are derivatives of the HIF-1α proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | Ala, A |
| arginine | Arg, R |
| asparagine | Asn, N |
| aspartic acid | Asp, D |
| cysteine | Cys, C |
| glutamic acid | Glu, E |
| glutamine | Gln, K |
| glycine | Gly, G |
| histidine | His, H |
| isolelucine | Ile, I |
| leucine | Leu, L |
| lysine | Lys, K |
| phenylalanine | Phe, F |
| proline | Pro, P |
| serine | Ser, S |
| threonine | Thr, T |
| tyrosine | Tyr, Y |
| tryptophan | Trp, W |
| valine | Val, V |

TABLE 2
Amino Acid Substitutions
Original Residue Exemplary Conservative Substitutions,
others are known in the art.

Ala.ser
Arg, lys, gln
Asn, gln; his
Asp, glu
Cys, ser
Gln, asn, lys
Glu, asp
Gly,, pro
His asn; gln
Ile, leu; val
Leu, ile; val
Lys, arg; gln;
Met, Leu; ile
Phe, met; leu; tyr
Ser, thr
Thr, ser
Trp, tyr
Tyr, trp; phe
Val, ile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appin, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### 9. Antibodies

(1) Antibodies Generally

The antibodies disclosed herein can be used as inhibitors of HIF-1α, or any of the enzymes in the hypoxic pathway. The term “antibodies” is used herein in a broad sense and includes both polyclonal and monoclonal antibodies. The antibodies can be tested for their desired activity using the in vitro assays described herein, or by analogous methods, after which their in vivo therapeutic and/or prophylactic activities are tested according to known clinical testing methods. The antibodies disclosed herein can be used to target the PAS-B domain of HIF-1α.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566, Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. Broadly neutralizing anti antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 10. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions disclosed herein, such as the HIF-1α inhibitors, may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing ischemic disease or tumor progression, the efficacy of the therapeutic antibody can be assessed in various ways well known to the skilled practitioner. For instance, one of ordinary skill in the art will understand that a composition, such as an antibody, disclosed herein is efficacious in treating or inhibiting an ischemic disease or tumor progression in a subject by observing that the composition reduces tumor progression or prevents a further increase in ischemia.

The compositions disclosed herein may be administered prophylactically to patients or subjects who are at risk for ischemic diseases or tumor progression. Other molecules that interact with HIF-1α which do not have a specific pharmaceutical function, but which may be used for tracking changes within cellular chromosomes or for the delivery of diagnostic tools for example can be delivered in ways similar to those described for the pharmaceutical products.

The disclosed compositions and methods can also be used for example as tools to isolate and test new drug candidates for a variety of hypoxia-related diseases.

### 11. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

### 12. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

### 13. Compositions Identified by Screening with Disclosed Compositions/Combinatorial Chemistry

a) Combinatorial Chemistry

The disclosed compositions can be used as targets for any combinatorial technique to identify molecules or macromolecular molecules that interact with the disclosed compositions in a desired way. Also disclosed are the compositions that are identified through combinatorial techniques or screening techniques in which HIF-1α or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, HIF-1α, are also disclosed. Thus, the products produced using the combinatorial or screening approaches that involve the disclosed compositions, such as, HIF-1α are also considered herein disclosed.

It is understood that the disclosed methods for identifying molecules that inhibit the interactions between, for example, HIF-1α PAS-B and nucleic acids can be performed using high through put means. For example, putative inhibitors can be identified using Fluorescence Resonance Energy Transfer (FRET) to quickly identify interactions. The underlying theory of the techniques is that when two molecules are close in space, ie, interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example the PAS-B domain of HIF-1α is attached to a DNA binding domain of a transcriptional activation protein, such as Gal 4. By performing the Two-hybrid technique on this type of system, molecules that bind the PAS-B domain of HIF-1α can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in interative processes.

b) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as HIF-1α, are also disclosed. Thus, the products produced using the molecular modeling approaches that involve the disclosed compositions, such as HIF-1α, and in particular the PAS-B region, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

### 14. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

### D. Methods of Making the Compositions

The compositions disclosed herein and the compositions necessary to perform the disclosed methods can be made using any method known to those of skill in the art for that particular reagent or compound unless otherwise specifically noted.

### 1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring

Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### 2. Peptide Synthesis

One method of producing the disclosed proteins, such as HIF-1α, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

### E. Methods of Using the Compositions

Provided herein is a method for preventing or reducing tumor growth, preferably metastatic tumor growth, in a subject in vivo, comprising administering to a subject in need thereof an effective amount of an inhibitor of HIF-1α activity; and optionally, a pharmaceutically acceptable carrier, thereby preventing or reducing tumor growth, for example by at least 25%, 50%, 75%, 90%, or 95%, in the subject treated. A detailed description of suitable compositions for use in the present treatment methods is given above. In particular, the method is useful when the tumor is hypoxic. Hypoxic tumors can be readily identified using routine methods in the art. See, e.g., U.S. Pat. No. 5,674,693.

Thus, provided herein is a method of treating metastasis in a subject with cancer in vivo, comprising administering to a subject in need thereof an effective amount of an inhibitor of HIF-1α activity, thereby inhibiting metastasis, for example, by at least 25%, 50%, 75%, 90%, or 95%, in the subject treated. Preferably, the inhibitor of HIF-1α specifically inhibits human HIF-1α, such as antibodies specifically binding to HIF-1α, not to other HIF-1α -like or HIF-1α -related proteins.

Also provided herein is a method of increasing or enhancing the chances of survival of a subject with metastatic tumor, comprising administering to a subject in need thereof an effective amount of an inhibitor of HIF-1α activity, thereby increasing or enhancing the chances of survival of the subject treated by a certain period of time, for example, by at least 10 days, 1 month, 3 months, 6 months, 1 year, 1.5 years, 2 years, 3 years, 4 years, 5 years, 8 years, or 10 years. The increase in survival of a subject can be defined, for example, as the increase in survival of a preclinical animal model of cancer metastases (e.g., a mouse with metastatic cancer), by a certain period of time, for example, by at least 10 days, 1 month, 3 months, 6 months, or 1 year, or at least 2 times, 3 times, 4 times, 5 times, 8 times, or 10 times, more than a control animal model (that has the same type of metastatic cancer) without the treatment with the inventive method. Optionally, the increase in survival of a mammal can also be defined, for example, as the increase in survival of a patient with cancer metastases by a certain period of time, for example, by at least 10 days, 1 month, 3 months, 6 months, 1 year, 1.5 years, 2 years, 3 years, 4 years, 5 years, 8 years, or 10 years more than a patient with the same type of metastatic cancer but without the treatment with the inventive method. The control patient may be on a placebo or treated with supportive standard care such as chemical therapy, biologics and/or radiation that do not include the inventive method as a part of the therapy.

Also provided herein is a method of stabilizing metastatic tumor burden of a subject, comprising administering to a subject in need thereof an effective amount of an inhibitor of HIF-1α activity, thereby stabilizing metastatic tumor burden of a subject for a certain period of time, for example, for at least 10 days, 1 month, 3 months, 6 months, 1 year, 1.5 years, 2 years, 3 years, 4 years, 5 years, 8 years, or 10 years. Stabilization of the metastatic tumor burden of a subject can be defined as stabilization of metastatic tumor burden of a preclinical animal model with metastatic tumor burden (e.g., a mouse with metastatic tumor) for a certain period of time, for example, for at least 10 days, 1 month, 3 months, 6 months, or 1 year more than a control animal model (that has the same type of metastatic tumor) without the treatment with the inventive method.

The present treatment methods also include a method to increase the efficacy of chemotherapeutic agents, comprising administering to a subject in need thereof an effective amount of an inhibitor of HIF-1α activity; and optionally, a pharmaceutically acceptable carrier, thereby increasing the efficacy of chemotherapeutic agents. Also contemplated are methods involving the delivery of HIF-1α inhibitory formulations in combination with radiation therapy. Radiation therapy may be used to treat almost every type of solid tumor, including cancers of the brain, breast, cervix, larynx, lung, pancreas, prostate, skin, spine, stomach, uterus, or soft tissue sarcomas. Radiation can also be used to treat leukemia and lymphoma (cancers of the blood-forming cells and lymphatic system, respectively). Radiation dose to each site depends on a number of factors, including the type of cancer and whether there are tissues and organs nearby that may be damaged by radiation. The radiation will typically be delivered as X-rays, where the dosage is dependent on the tissue being treated. Radiopharmaceuticals, also known as radionucleotides, may also be used to treat cancer, including thyroid cancer, cancer that recurs in the chest wall, and pain caused by the spread of cancer to the bone (bone metastases).

The subject treated or diagnosed by the present methods includes a subject having or being at risk of having metastatic tumor growth. Such tumors can be a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. Tumors treated by compounds of the present methods include, but are not limited to: neoplasm of the central nervous system: glioblastomamultiforme, astrocytoma, oligodendroglial tumors, ependymal and choroids plexus tumors, pineal tumors, neuronal tumors, medulloblastoma, schwannoma, meningioma, meningeal sarcoma: neoplasm of the eye: basal cell carcinoma, squamous cell carcinoma, melanoma, rhabdomyosarcoma, retinoblastoma; neoplasm of the endocrine glands: pituitary neoplasms, neoplasms of the thyroid, neoplasms of the adrenal cortex, neoplasms of the neuroendocrine system, neoplasms of the gastroenteropancreatic endocrine system, neoplasms of the gonads; neoplasms of the head and neck: head and neck cancer, oral cavity, pharynx, larynx, odontogenic tumors: neoplasms of the thorax: large cell lung carcinoma, small cell lung carcinoma, non-small cell lung carcinoma, neoplasms of the thorax, malignant mesothelioma, thymomas, primary germ cell tumors of the thorax; neoplasms of the alimentary canal: neoplasms of the esophagus, neoplasms of the stomach, neoplasms of the liver, neoplasms of the gallbladder, neoplasms of the exocrine pancreas, neoplasms of the small intestine, vermiform appendix and peritoneum, adenocarcinoma of the colon and rectum, neoplasms of the anus; neoplasms of the genitourinary tract: renal cell carcinoma, neoplasms of the renal pelvis and ureter, neoplasms of the bladder, neoplasms of the urethra, neoplasms of the prostate, neoplasms of the penis, neoplasms of the testis; neoplasms of the female reproductive organs: neoplasms of the vulva and vagina, neoplasms of the cervix, adenocarcinoma of the uterine corpus, ovarian cancer, gynecologic sarcomas; neoplasms of the breast; neoplasms of the skin: basal cell carcinoma, squamous carcinoma, dermatofibrosarcoma, Merkel cell tumor; malignant melanoma; neoplasms of the bone and soft tissue: osteogenic sarcoma, malignant fibrous histiocytoma, chrondrosarcoma, Ewing's sarcoma, primitive neuroectodermal tumor, angiosarcoma; neoplasms of the hematopoietic system: myelodysplastic syndromes, acute myeloid leukemia, chronic myeloid leukemia, acute lymphocytic leukemia, HTLV-1, and T-cell leukemia/lymphoma, chronic lymphocytic leukemia, hairy cell leukemia, Hodgkin's disease, non-Hodgkin's lymphomas, mast cell leukemia; neoplasms of children: acute lymphoblastic leukemia, acute myelocytic leukemias, neuroblastoma, bone tumors, rhabdomyosarcoma, lymphomas, renal and liver tumors. In certain embodiment, the tumor is a breast tumor, a pancreas tumor, a lung tumor, a cervical tumor, a colon tumor or a head and neck tumor.

The present invention also provides a method for preventing or reducing the risk of tumor metastasis in a subject, comprising administering to a subject in need thereof an effective amount of an inhibitor of HIF-1α activity; and optionally, a pharmaceutically acceptable carrier, thereby preventing or reducing preventing or reducing the risk of tumor metastasis. The inhibitor can be a peptide, an antibody, a pharmacological inhibitor, siRNA, snRNA or antisense nucleic acid. The subject in need of such a prophylactic may be an individual who is genetically predisposed to cancer or at a high risk of developing cancer due to various reasons such as family history of cancer and carcinogenic environment.

Examples of the human gene that is involved in the onset or development of cancer include, but are not limited to, VHL (the Von Hippon Landau gene involved in Renal Cell Carcinoma); P16/INK4A (involved in lymphoma); E-cadherin (involved in metastasis of breast, thyroid, gastric cancer); hMLH1 (involved in DNA repair in colon, gastric, and endometrial cancer); BRCA1 (involved in DNA repair in breast and ovarian cancer); LKB1 (involved in colon and breast cancer); P15/INK4B (involved in leukemia such as AML and ALL); ER (estrogen receptor, involved in breast, colon cancer and leukemia); O6-MGMT (involved in DNA repair in brain, colon, lung cancer and lymphoma); GST-pi (involved in breast, prostate, and renal cancer); TIMP-3 (tissue metalloprotease, involved in colon, renal, and brain cancer metastasis); DAPK1 (DAP kinase, involved in apoptosis of B-cell lymphoma cells); P73 (involved in apoptosis of lymphomas cells); AR (androgen receptor, involved in prostate cancer); RAR-beta (retinoic acid receptor-beta, involved in prostate cancer); Endothelin-B receptor (involved in prostate cancer); Rb (involved in cell cycle regulation of retinoblastoma); p53 (an important tumor suppressor gene); P14ARF (involved in cell cycle regulation); RASSF1 (involved in signal transduction); APC (involved in signal transduction); Caspase-8 (involved in apoptosis); TERT (involved in senescence); TERC (involved in senescence); TMS-1 (involved in apoptosis); SOCS-1 (involved in growth factor response of hepatocarcinoma); PITX2 (hepatocarcinoma breast cancer); MINT1; MINT2; GPR37; SDC4; MYOD1; MDR1; THBS1; PTC1; and pMDR1, as described in Santini et al. (2001) Ann. of Intern. Med. 134:573-586, which is herein incorporated by reference in its entirety. Nucleotide sequences of these genes can be retrieved from the website of the National Center for Biotechnology Information (NCBI).

Staging of solid tumor cancers is well known. The TNM system is one of the most commonly used staging systems. This system has been accepted by the International Union Against Cancer (UICC) and the American Joint Committee on Cancer (AJCC). Most medical facilities use the TNM system as their main method for cancer reporting. PDQ®, the NCI's comprehensive cancer database, also uses the TNM system.

The TNM system, referred to herein as “staging,” is based on the extent of the tumor, the extent of spread to the lymph nodes, and the presence of metastasis.

As to diagnostic methods, the screening or diagnostic analysis of patient samples can be performed in order to determine HIF-1α levels and, accordingly metastatic aggressiveness of tumors. This analysis may be performed prior to the initiation of treatment using HIF-1α-specific therapy to identify tumors having elevated HIF-1α expression or activity. Such diagnosis analysis can be performed using any sample, including but not limited to cells, protein or membrane extracts of cells, biological fluids such as sputum, blood, serum, plasma, or urine, or biological samples such as formalin-fixed or frozen tissue sections employing the antibodies of the present invention. Any suitable method for detection and analysis of HIF-1α expression can be employed. As used herein, the term “sample” refers to a sample from a human, animal, or to a research sample, e.g., a cell, tissue, organ, fluid, gas, aerosol, slurry, colloid, or coagulated material. The sample may be tested in vivo, e.g., without removal from the human or animal, or it may be tested in vitro. The sample may be tested after processing, e.g., by histological methods. The term “sample” may also refer to a cell, tissue, organ, or fluid that is freshly taken from a human or animal, or to a cell, tissue, organ, or fluid that is processed or stored.

Also provided herein is a method for staging tumor growth or metastasis in a subject, comprising assessing the HIF-1α levels in a tumor of the subject, whereby a change in HIF-1α level (e.g., in gene expression or enzymatic activity) in the tumor in comparison with a reference sample, indicates the presence of metastatic tumor growth. In some instances, the HIF-1α levels or activities in the tumor may be higher than those when measured earlier for the same subject, or higher than those in a reference sample taken from a normal tissue, which may indicate that the patient is at a greater risk of tumor metastasis; that the tumor has metastasized; or that tumor metastasis has increased.

Also provided herein is a method for diagnosing cancer metastasis in a subject, comprising assessing the HIF-1α levels in the blood, whereby a change HIF-1α level (e.g., in gene expression or enzymatic activity) in the blood in comparison with a reference sample, indicates the presence of metastatic tumor growth. In some instances, the HIF-1α levels or activities in the blood may be lower than those when measured earlier, which may indicate that the patient is at a greater risk of cancer metastasis; that the cancer has metastasized; or that cancer metastasis has increased. The reference sample may derive from the same subject, taken from the same tumor at a different time point or from other site of the body, or from another individual.

Measurement of HIF-1α levels may take the form of an immunological assay, which detects the presence of a HIF-1α protein with an antibody to the protein, preferably an antibody specifically binding to HIF-1α. Such assays for other proteins are well known, and may be adapted to detection of HIF-1α proteins. Immunoassays also can be used in conjunction with laser induced fluorescence (see, for example, Schmalzing and Nashabeh, Electrophoresis 18:2184-93 (1997)); Bao, J. Chromatogr. B. Biomed. Sci. 699:463-80 (1997), each of which is incorporated herein by reference). Liposome immunoassays, such as flow-injection liposome immunoassays and liposome immunosensors, also can be used to determine HIF-1α levels according to a method of the invention (Rongen et al., J. Immunol. Methods 204:105-133 (1997), which is incorporated by reference herein). Immunoassays, such as enzyme-linked immunosorbent assays (ELISAs), can be particularly useful in a method of the invention. A radioimmunoassay also can be useful for determining whether a sample is positive for HIF-1α or for determining the level of HIF-1α. A radioimmunoassay using, for example, an iodine-125 labeled secondary antibody, may be used.

Disclosed herein is a method of increasing DNA repair in a cell, comprising contacting the cell with an inhibitor of a component of the Myc pathway. The HIF-1α-Myc pathway involves Myc displacement from the CDNK1A promoter by the hypoxia-inducible transcription factor HIF-1α, (Example 3). An example of a component of the Myc pathway is Sp1, and another is HIF-1α itself. DNA repair can be increased by decreasing interference of DNA repair enzymes. DNA repair can be increased by 10, 20, 30, 40, 50, 60, 70, 80, or 90% or more compared to a control.

Also disclosed is a method of treating a subject with cancer, comprising administering to the subject an effective amount of an inhibitor of a component of the Myc pathway, along with antiangiogenesis treatment. The antiangiogenesis treatment can be, for example, ADH-1 (Exherin™), AG-013736, AMG-706, Anti-VEGF Antibody (Bevacizumab; Avastin™), AZD2171, Bay 43-9006 (Sorafenib tosylate), BMS-582664, CHIR-265, GW786034 (Pazopanib), PI-88, PTK787/ZK 222584 (Vatalanib), RAD001 (Everolimus), Suramin, SU11248 (Sunitinib malate), XL184, and ZD6474.

Also disclosed herein a nucleic acid molecule encoding a polypeptide comprising PAS-B of a hypoxia inducible factor, wherein the PAS-B comprises at least one mutation which differs from naturally occurring PAS-B of a hypoxia inducible factor. As discussed herein, the hypoxia inducible factor can be HIF-1α. Also disclosed is an expression vector comprising a nucleic acid molecule as described herein, operatively linked to an expression control sequence. The expression control sequence can comprise an inducible promoter. In one embodiment, the vector can be an adenoviral vector. Also disclosed is a host cell comprising an expression vector as described herein.

Further disclosed is a polypeptide comprising a PAS-B of a hypoxia inducible factor, wherein the PAS-B comprises at least one mutation which differs from naturally occurring PAS-B of a hypoxia inducible factor. Also disclosed is a pharmaceutical composition comprising an expression vector according and a pharmaceutically acceptable carrier.

Disclosed herein is a method for increasing the expression in a target cell of a hypoxia-inducible gene, said method comprising the steps of (a) introducing into said cell an expression vector as disclosed herein; and (b) allowing expression of said protein encoded by said expression vector.

Also disclosed is a method for providing sustained expression of biologically active HIF-1α in a cell under normoxic conditions, said method comprising the step of introducing into said cell a nucleic acid molecule as disclosed herein, operatively linked to an expression control sequence which directs its expression in said cell.

Further disclosed is a method for reducing ischemic tissue damage in a subject having a hypoxia-associated disorder comprising the steps of administering to said subject an effective amount of a pharmaceutical composition described herein.

Disclosed is a method for reducing ischemic tissue damage in a subject having a hypoxia-associated disorder comprising the steps of: (a) isolating cells to be implanted into said subject (b) introducing into said cells an expression vector disclosed herein; and (c) implanting said cells containing said expression vector into said subject.

Also disclosed is a method of treating cancer in a subject in need thereof, comprising administering an effective amount of a HIF-1α PAS-B inhibitor or a mutant PAS-B, wherein the cancer is a HIF-1α expressing cancer. The cancer can be selected from the group consisting of cervical cancer, lung cancer, breast cancer, oligodendroglioma, orpharyngeal squamous cell carcinoma, ovarian cancer, oesophageal cancer, endometrial cancer, head and neck cancer, human lung carcinoma, human colon carcinoma, pancreatic cancer, prostate cancer and gastrointestinal stromal tumor of the stomach. More on methods of treating cancer can be found below.

Also disclosed is a method for treating an HIF-1-mediated disorder in a subject comprising administering an effective amount of an HIF-1α PAS-B inhibitor or a mutant PAS-B. The HIF-1α PAS-B inhibitor or a mutant PAS-B can reduce the level of expression of HIF-1α. The level of expression of HIF-1α can be reduced by at least 10%, 205, 30%, 40%, 50%, 0%, 70%, 80%, 90%, or 100%.

Also disclosed is a method of screening for a test compound that modulates HIF-1α PAS-B, comprising: contacting HIF-1α PAS-B with a test compound; detecting interaction between HIF-1α PAS-B and the test compound; wherein interaction between HIF-1α PAS-B and the test compound indicates a test compound that modulates HIF-1α PAS-B. The ability to modulate HIF-1α PAS-B can be measured by contacting the test compound with one or more tumor cells. Furthermore, a plurality of test compounds can be contacted with HIF-1α PAS-B in a high throughput assay system. The high throughput assay system can comprise an immobilized array of test compounds. The high throughput assay system can also comprise an immobilized array of HIF-1α PAS-B molecules. Lastly, disclosed are compounds identified by these methods.

Disclosed herein are methods for the treatment, prevention, and/or management of diseases or disorders associated with overexpression of HIF-1α and/or increased HIF-1α activity (e.g., cancer, respiratory disorders such as asthma and ischemic diseases). In particular, disclosed are methods of manipulating the PAS-B region of HIF-1α. In one embodiment, the PAS-B region can be mutated. Without being bound to a particular mechanism of action, administration of inhibitors or mutated versions of PAS-B can exert protective effects against hypoxia and renders those cells susceptible to destruction due to hypoxia.

The methods and compositions of the invention comprising HIF-1α PAS-B inhibitors or PAS-B mutants are particularly useful when the levels of HIF-1α expression and/or activity are elevated above the standard or background level, as determined using methods known to those skilled in the art and disclosed herein. As used herein, “elevation” of a measured level of HIF-1α relative to a standard level means that the amount or concentration of HIF-1α in a sample or subject is sufficiently greater in a subject or sample relative to the standard as detected by any method now known in the art or to be developed in the future for measuring HIF-1α levels. For example, elevation of the measured level relative to a standard level may be any statistically significant elevation detectable. Such an elevation in HIF-1α expression and/or activity may include, but is not limited to about a 10%, about a 20%, about a 40%, about an 80%, about a 2-fold, about a 4-fold, about an 10-fold, about a 20-fold, about a 50-fold, about a 100-fold, about a 2 to 20 fold, 2 to 50 fold, 2 to 100 fold, 20 to 50 fold, 20 to 100 fold, elevation, relative to the standard. The term “about” as used in this context refers to levels of elevation of the standard numerical value plus or minus 10% of the numerical value.

The term “standard level” or “background level” as used herein refers to a baseline amount of HIF-1α level as determined in one or more normal subjects, i.e., a subject with no known history of past or current diseases, disorders or cancer. For example, a baseline may be obtained from at least one subject and preferably is obtained from an average of subjects (e.g., n=2 to 100 or more), wherein the subject or subjects have no prior history of diseases, disorders or cancer, especially no prior history of diseases associated with an aberrant level of expression and/or activity of HIF-1α.

In a preferred specific embodiment, the invention encompasses a method for treatment, prevention and/or management of diseases or disorders associated with overexpression of HIF-1α and/or increased HIF-1α activity (e.g., cancer, respiratory disorders such as asthma and obstructive pulmonary disorders or ischemic disease) comprising administering a therapeutically and/or prophylactically effective amount of HIF-1α PAS-B mutant or inhibitor as disclosed herein.

Prophylactic and therapeutic compounds that may be used in the methods and compositions of the invention include, but are not limited to, proteinaceous molecules, including, but not limited to, peptides, polypeptides, proteins, including post-translationally modified proteins, antibodies, etc.; small molecules (less than 1000 daltons), inorganic or organic compounds; nucleic acid molecules including, but not limited to, double-stranded or single-stranded DNA, double-stranded or single-stranded RNA, as well as triple helix nucleic acid molecules. Prophylactic and therapeutic compounds can be derived from any known organism (including, but not limited to, animals, plants, bacteria, fungi, and protista, or viruses) or from a library of synthetic molecules. In certain embodiments, one or more compounds of the invention are administered to a mammal, preferably a human, concurrently with one or more other therapeutic agents useful for the treatment of cancer or a disorder. The term “concurrently” is not limited to the administration of prophylactic or therapeutic agents at exactly the same time, but rather it is meant that compounds of the invention and the other agent are administered to a subject in a sequence and within a time interval such that the compounds of the invention can act together with the other agent to provide an increased benefit than if they were administered otherwise. For example, each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In various embodiments, the prophylactic or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

In other embodiments, the present invention encompasses methods for treatment, prevention and/or management of cancer or other diseases comprising administering a PAS-B mutant or inhibitor in combination with other therapeutic and/or prophylactic agents. These can be used for the treatment of ischemic diseases, or to treat cancer, e.g., with cytotoxic agents. The combination therapies disclosed herein can work by attacking the tumor cell directly, inhibit growth of new blood vessels around the tumor cell, and, by virtue of the HIF-1α PAS-B inhibitors or mutants, inhibit the ability of the cell to survive without the growth of new blood vessels. The compositions disclosed herein can be used for enhancing chemotherapeutic treatment of cancers and countering multi-drug resistance in cancers. The HIF-1α PAS-B inhibitors and mutants can be administered before or during administration of a taxane family, vinca alkaloid, camptothecin or antibiotic compound.

### 1. Methods of Treating Asthma

The methods and compositions comprising HIF-1α PAS-B mutants or inhibitors of the invention are effective for treatment, prevention, and/or management of asthma. The therapeutic and prophylactic methods of the invention for asthma may be used in combination with other methods known in the art for the treatment, prevention and/or management of asthma including but not limited to inhaled beta 2 agonists, inhaled corticosteroids, retinoic acid, anti-IgE antibodies, phosphodiesterase inhibitors, leukotriene antagonists, anti IL-9 antibody, and/or anti-mucin therapies (e.g., anti hCLCA1 therapy such as Lomucin™). .beta.-adrenergic drugs (e.g. epinephrine and isoproterenol), theophylline, anticholinergic drugs (e.g., atropine and ipratorpium bromide), and corticosteroids, adrenergic stimulants (e.g., catecholamines (e.g., epinephrine, isoproterenol, and isoetharine), resorcinols (e.g., metaproterenol, terbutaline, and fenoterol), and saligenins (e.g., salbutamol), other steroids, immunosuppressant agents (e.g., methotrexate and gold salts), mast cell modulators (e.g., cromolyn sodium (INTAL™) and nedocromil sodium (TILADE™)), and mucolytic agents (e.g., acetylcysteine)).

### 2. Methods of Treating Ischemia

In one aspect, the invention provides methods for treating various ischemic and hypoxic conditions, in particular, using the compounds described herein. In one embodiment, the methods of the invention produce therapeutic benefit when administered following ischemia or hypoxia. For example, the methods of the invention produce a dramatic decrease in morbidity and mortality following myocardial infarction, and a significant improvement in heart architecture and performance. Further, the methods of the invention improve liver function when administered following hepatic toxic-ischemic injury. Hypoxia is a significant component of liver disease, especially in chronic liver disease associated with hepatotoxic compounds such as ethanol. Additionally, expression of genes known to be induced by HIF-1α, e.g., nitric oxide synthase and glucose transporter-1, is increased in alcoholic liver disease. (See, e.g., Areel et al. (1997) Hepatology 25:920-926; Strubelt (1984) Fundam Appl Toxicol 4:144-151; Sato (1983) Pharmacol Biochem Behav 18 (Suppl 1):443-447; Nanji et al. (1995) Am J Pathol 146:329-334; and Morio et al. (2001) Toxicol Appl Pharmacol 172:44-51.)

Therefore, the present invention provides methods of treating conditions associated with ischemia or hypoxia, the method comprising administering a therapeutically effective amount of a compound or a pharmaceutically acceptable salt thereof, alone or in combination with a pharmaceutically acceptable excipient, to a subject. In one embodiment, the compound is administered immediately following a condition producing acute ischemia, e.g., myocardial infarction, pulmonary embolism, intestinal infarction, ischemic stroke, and renal ischemic-reperfusion injury. In another embodiment, the compound is administered to a patient diagnosed with a condition associated with the development of chronic ischemia, e.g., cardiac cirrhosis, macular degeneration, pulmonary embolism, acute respiratory failure, neonatal respiratory distress syndrome, and congestive heart failure. In yet another embodiment, the compound is administered immediately after a trauma or injury.

In another aspect, the invention provides methods for treating a patient at risk of developing an ischemic or hypoxic condition, e.g., individuals at high risk for atherosclerosis, etc., using the compounds described herein. Risk factors for atherosclerosis include, e.g., hyperlipidemia, cigarette smoking, hypertension, diabetes mellitus, hyperinsulinemia, and abdominal obesity. Therefore, the present invention provides methods of preventing ischemic tissue injury, the method comprising administering a therapeutically effective amount of a compound or a pharmaceutically acceptable salt thereof; alone or in combination with a pharmaceutically acceptable excipient, to a patient in need. In one embodiment, the compound can be administered based on predisposing conditions, e.g., hypertension, diabetes, occlusive arterial disease, chronic venous insufficiency, Raynaud's disease, chronic skin ulcers, cirrhosis, congestive heart failure, and systemic sclerosis.

In one specific embodiment, the methods are used to increase vascularization and/or granulation tissue formation in damaged tissue, wounds, and ulcers. For example, compounds of the invention have been shown to be effective in stimulating granulation tissue formation in wound healing. Granulation tissue contains newly formed, leaky blood vessels and a provisional stroma of plasma proteins, such as fibrinogen and plasma fibronectin. Release of growth factors from inflammatory cells, platelets, and activated endothelium, stimulates fibroblast and endothelial cell migration and proliferation within the granulation tissue. Ulceration can occur if vascularization or neuronal stimulation is impaired. The methods of the invention are effective at promoting granulation tissue formation. Thus, the invention provides methods for treating a patient having tissue damage due to, e.g., an infarct, having wounds induced by, e.g., trauma or injury, or having chronic wounds or ulcers produced as a consequence of a disorder, e.g., diabetes. The method comprises administering a therapeutically effective amount of a compound or a pharmaceutically acceptable salt thereof, alone or in combination with a pharmaceutically acceptable excipient, to a patient in need.

In another aspect, the invention provides methods of using the compounds to pretreat a subject to decrease or prevent the development of tissue damage associated with ischemia or hypoxia. The methods of the invention produce therapeutic benefit when administered immediately before a condition involving ischemia or hypoxia. For example, application of the methods of the invention prior to induction of myocardial infarction shows statistically significant improvement in heart architecture and performance. Further, the methods of the invention produce therapeutic benefit when administered immediately before and during ischemic-reperfusion injury, significantly reducing diagnostic parameters associated with renal failure.

Therefore, the invention provides methods of pretreating a subject to decrease or prevent the tissue damage associated with ischemia or hypoxia, the method comprising administering a therapeutically effective amount of a compound or a pharmaceutically acceptable salt thereof, alone or in combination with a pharmaceutically acceptable excipient, to a patient with a history of ischemic disorders, e.g., myocardial infarctions, or having symptoms of impending ischemia, e.g., angina pectoris. In another embodiment, the compound can be administered based on physical parameters implicating possible ischemia, e.g., individuals placed under general anesthesia or temporarily working at high altitudes. In yet another embodiment, the compounds may be used in organ transplants to pretreat organ donors and to maintain organs removed from the body prior to implantation in the recipient.

The methods of treatment disclosed herein can be administered in combination with various other therapeutic approaches. In one embodiment, the compound is administered with a 2-oxoglutarate dioxygenase inhibitor. In another embodiment, the compound is administered with another therapeutic agent having a different mode of action, e.g., an ACE inhibitor (ACEI), angiotensin-II receptor blocker (ARB), statin, diuretic, digoxin, carnitine, etc.

### 3. Method of Treating Cancer

As used herein the term “HIF-1α expressing cancers” refers to human cancer that express HIF-1α or that otherwise comprise elevated concentrations of HIF-1α. Elevated concentration is determined by comparison to normal, non-cancerous tissues of a similar cell type. Examples of HIF-1α expressing cancers include without limitation cervical cancer (early stage), lung cancer (non-small cell lung carcinoma), breast cancer (including lymph node positive breast cancer and lymph node negative breast cancer), oligodendroglioma, orpharyngeal squamous cell carcinoma, ovarian cancer, oesophageal cancer, endometrial cancer, head and neck cancer, gastrointestinal stromal tumor of the stomach.

The methods and compositions of the invention comprising HIF-1α PAS-B mutants and inhibitors are particularly useful for the treatment, inhibition or regression of solid tumors. As used herein, “solid tumors” refer to a locus of tumor cells where the majority of the cells are tumor cells or tumor-associated cells, including but not limited to laryngeal tumors, brain tumors, and other tumors of the head and neck; colon, rectal and prostate tumors; breast and thoracic solid tumors; ovarian and uterine tumors; tumors of the esophagus, stomach, pancreas and liver; bladder and gall bladder tumors; testicular cancer; skin tumors such as melanomas. Moreover, the tumors encompassed within the invention can be either primary or a secondary tumor resulting from metastasis of cancer cells elsewhere in the body to the chest.

In particular embodiments, the methods and compositions of the invention comprise the administration of one or more HIF-1α PAS-B mutants or inhibitors (alone or in combination with other anti-cancer agents) to subjects/patients suffering from or expected to suffer from cancer, e.g., have a genetic predisposition for a particular type of cancer, have been exposed to a carcinogen, or are in remission from a particular cancer. The methods and compositions of the invention may be used as a first line or second line cancer treatment. Included in the invention is also the treatment of patients undergoing other cancer therapies and the methods and compositions of the invention can be used before any adverse effects or intolerance of these other cancer therapies occurs. The invention also encompasses methods for administering one or more compounds of the invention to treat or ameliorate symptoms in refractory patients. In a certain embodiment, that a cancer is refractory to a therapy means that at least some significant portion of the cancer cells are not killed or their cell division arrested. The determination of whether the cancer cells are refractory can be made either in vivo or in vitro by any method known in the art for assaying the effectiveness of treatment on cancer cells, using the art-accepted meanings of “refractory” in such a context. In various embodiments, a cancer is refractory where the number of cancer cells has not been significantly reduced, or has increased. Also disclosed are methods for administering one or more HIF-1α PAS-B mutants or inhibitors to prevent the onset or recurrence of cancer in patients predisposed to having cancer.

In alternate embodiments, disclosed herein are methods for treating cancer in a subject by administering one or more HIF-1α PAS-B mutants or inhibitors in combination with any other treatment or to patients who have proven refractory to other treatments but are no longer on these treatments. In certain embodiments, the patients being treated by the methods of the invention are patients already being treated with chemotherapy, radiation therapy, hormonal therapy, or biological therapy/immunotherapy. Among these patients are refractory patients and those with cancer despite treatment with existing cancer therapies. In other embodiments, the patients have been treated and have no disease activity and one or more HIF-1α PAS-B mutants or inhibitors of the invention are administered to prevent the recurrence of cancer.

Additionally, disclosed herein are methods of treatment of cancer as an alternative to chemotherapy, radiation therapy, hormonal therapy, and/or biological therapy/immunotherapy where the therapy has proven or may prove too toxic, i.e., results in unacceptable or unbearable side effects, for the subject being treated. The subject being treated with the methods of the invention may, optionally, be treated with other cancer treatments such as surgery, chemotherapy, radiation therapy, hormonal therapy or biological therapy, depending on which treatment was found to be unacceptable or unbearable.

Chemotherapeutic cancer agents that can be used in combination with those disclosed herein include, but are not limited to, mitotic inhibitors (vinca alkaloids). These include vincristine, vinblastine, vindesine and Navelbine™ (vinorelbine,5′-noranhydroblastine). In yet other embodiments, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that may be used in the methods and compositions of the invention are podophyllotoxin derivatives, such as etoposide, teniposide and mitopodozide. The invention further encompasses other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The invention encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions of the invention include antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The invention further encompasses other chemotherapeutic cancer agents including without limitation anti-tumor antibodies, dacarbazine, azacytidine, amsacrine, melphalan, ifosfamide and mitoxantrone.

The compositions disclosed herein can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the invention include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with HIF-1α PAS-B mutants or inhibitors, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfm; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

The method can further comprise the administration of one or more additional cancer therapies. The additional cancer therapy can be selected from the group consisting of chemotherapy, immunotherapy, radiation therapy, hormonal therapy, or surgery. Furthermore, one or more anti-cancer agents can also be administered.

The anti-cancer agent can be selected from the group consisting of a chemotherapeutic agent, an anti-angiogenic agent, a cytotoxic agent and a cancer therapeutic antibody.

Also disclosed is a method of inhibiting the growth of a solid hypoxic tumor in a subject, comprising administering an effective amount of an HIF-1α PAS-B inhibitor or a mutant PAS-B. The growth of the solid hypoxic tumor can be reduced by at least 10%, 20%, 30%, 40%, 50%, 60%, 70%, 80%, 90%, or 100%.

The disclosed compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the treatment of precancer conditions such as cervical and anal dysplasias, other dysplasias, severe dysplasias, hyperplasias, atypical hyperplasias, and neoplasias.

### 4. Gene Disruption/Modification

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination event occur at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

### F. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

a) HIF-1α, Distinct from HIF-2, is Critical for NBS1 Downregulation by Hypoxia.

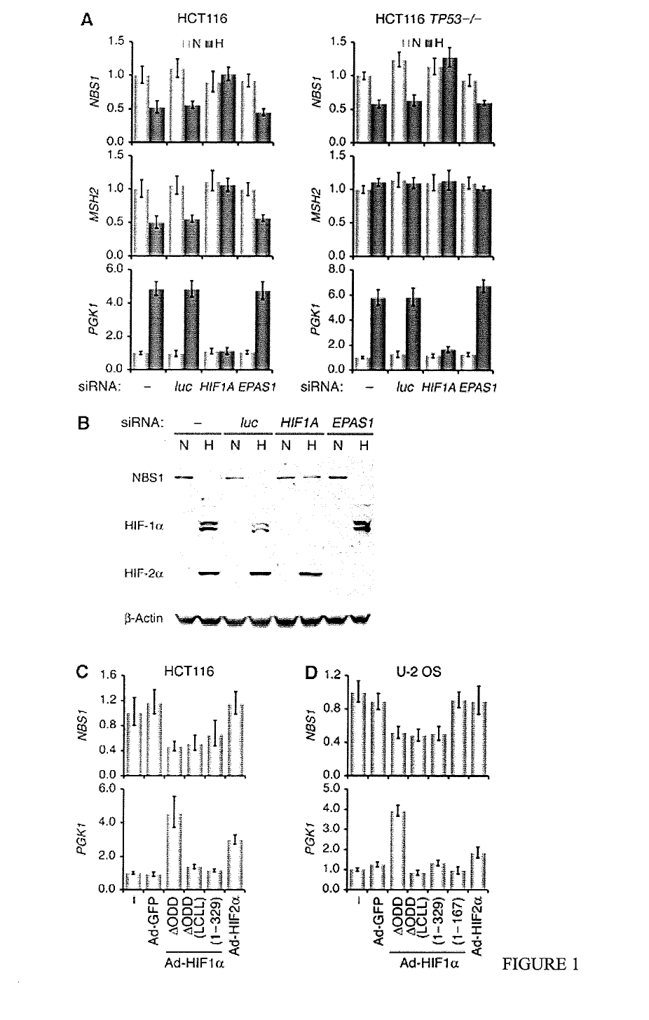

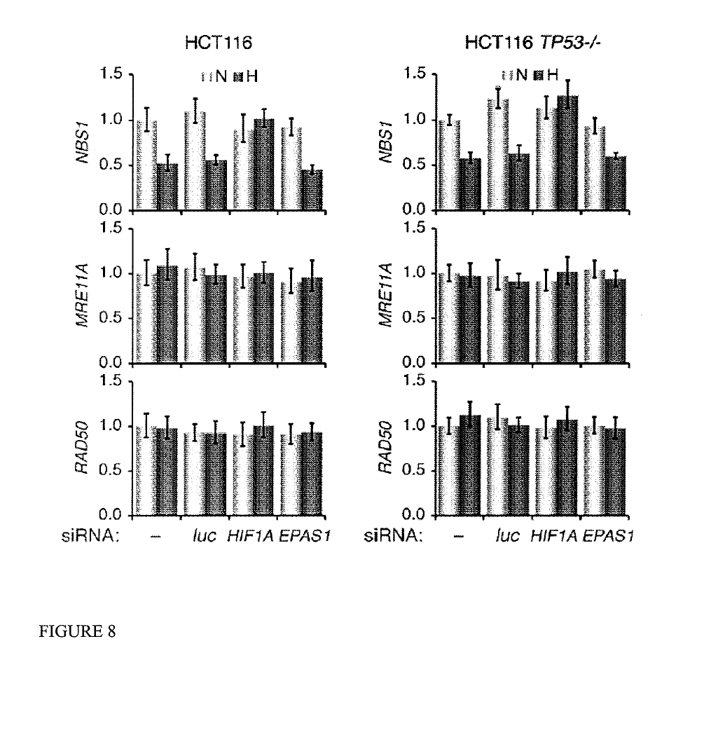

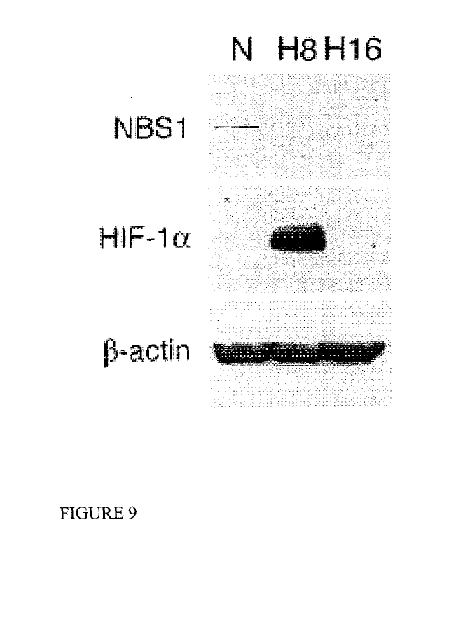

In pursuit of the involvement of HIF-α in chromosomal instability, the investigation focused on the expression of the MRN complex. Results from real-time PCR showed that NBS1 mRNA levels in HCT116 cells were reduced by 50% after a 16-hour hypoxic treatment (1% O2) (FIG. 1A), similar to the MSH2 repression. However, MRE11A and RAD50 expression were unaffected by the treatment (FIG. 8). Interestingly, similar NBS1 downregulation was observed in HCT116 TP53−/− cells, in stark contrast to the strict p53-dependence for MSH2 inhibition (Koshiji et al, 2005). Again, neither MRE11A nor RAD50 was inhibited in these cells, indicating specific NBS1 inhibition by a p53-independent mechanism. Furthermore, NBS1 protein levels were markedly decreased after 8- and 16-h hypoxic treatment (FIG. 9).

To test the requirement of HIF-α for NBS1 inhibition, small interfering RNA (siRNA) targeting HIF1A and EPAS1 (encoding HIF-2α) was used. In FIG. 1A, HIF1A siRNA abrogated the NBS1 and MSH2 downregulation, as well as the PGK1 upregulation in hypoxic cells, whereas EPAS 1 siRNA showed no obvious effects. Likewise, NBS1 protein levels remained equivalent in hypoxic U-2 OS cells when HIF-1α protein expression was abolished (FIG. 1B). Collectively, these findings suggest that HIF-1α, but not HIF-2α, is required for NBS1 repression by hypoxia.

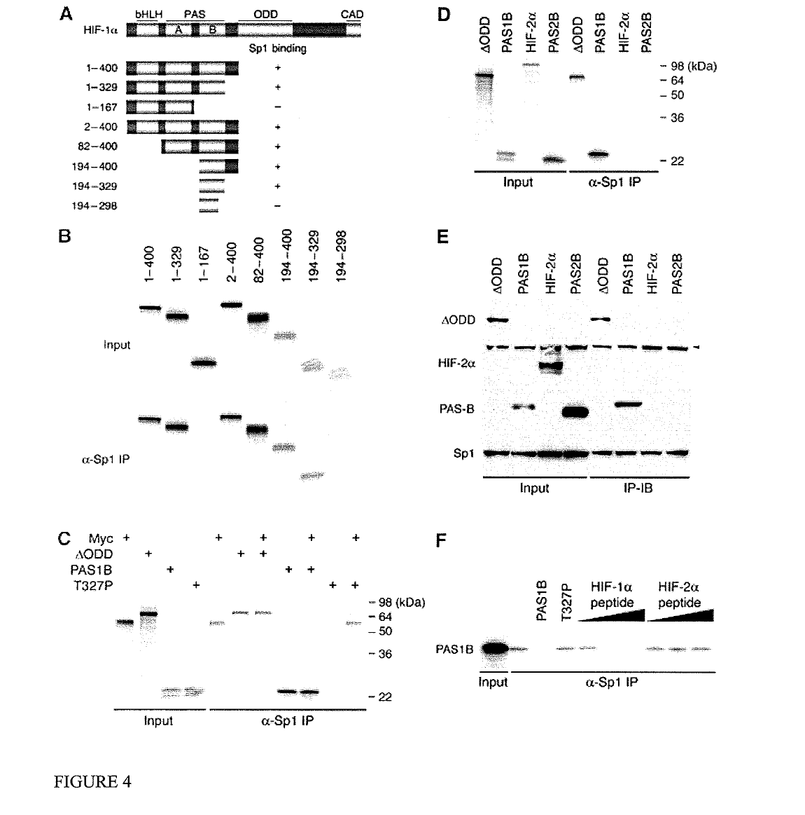

Next, it was asked whether HIF-1α is sufficient to inhibit NBS1 expression by infecting HCT116 cells with recombinant adenoviruses expressing a stable HIF-1α (Ad-HIF1αΔODD) (Huang et al, 1998; Koshiji et al, 2004). Results in FIG. 1C show that forced expression of HIF1αΔODD reduced NBS1 mRNA levels by 60%. More importantly, two HIF-1α variants lacking functional transactivation domains, Ad-HIF1αΔODD (LCLL) (Gu et al, 2001; Koshiji et al, 2004) and Ad-HIF1 (1-329) (see FIG. 4A), were also effective in NBS1 repression. By contrast, no inhibition of NBS1 was observed with the forced expression of HIF-2α, even though PGK1 was upregulated. Likewise, overexpression of the three HIF-1α variants in U-2 OS cells also lowered NBS1 mRNA levels by about 50% (FIG. 1D), but not with Ad-HIF1α (1-167) devoid of PAS-B (see FIG. 4A). Similarly, Ad-HIF2α showed no obvious effect on NBS1 expression, but markedly stimulated the expression of HIF-2α specific target. It should be noted that although during viral replication adenoviral E4 inactivates the MRN complex (Stracker et al, 2002), the recombinant adenoviruses used here are replication-defective, and the results show specific inhibition of NBS1 by HIF-1α. Therefore, it was concluded that HIF-1α, especially its N-terminal portion, is sufficient to mediate NBS1 repression by hypoxia.

b) Hypoxic Repression of NBS1 is Associated with the Induction of DNA DSBs.

To ascertain the functional relevance of NBS1 repression by hypoxia, the extent of DNA damage was assessed by immunofluorescent staining of γ-H2AX foci, a most sensitive method for quantifying DNA DSBs (Rogakou et al, 1998, 1999). In normoxia, U-2 OS cells displayed an average of two γ-H2AX foci per cell, whereas 72-h hypoxic treatment markedly increased the number by 3-fold (FIG. 9; Table 3). In particular, some of the hypoxic nuclei exhibited numerous, intensified γ-H2AX foci. Cells treated with desferrioxamine, a hypoxia mimic agent, displayed a much greater increase of these intensified foci in a time-dependent manner. Furthermore, results also show strikingly colocalized foci of γ-H2AX and 53BP1, the latter of which is known to interact with various DNA repair proteins including γ-H2AX in response to DSBs (Schultz et al, 2000). Thus, hypoxic cells experience DNA DSBs, presumably resulting from the NBS1 repression.

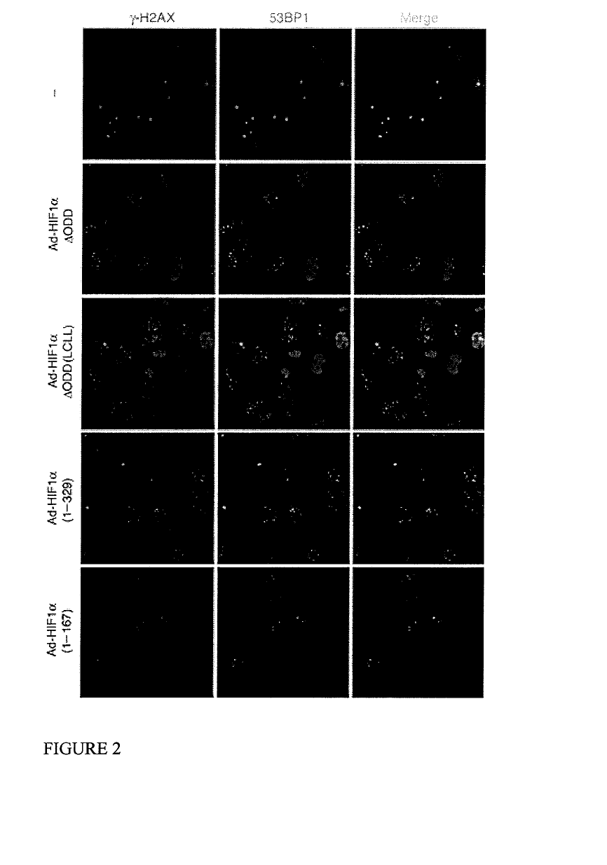

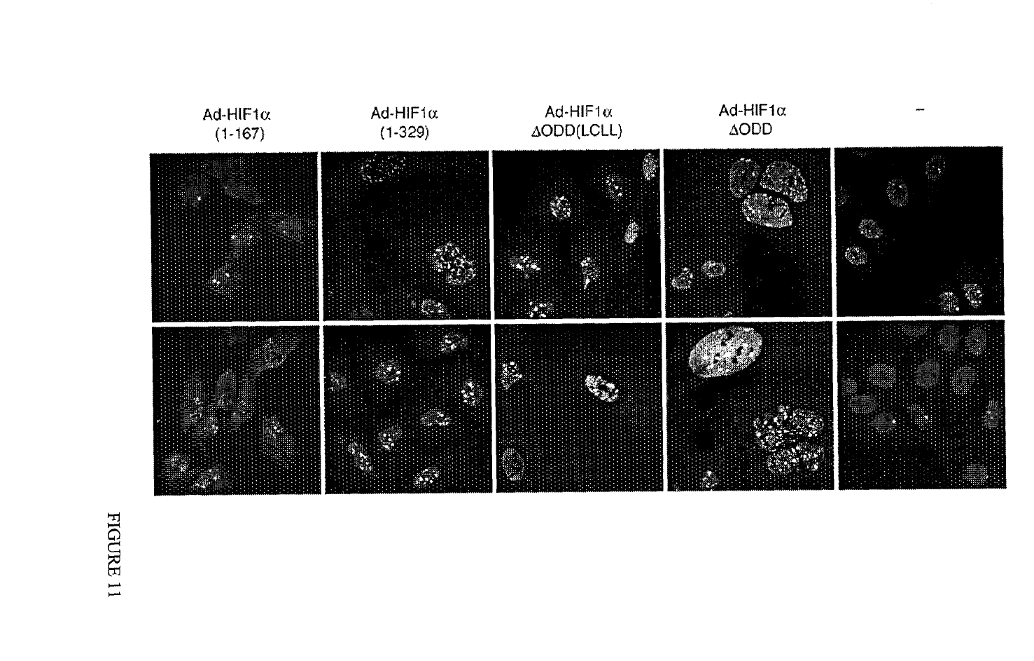

Given its ability to repress NBS1 expression, HIF-1α was tested for the induction of DNA DSBs. Similar to the effects of Ad-HIF1αΔODD and Ad-HIF1αΔODD (LCLL), Ad-HIF-1α (1-329) infection also gave rise to a significant increase in γ-H2AX foci, which were well colocalized with the 53BP1 foci (FIG. 2; FIG. 11; Table 3). Moreover, in accordance with the result above, further removal of PAS-B domain abolished the induction of these foci. Taken together, these results indicate that NBS1 repression by HIF-1α, HIF-1α (1-329) in particular, is associated with increased DNA DSBs.

c) The HIF-1-Myc Pathway is Responsible for NBS1 Repression.

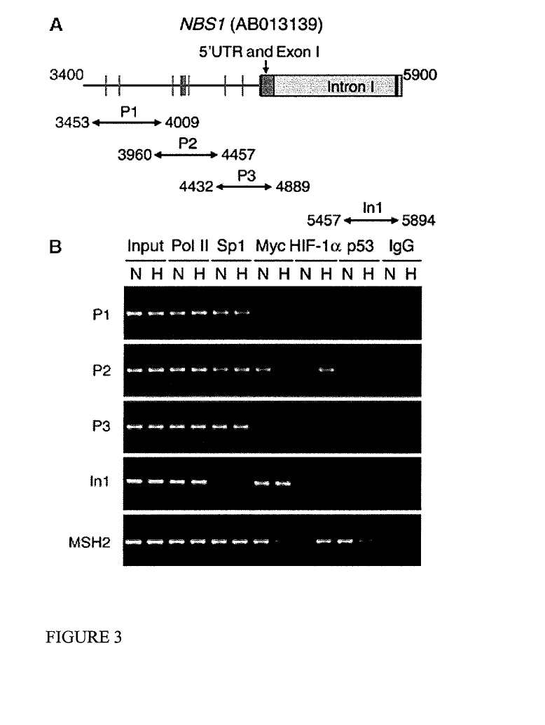

Previously, it was demonstrated that HIF-1 functionally counteracts the transcription factor Myc by altering Myc promoter occupancy (Koshiji et al, 2004, 2005). To test the relevance of Myc displacement in NBS1 repression, chromatin immunoprecipitations were performed to demonstrate the change in occupancy of the NBS1 locus (Matsuura et al, 1998) by the relevant transcription factors under hypoxia. Four sets of PCR primers were used, as illustrated in FIG. 3A, to cover the NBS1 promoter from 1.3 kb upstream of the 5′ untranslated region to 75 base-pair downstream of the ATG start codon, as well as intron 1 harboring an E-box required for Myc-activated transcription (Chiang et al, 2003). Results in FIG. 3B show that in addition to direct binding to the intron, Myc also bound under normoxia to a region clustered with five putative Sp1-binding sites spanned by the primer set P2. In stark contrast to the intron however, the P2 region showed a marked decrease in Myc binding under hypoxia, in concomitance with a gain of HIF-1α binding. It is noteworthy that no Sp1 and HIF-1α binding was detected in intron 1. Furthermore, unlike Myc displacement from the MSH2 promoter requiring wild-type p53 (Koshiji et al, 2005), no p53 binding was present in the scanned regions of NBS1. Thus, it was concluded that HIF-1 selectively displaces Myc from the NBS1 promoter without affecting those bound to the E-box in intron 1.

d) The PAS-B of HIF-1, But Not of HIF-2, is Essential for Sp1 Binding.

Previously, it was shown that Myc displacement is mediated by the N-terminal HIF-1α (1-329), which competes with Myc for Sp1 binding in the MSH2 promoter (Koshiji et al, 2005). To identify the molecular basis that distinguishes HIF-1α from HIF-2α in DNA repair, a series of deletion mutants (FIG. 4A) were constructed to narrow down the region responsible for Sp1 binding. These HIF-1α fragments were translated in vitro in a rabbit reticulocyte lysate system and tested for their binding to the rabbit reticulocyte Sp1. In accordance with a previous finding, deletion of PAS-B resulted in loss of Sp1 binding (FIG. 4B). By contrast, deletion of the bHLH and PAS-A failed to do so, showing that PAS-B (codon 194-329) is sufficient for Sp1 interaction. Of note, removal of part of the PAS-B from the C terminus (codon 299-329) destroyed Sp1 binding.

To provide evidence that HIF-1α PAS-B is sufficient to compete with Myc for Sp1 binding, PAS-B and Myc were translated separately in vitro. As expected, in the absence of one another, Sp1 captured HIF-1 fragments or Myc (FIG. 4C). However, when Myc was mixed with PAS-B or ODD, only the HIF-1α fragments, but not Myc, bound Sp1. Intriguingly, mutation of HIF-1 Thr-327 (T327P, see below) disabled PAS-B. Collectively, these results show that HIF-1α PAS-B is crucial for the HIF-1α-Myc pathway.

HIF-1α PAS-B shares 67% identity in amino-acid sequence with its HIF-2α counterpart, yet functionally HIF-2α differs strikingly in DNA repair. To that end, it was tested whether HIF-2α PAS-B binds Sp1. For simplicity, the HIF-1α PAS-B hereinafter as PAS1B, and the HIF-1α PAS-B (codons 195-331) as PAS2B.

Interestingly, neither full-length HIF-2α nor PAS2B exhibited Sp1-binding activity when expressed in the rabbit reticulocyte lysate (FIG. 4D). Likewise, when they were expressed ectopically in HeLa cells, no Sp1 binding was detected (FIG. 4E). Moreover, neither PAS2B nor the PAS1B T327P mutant was able to compete with PAS1B for Sp1 binding (FIG. 4F). Furthermore, addition of a synthetic peptide corresponding to HIF-1α residues 299-329 (required for Sp1 binding (FIG. 4A)), but not of its HIF-2α counterpart, prevented Sp1 binding in a dose-dependent manner. Thus, it was speculated that the functional divergence between HIF-1α and HIF-2α arises from limited differences in amino-acid sequence spanning from HIF-1α residues 299-329.

e) HIF-2α Pro-329 is Responsible for Abrogating Sp1 Binding.

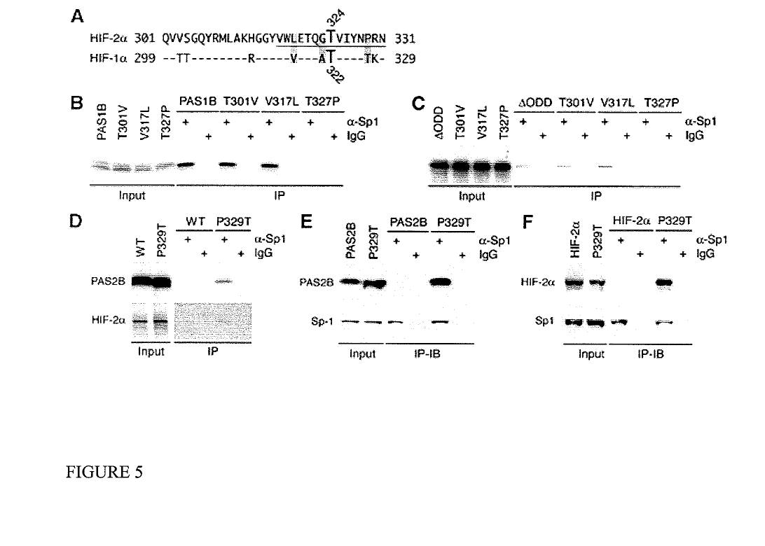

Sequence analysis of HIF-1α residues 299-329 revealed that HIF-1α harbors two unique residues: Thr-301 and Thr-327, corresponding to Val-303 and Pro-329 in HIF-2 (FIG. 5A). Hence, these two residues were replaced with those of HIF-2α. Interestingly, only the PAS1B (T327P) mutant lost Sp1 binding when expressed in the in vitro translation system (FIG. 5B). Similar results were obtained when these substitutions were made in the context of HIF-1αΔODD (FIG. 5C).

Conversely, HIF-2α Pro-329 was substituted with threonine in the context of PAS2B and full-length HIF-2α. Results in FIG. 5D-F show that in contrast to their original forms, these mutants gained Sp1 binding in both cell-free and cell-culture systems. Therefore, HIF-2α Pro-329 is responsible for abrogating Sp1 binding.

f) Phosphorylation of HIF-2α Thr-324 by PKD1 Controls Sp1 Binding.

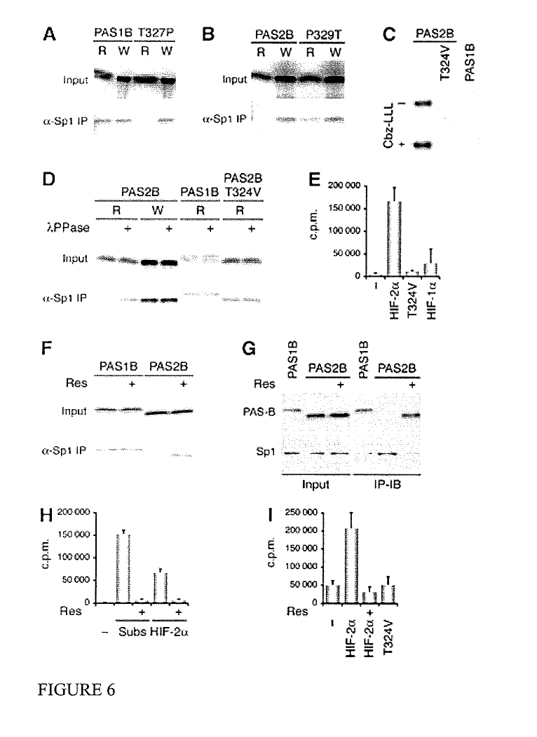

The gain of Sp1 binding by HIF-2 can be a result of substituted threonine, which is presumably subjected to modification required for Sp1 binding. To test this possibility, PAS1B produced in rabbit reticulocyte lysate (modification-proficient) was compared with the one from a wheat germ extract (modification-deficient). Unexpectedly, Sp1 captured PAS produced in both systems (FIG. 6A), showing the irrelevance of threonine modification in Sp1 binding. In keeping with this, the PAS T327P mutant, disabled for Sp1 binding in rabbit reticulocyte, regained binding when produced from the wheat germ, and so did PAS2B (FIG. 6B). These unexpected results indicted (i) no requirement of post-translational modification of HIF-1α for Sp1 binding; (ii) the nonessential role of HIF-1α Thr-327 for the binding; and most importantly (iii) the involvement of post-translational modification of HIF-2α that requires Pro-329 for abrogating Sp1 binding.

To that end, the PAS2B sequence from codon 301-331 (FIG. 5A) was analyzed by a motif-based searching algorithm (http://scansite.mitedu/motifscan_seq.phtml) (Yaffe et al, 2001). Result from a high-stringency search suggested that HIF-2 Thr-324 is harbored within a phosphorylation motif for protein kinase D1 (PKD1), originally classified within the PKC family as PKC (Rykx et al, 2003). Although HIF-1α possesses an equivalent threonine (Thr-322), the algorithm indicates that the lack of a proline at Thr-327 plus the variation of Val-317 and Ala-321 precludes PKD1-mediated phosphorylation (Table 4). In essence, HIF-2α Pro-329 is obligatory for phosphorylation at Thr-324, in support of its role in abrogating Sp1 binding.

To ascertain phosphorylation of HIF-2 Thr-324, in vivo 32P-orthophosphate labeling of ectopically expressed PAS1B and PAS2B. Results in FIG. 6C show that PAS2B, but not PAS1B, was phosphorylated, and furthermore mutation of Thr-324 eliminated the phosphorylation. To demonstrate the effect of phosphorylation on Sp1 binding, PAS2B produced in the rabbit reticulocyte was treated with protein phosphatase.

Dephosphorylation rendered PAS2B able to bind Sp1 (FIG. 6D). Furthermore, mutation of PAS2B Thr-324 to valine also established binding, arguing that dephosphorylation of HIF-2 Thr-324 is required for Sp1 binding. Hence, HIF-2 Thr-324 is phosphorylated in vivo, which functionally distinguishes PAS2B from PAS1B in Sp1 binding.

To determine that it is PKD1 that phosphorylates HIF-2α, recombinant PKD1 was utilized in an in vitro kinase assay. FIG. 6E shows that a HIF-2α peptide, but not its HIF-1α counterpart, was robustly phosphorylated, whereas replacement of Thr-324 with valine abolished phosphorylation. Moreover, incubation with 100-μM resveratrol, a PKD1 inhibitor, during the synthesis of PAS2B in the rabbit reticulocyte established Sp1 binding (FIG. 6F). Likewise, treatment of cells with resveratrol yielded PAS2B-Sp1 immunoprecipitated complexes (FIG. 6G). Furthermore, resveratrol abrogated PKD1-mediated phosphorylation of its consensus substrate peptide as well as the HIF-2α peptide (FIG. 6H), confirming the inhibitory effect of resveratrol on PKD1 kinase activity. Finally, endogenous PKD1 immunoprecipitated from 786-O cells specifically phosphorylated HIF-2α at Thr-324 (FIG. 6I). In aggregate, these results argue that PKD1 phosphorylates HIF-2α Thr-324, thereby precluding Sp1 binding.

g) Nonphosphorylated PAS-B Represses NBS1 Expression and Induces DNA DSBs.

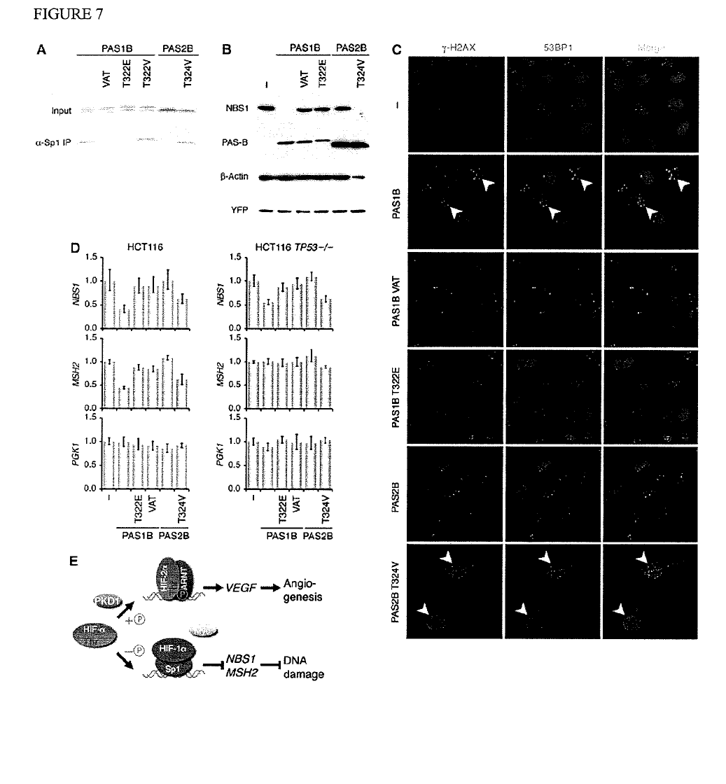

To determine the effect of PAS-B phosphorylation on NBS1 repression, two mutants in PAS1B were created that mimic phosphorylation at Thr-322: one with glutamic acid substitution (T322E), and the other (VAT) undergoing simultaneous replacement of Val-317, Ala-321, and Thr-327 with the corresponding HIF-2α residues (FIG. 5A), yielding an identical score for PKD1 phosphorylation as HIF-2α (Table 4). As expected, both T322E and VAT mutants lost Sp1 binding (FIG. 7A). In contrast, a T322V mutation maintained PAS1B activity, indicating the nonessential role of Thr-322 per se for Sp1 binding. These findings further argue that maintaining HIF-1α Thr-322 or HIF-2 Thr-324 in a nonphosphorylated state is obligatory for Sp1 binding.

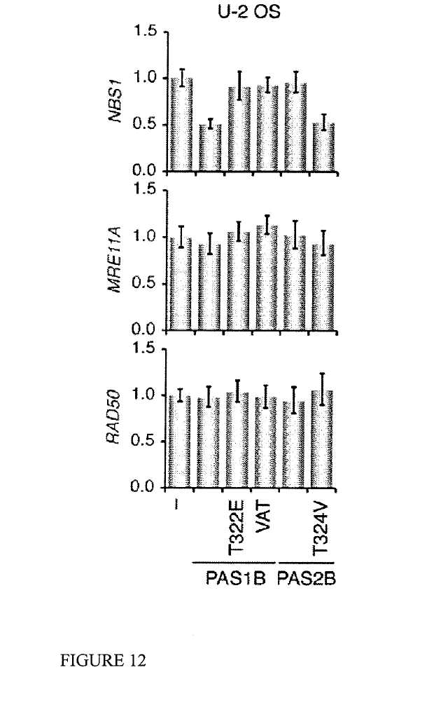

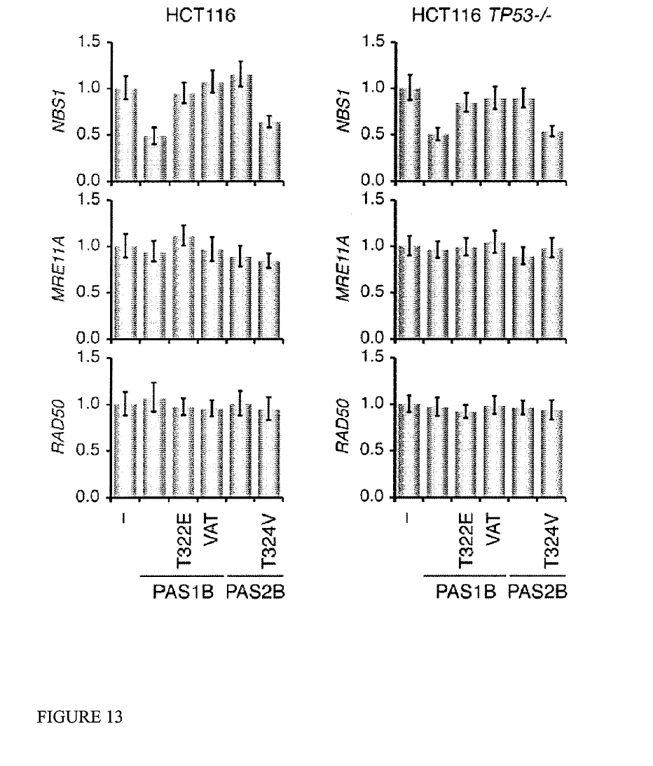

Next, it was asked how these PAS-B mutants relate to their abilities in NBS1 repression. FIG. 7B shows that forced expression of PAS in U-2 OS cells was sufficient to eliminate NBS1 protein levels, whereas the PAS1B VAT and T322E mutants failed to do so. Conversely, the PAS2B T324V mutant, unlike PAS2B, gained its ability to inhibit NBS1 protein levels. Similar effects were observed at NBS1 mRNA levels with these PAS-B variants in both U-2 OS (FIG. 12) and HCT116 cells (FIG. 7D). Again, neither MRE11A nor RAD50 was affected (FIGS. 12 and 13). Interestingly, in TP53−/− HCT116 cells, although MSH2 expression was unaffected by PAS1B and PAS2B T324V, NBS1 mRNA levels were downregulated. This distinct difference is consistent with a p53-independent mechanism for NBS1 repression. Hence, in accordance with the requirement for Sp1 binding, these results underscore the fundamental difference between HIF-1α and HIF-2α in NBS1 repression, resulting from a unique threonine modification in the PAS-B.

To substantiate the role of threonine phosphorylation controlling NBS1 repression, it was asked whether these phosphorylation mutants induce the formation of -H2AX foci. The results in FIG. 7C show that the PAS-B fragments capable to repress NBS1 expression (PAS1B and PAS2B T324V mutant) significantly increased γ-H2AX foci that were colocalized with those of 53BP1. In contrast, those unable to affect NBS1 expression failed to do so. Taken together, these results show that threonine phosphorylation in PAS-B is the molecular determinant that distinguishes HIF-1 from HIF-2 in DNA repair gene regulation.

h) Discussion

Despite its similarities to HIF-1α in amino-acid sequence and protein structure, HIF-2α apparently has separate functions, presumably due to its distinct spatiotemporal expression pattern in vivo (Tian et al, 1998; Compernolle et al, 2002; Scortegagna et al, 2003). However, other studies indicate widespread expression of HIF-2α, along with HIF-1α, in various cell types (Wiesener et al, 1998; Talks et al, 2000). In pursuit of an in-depth understanding of hypoxic effects on DNA repair pathways, it is shown here that HIF-2α, in contrast to HIF-1α, is unable to participate in the HIF-1α-Myc pathway for repressing DNA repair genes, and that this functional difference stems from PKD1-mediated phosphorylation of HIF-2α Thr-324, thereby precluding HIF-2α from competing with Myc for Sp1 binding. The findings show PAS-B phosphorylation serves as a molecular determinant that governs the ability of HIF-α to impair DNA repair and distinguishes between HIF-1α and HIF-2α functionally (FIG. 7E). Of note, PAS-B defined in this study lacks the last β-strand of the structural domain (Erbel et al, 2003), implying a nonessential role for a folded PAS-B in the HIF-1α-Myc pathway. Furthermore, it was observed the induction of apoptosis by HIF-2α overexpression, as reported recently (Acker et al, 2005), along with increased γ-H2AX foci (Rogakou et al, 2000). It is evident that no NBS1 downregulation is involved. Finally, the results show that the NBS1 inhibition by hypoxia and HIF-1α was apparently more effective at protein levels than at mRNA levels, and therefore alternative post-transcriptional mechanisms may also regulate NBS1 expression.

The identification of the PKD1-HIF-2α signaling pathway raises several unanswered questions. Although HIF-1α Thr-322 is not phosphorylated by PKD1, whether other kinases modify this threonine cannot be excluded thus far. Conversely, how HIF-2α phosphorylation is regulated warrants further investigation. PKD1 can be activated thorough PKC-dependent and -independent pathways by various agents including phorbol esters, oxidative stress, tumor necrosis factor α, and ATP, and has been associated with cell proliferation and survival (Rykx et al, 2003). It is particularly interesting to note that HIF-1α plays an essential role in maintaining intracellular ATP levels by stimulating glycolysis and curtailing ATP consumption (To et al, 2005). Furthermore, HIF-1α represses mitochondrial function and O2 consumption by inducing pyruvate dehydrogenase kinase 1 (Kim et al, 2006; Papandreou et al, 2006). Therefore, the maintenance of ATP concentration by HIF-1α might be a contributory factor for maintaining PKD1 activity and thereby HIF-2α phosphorylation. Consequently, HIF-2α tends to function in the canonical hypoxia-responsive pathway for cell proliferation and survival (FIG. 7E).

It is shown here that HIF-1α, the PAS-B in particular, induces DNA DSBs, at least in part, by repressing NBS1 expression. Although previous studies by relying the less-sensitive comet assay indicated no DNA damage under hypoxia (Hammond et al, 2002, 2003), isolated hypoxic S-phase cells exhibited DNA damage when ATR activity was inactivated (Hammond et al, 2004), suggesting the existence of cellular mechanism in response to hypoxia-induced DNA damage. In keeping with this view, apoptosis-defective cells manifested salient genomic alterations including aberrant metaphases and polyploidy changes under hypoxia (Nelson et al, 2004), implicating that apoptosis is part of the hypoxic response to eliminate cells suffering an intolerable amount of DNA damage. Interestingly, the DNA damage response (e.g. by ionizing radiation) in mammalian cells comprises cell-cycle checkpoint activation, transcriptional response, and apoptosis, in addition to DNA repair to maintain genomic stability (Zhou and Elledge, 2000). HIF-1α induces cell-cycle arrest (Carmeliet et al, 1998; Goda et al, 2003; Koshiji et al, 2004; Mack et al, 2005). Given the involvement of NBS1 in cell-cycle checkpoints (D'Amours and Jackson, 2002), it is also interesting to ascertain whether the hypoxic effect on cell cycle is related to the DNA damage arising from hypoxic stress. To that end, the hypoxic response particularly during tumor development comprises activation of cell-cycle checkpoints, transcriptional activation of hypoxia-responsive genes, induction/avoidance of apoptosis, and impairment of DNA repair pathways in order to acquire genetic alterations necessary for tumor survival and progression (To et al, 2005).

In the name of cell survival, the hypoxic response presumably provides opportunities for genetic change. Based on the observation that wild-type p53 is required for the hypoxic impairment of mismatch repair pathway, it was found that hypoxic impairment of mismatch repair occurs during incipient tumorigenesis because majority of the developed cancers harbor mutated p53 (Koshiji et al, 2005). The current study however indicates that hypoxia also participates in tumor progression by inducing chromosomal instability. Although hypoxia-induced DNA DSBs can occur irrespective of the p53 status, whether cells can tolerate the resulting chromosomal aberrations largely depend on the biological integrity of the cells. Cells defective in p53 and apoptosis have a greater propensity to acquire genomic instability during tumor progression (Nelson et al, 2004).

i) Materials and Methods

(1) Plasmids

A series of FLAG-tagged expression plasmids encoding various N-terminal fragments of HIF-1α and HIF-2α were constructed by PCR amplification. The PCR fragments were inserted in-frame into NotI- and XbaI-digested p3XFLAG-CMV10 (Sigma), and were subcloned into BamH1 and XbaI sites in pcDNA3 (Invitrogen). Site-directed mutagenesis was performed as described previously (Huang et al, 2002).

(2) Immunofluorescence

Immunofluorescence staining was performed essentially as described previously (Rogakou et al, 1999) with mouse anti-γ-H2AX antibody (Upstate) and rabbit anti-53BP1 (Novus Biologicals). Secondary antibodies were anti-mouse Alexa-546 and anti-rabbit Alexa-488 (Molecular Probes). Samples were mounted in antifade media, and were examined by fluorescent and laser-scanning confocal microscopy with a Nikon PCM 2000.

(3) In Vivo 32P-orthophosphate Labeling

U2OS cells were transfected with FLAG-tagged PAS1B, PAS2B, or PAS2B T324V mutant. At 48 h after transfection, cells were washed in phosphate-free DMEM, followed by incubation in phosphate-free DMEM for 10 min. Cells were then incubated at 37° C. for 20 min in phosphate-free DMEM containing 100 μCi ³²Pi (phosphorus-32 as orthophosphate in aqueous solution (HCl-free, carrier-free; 10 mCi/ml; Amersham). Subsequently, cells were washed three times with PBS and subjected to anti-FLAG immunoprecipitation. Ectopically expressed PASB was resolved by SDS-PAGE and subjected to autoradiography.

(4) In Vitro Kinase Assay

The procedure for the in vitro kinase assay was modified from a previous report (Storz et al, 2003). Briefly, recombinant PKD1 (Upstate Biotechnology) was used as the source of enzyme in 20 μl of kinase buffer (50 mM Tris/HCl, pH 7.4, 10 mM MgCl₂, 2 mM dithiothreitol). The kinase reaction was carried out for 30 min at room temperature after addition of 10 μl of kinase substrate mixture (150 μM substrate peptide, 50 μM ATP, 10 μCi of [γ-³²P]ATP in kinase buffer). A PKD1-specific substrate (Santa Cruz Biotechnology) was employed as a positive control. A peptide corresponding to HIF-1 residues 299-329 (Ac-GQVTTGQYRMLAKRGGYVWVETQATVIYNTK N-NH2, SEQ ID NO: 3), one including HIF-2α residues 314-331 (Ac-YGRKKRRQRRRGGGVWLETQGTVIYNPRN L-NH2, SEQ ID NO: 4), and another harboring HIF-2α T324V mutation (Ac-YGRKKRRQRRRGGGVWLETQGVVIYNPRN L-NH2, SEQ ID NO: 5) were synthesized by Quality Control Biochemicals. The reactions were terminated by adding 100 μl of 0.75% H3PO4, and 30 μl of the mixed supernatant was spotted to P-81 phosphocellulose paper (Whatman) in triplicates. Papers were washed thoroughly three times with 0.75% phosphoric acid, once with acetone, dried, and radioactivity incorporated into the synthetic peptide was determined in a scintillation counter. Endogenous PKD1 was immunoprecipitated with an anti-PKD1 antibody (SC-935; Santa Cruz) from 786-O cells lysed in a buffer containing 137 mM NaCl, 20 mM Tris (pH 7.5), 1 mM EGTA, 1 mM EDTA, 10% (v/v) glycerol, 1% (v/v) Nonidet P-40, 1 mM vanadate, and protease inhibitor cocktail (Roche). The immune complexes were washed three times with TBS (50 mM Tris/HCl, pH 7.4, 150 mM NaCl) and subjected to in vitro kinase assays.

(5) Cell Culture and Treatment

HCT116 TP53^{+/+}, HCT116 TP53^{-/-} cells (Koshiji et al., 2004), and U-2 OS were grown in McCoy's 5A medium (Invitrogen), and HeLa cells in DMEM. For hypoxic treatment, cells were incubated in a hypoxic chamber (Innova CO-48, New Brunswick Scientific) maintaining 1% O₂ and 5% CO₂. Recombinant adenovirus generation and infection were as previously described (Koshiji et al., 2004).

(6) Real-Time PCR

Total RNA extraction, reverse transcription, and quantitative PCR were performed essentially as described previously (Koshiji et al., 2005). Taqman Gene Expression Assay kits for NBS1, MRE11A, RAD50, MSH2, and ACTB were purchased from Applied Biosystems. All experiments were repeated three times in triplicates, and representative results were presented as means±standard errors.

The PGK1 probe and primers were designed using ABI Primer Express version 2.0 software: probe, 5′-FAM5-ATTTATCTAATTGTCCCATCTCTCCACTGCTGCT-MGBNFQ-3′ (SEQ ID NO: 6); forward primer, 5′-TCTTGAGGAACGGATCAGATGTC-3′(SEQ ID NO: 7); reverse primer, 5′-AGTAGGCCC TTGATAAAGAATGGA-3′ (SEQ ID NO: 8).

(7) RNA Interference

Small interfering RNA for HIF1A and EPAS1 was described previously (Koshiji et al., 2005). The target sequence for HIF1A is 5′-AACTGATGACCAGCAACTTGA-3′ (SEQ ID NO: 9), and the one for EPAS1 is 5′-AACAGCATCTTTGATAGCAGT-3″ (SEQ ID NO: 10). The efficacy of siRNA in each experiment was ascertained by Western blot.

(8) Chromatin Immunoprecipitation

The EZ ChIP™ Chromatin Immunoprecipitation Assay Kit (Upstate) was used for chromatin immunoprecipitations. Briefly, after 16-h treatment with hypoxia (1% O₂), U-2 OS cells were cross-linked with 1% formaldehyde in culture medium at 37° C. for 10 min, and then stopped by adding 125 mM glycine. Cells were lysed in SDS-lysis buffer containing protease inhibitors (2×10⁶ cells/100 μl), and sonicated for eight 10-s pulses (Masonic XL2000; output 5, 20% of maximum power) to produce DNA fragments with an average length of about 200-1000 base-pairs, as determined empirically by agarose gel electrophoresis. The pre-cleared lysate with protein G agarose beads was incubated overnight at 4° C. with specific antibodies, including rabbit anti-Myc, mouse anti-RNA polymerase II (Upstate), mouse anti-p53 (BD Biosciences), mouse anti-Sp1, rabbit anti-HIF-1α, and normal rabbit IgG (Santa Cruz).

PCR analysis was performed in a linear range as determined empirically by agarose gel electrophoresis. The immunoprecipitated DNA fragments were amplified with 30 cycles at 94° C. for 45 s, 55° C. for 1 min, and 72° C. for 1 min. Primer sequences are P1, forward 5′-GCAGAGAGGTTTTTATCCTAAATGGGTG-3′ (SEQ ID NO: 11) and reverse 5′-CAGCACCATGGCTCGCTCCTTTAAT-3′ (SEQ ID NO: 12); P2, forward 5′-CATCTTGGCCTCCCAGACTGCTGG-3′ (SEQ ID NO: 13) and reverse 5′-CCAGTTATGTAGTTTCGTGCGTTTGC-3′ (SEQ ID NO: 14); P3, 5′-GCAAACGCACGAAACTACATAACTGG-3′ (SEQ ID NO: 15) and reverse 5′-TACCGGGAAAATAGGCCCCGAGGCTT-3′ (SEQ ID NO: 16); and In1, forward 5′-ATTGGCAAAGATCTATGTAGAG-3′ (SEQ ID NO: 17) and reverse 5′-TGCCCTACCAGATGGCAAAAT-3′ (SEQ ID NO: 18). The primer sequence for the MSH2 promoter was described previously (Koshiji et al., 2005).

(9) Immunoprecipitation and Western Blot

Immunoprecipitations and Western blot was performed essentially as described previously (Huang et al., 1998; Koshiji et al., 2005). In vitro translation and immunoprecipitations are described as before (Gu et al., 2001; Koshiji et al., 2005). The wheat germ extract system was purchased from Promega. λ protein phosphatase (New England Biolabs) and resveratrol (Sigma) with indicated concentrations were added to the in vitro translated products before immunoprecipitations.

(10) Nucleofection

U-2 OS cells were transfected with the Nucleofector system (Amaxa). Briefly, 1×10⁶ cells were suspended in 100 μl pre-warmed Nucleofector Solution Kit V containing 2 μg plasmids. After electroporation the cells were transferred immediately into pre-warmed complete McCoy's 5A medium and seeded in 6-well plates. Cells were harvested 48 h after transfection.

### 2. Example 2

### HIF-1α PAS-B is Sufficient to Drive Tumor Progression In Vitro

Tumor cells with malignant properties display increased motility and invasiveness and acquired anchorage-independent growth. Therefore, it was expected that these cells expressing HIF-1α PAS-B, resulting from the induction of genetic instability, can exhibit increased mobility in in vitro denudation injury assays and trans-well filter assays. More importantly, these infected cells are expected to be more invasive in Matrigel and to grow on soft agar. In fact, the data has shown that HCT116 cells expressing HIF-1α PAS-B were much more invasive than controls (FIG. 13A). Furthermore, U-2 OS cells, which do not grow on soft agar, formed numerous colonies when HIF-1α PAS was expressed (FIG. 13B). HCT116 is known to form colonies on soft agar (as shown), and it was expected that HIF-1α PAS-B expressed HCT116 can grow more and larger colonies at a given time point in reference to the parental cells. The controlled cells, especially those expressing the HIF-1α PAS-B mutant, can behave like the parental cells. The results can strongly indicate a critical role of HIF-1α PAS-B in tumor progression in vitro and the involvement of the HIF-1α-Myc pathway that is known to induce genetic instability.

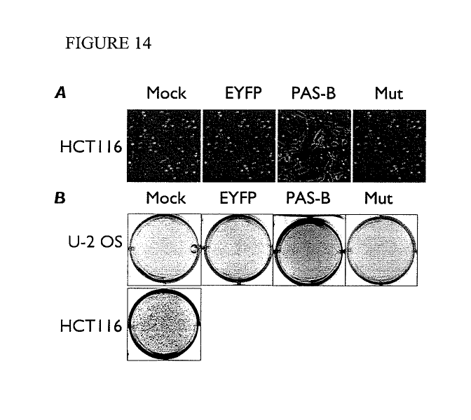

FIG. 14 shows HIF-1α PAS-B expression promotes malignant properties in HCT116 and U-2 OS cells. A, retrovirally infected HCT116 cells expressing EYFP, HIF-1α PAS-B, and mutant were assayed for Matrigel invasion. Images of cells on the membrane side were taken 24 h later. B, U-2 OS cells infected as above were assayed for anchorage-independent growth on soft agar. HIF-1α PAS-B expressed cells gave rise to formation of colonies 40 times more than others. HCT116 cells (known to grow on soft agar) were used as a positive control. Mock, uninfected cells.

HIF-1α PAS-B expression induces epithelial-messenchymal transition in colon and osteosarcoma cell lines

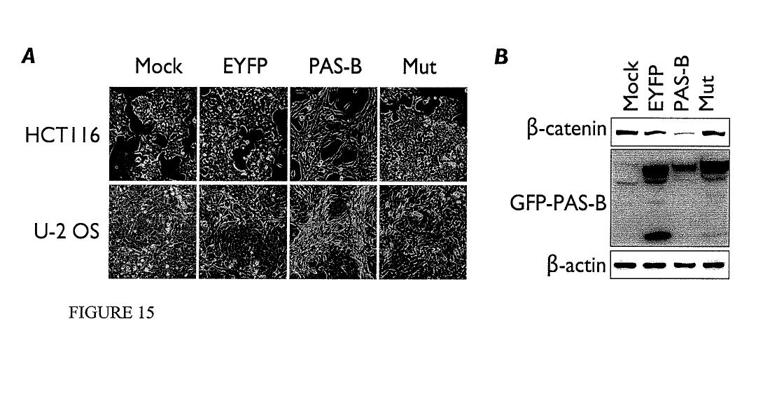

Previously, it was demonstrated that HIF-1α utilizes its PAS-B to induce genetic instability by inhibiting DNA repair gene expression (Koshiji et al., 2005; To et al., 2006). Recent data show that HIF-1α PAS-B expression promoted tumor cell invasion and anchorage-independent growth. Moreover, a striking phenotypic change from an epithelium-like morphology to fibroblast-like after the introduction of HIF-1α PAS-B in both HCT116 and U-2 OS has been shown (FIG. 15).

FIG. 15 shows HIF-1α PAS-B expression in HCT116 and U-2 OS cells results in striking phenotypic changes. A, Cells as indicated were infected by retroviruses expressing EYFP, EYFP-PAS-B fusion, and EYFP-PAS-B mutant. Both HIF-1α PAS-B expressed HCT116 and U-2 OS cells exhibit fibroblast-like morphology. B, U-2 OS and those infected with retroviruses as above were analyzed by immunoblotting individual protein expression as indicated. There is a marked decrease in the epithelial marker β-catenin in cells expressing HIF-1α PAS-B.

HIF-1α PAS-B expression accelerates tumor formation in vivo

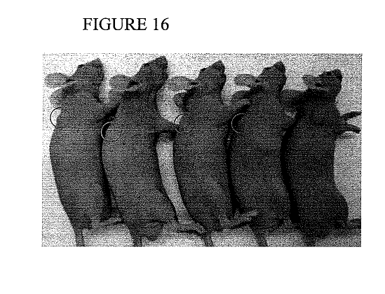

To provide evidence that the HIF-1α-Myc pathway drives tumor progression in vivo, tumor xenograft mouse models can be used. In fact, in the initial trial where subcutaneous injections of U-2 OS cells were performed in ten athymic mice, only cells that expressed HIF-1α PAS-B but not the mutant formed tumor nodules within three weeks (FIG. 16).

FIG. 16 shows HIF-1α PAS-B expression accelerates tumor formation in a xenografted mouse model. Female BALB/c-nu/nu mice were subjected to bilateral, subcutaneous injections in the back with 1 million of U-2 OS cells, or those expressing EYFP, HIF-1α PAS-B, or the HIF-1α PAS-B mutant. A total of ten mice were divided into two groups with one type of cells injected on one side and another on the other side.

Three weeks after, only those injected with HIF-1α PAS-B expressed cells developed tumor nodules (as circled).

### 3. Example 3

### Hypoxic Suppression of the Cell-Cycle Gene CDC25A in Tumor Cells

Hypoxia, a key microenvironmental factor for tumor development, not only stimulates angiogenesis and glycolysis for tumor expansion, but also induces cell-cycle arrest and genetic instability for tumor progression. Several independent studies have shown hypoxic blockade of cell-cycle progression at the G₁-S transition, arising from the inactivation of S-phase-promoting cyclin E-CDK2 kinase complex. Despite these findings, the biochemical pathways leading to the cell-cycle arrest previously remained poorly defined. It was recently shown that hypoxia activates the expression of CDNK1A encoding the CDK2 inhibitor p21^{Cip1} through a novel HIF-1α-Myc pathway that involves Myc displacement from the CDNK1A promoter by the hypoxia-inducible transcription factor HIF-1α. In pursuit of further understanding of the hypoxic effects on cell cycle in tumor cells, it is herein shown that hypoxia inhibits the expression of CDC25A, another cell-cycle gene encoding a tyrosine phosphatase that maintains CDK2 activity. In accordance with the HIF-1α-Myc pathway, hypoxia requires HIF-1α for CDC25A repression, resulting in a selective displacement of an activating Myc from the CDC25A promoter that lacks a canonical E-box without affecting Myc binding in the intron. These studies show that hypoxia inhibits cell-cycle progression by controlling the expression of various cell-cycle genes.

Cellular hypoxia is an environmental stress with important implications in developmental biology, normal physiology and many pathological conditions, including cancer (Bunn et al. 1996; Semenza 1999; Wenger 2002). Cells adapt to hypoxia in various ways, such as metabolic transition to glycolysis from oxidative phosphorylation, vessel dilation, and neovascularization. Many of these cellular responses are mediated by HIF-1α, a basic helix-loop-helix transcription factor that is constitutively transcribed and translated in normoxia, but rapidly degraded by the ubiquitin-proteasome pathway (Huang et al. 2003). Hypoxia inhibits HIF-1α ubiquitinylation (Pugh et al. 2003; Poellinger et al. 2004; Kaelin 2005), resulting in HIF-1α stabilization, dimerization with ARNT, and recruitment of transcriptional co-activators (Huang et al. 2003). Consequently, the HIF-1α-ARNT heterodimer binds to hypoxia-responsive elements in the target gene promoter, thereby activating transcription of genes encoding vascular endothelial growth factor, glucose transporters, and glycolytic enzymes for cell growth and survival (Semenza 1999).

In addition to the induction of angiogenesis and glycolysis, hypoxia also inhibits cell-cycle progression by blocking the G₁-S transition, even though the underlying mechanisms may vary according to the experimental conditions (Carmeliet et al. 1998; Gardner et al. 2001; Goda et al. 2003; Koshiji et al. 2004a). It has been shown that hypoxic inhibition of cell-cycle progression requires the up-regulation of the cyclin-dependent kinase inhibitor genes CDKN1A (encoding p21^{Cip1}) and/or CDKN1B (encoding p27^{Kip1}) (Carmeliet et al. 1998; Gardner et al. 2001; Goda et al. 2003), resulting in the inactivation of the S-phase-promoting cyclin E-CDK2 kinase complex. In addition, hypoxia induces hypophosphorylation of the retinoblastoma protein (Gardner et al. 2001; Goda et al. 2003), thereby inhibiting the E2F-dependent transcription of S-phase genes.

Interestingly, the activation of cyclin E-CDK2 complex also requires CDC25A, a tyrosine phosphatase that removes the inhibitory phosphorylation of CDK2 Tyr-15 (Gu et al. 1992; Jinno et al. 1994). Therefore, changes in CDC25A gene expression alter CDK2 activity. CDC25A is transcriptionally activated by the transcription factors Myc (Galaktionov et al. 1996) and E2F after serum stimulation (Vigo et al. 1999; Chen 1999), but repressed by the recruitment of retinoblastoma protein family members and histone deacetylase to the E2F DNA binding site following TGF-β treatment (Ivarone and Massague 1999). Alternatively, upon DNA damage the CDC25A phosphatase turns labile, resulting from ATM-Chk2/ATR-Chk1 mediated phosphorylation that targets CDC25A for proteolysis (Mailand et al. 2000). Likewise, interleukin-7 induces CDC25A degradation in T lymphocytes upon phosphorylation by p38 MAPK (Kittipatarin et al. 2000) These findings have led to the proposal of a “two-wave” concept for the G₁ checkpoint in response to the genotoxic stress: the CDC25A proteolysis constitutes a rapid inhibition of the cyclin E-CDK2 complex for G₁ arrest, while a sustained cell-cycle arrest requires transcriptional up-regulation of the CDK2 inhibitors (Bartek et al. 2001).

Recently a novel mechanism of HIF-1α action (referred to as the HIF-1α-Myc pathway) was identified that accounts for CDKN1A up-regulation by hypoxia (Koshiji et al. 2004a; Koshiji et al. 2004b). In contrast to the canonical activation pathway by which HIF-1α activates hypoxia-responsive genes via binding to the hypoxia-responsive element, in the HIF-1α-Myc pathway HIF-1α functionally counteracts the repressive activity of Myc by displacing Myc from the CDKN1A promoter via protein-protein interactions. As such, neither HIF-1α DNA-binding domain nor its transcriptional activation domain is required for the CDKN1A up-regulation. Moreover, this HIF-1α-Myc pathway is also applicable to the hypoxic down-regulation of DNA repair genes MSH2 and NBS1(Koshiji et al. 2005; To et al. 2006), which are activated by Myc. Thus, this mechanism accounts for a new group of hypoxia-responsive genes that generally lack the hypoxia-responsive element in the promoter and are either up- or down-regulated by hypoxia. In search of additional genes involved in cell-cycle arrest under hypoxia, oligonucleotide microarray analysis was performed in the human colon cancer cell line HCT116, and identified another cell-cycle gene, CDC25A, which was down-regulated by hypoxia.

a) Materials and Methods

Cell Culture and Treatment. HCT116 p53^{+/+}, HCT116 p53^{-/-} and HCT116 p21^{-/-} were cultured in McCoy's 5A medium, MCF7 and HeLa cells in DMEM and Hep3B cells in MEM medium. All the media contained 10% fetal bovine serum (Hyclone), penicillin and streptomycin. Reagents unless otherwise noted were obtained from Sigma. Hypoxic conditions were maintained at 1% oxygen as described previously (Kageyama et al. 2004). Recombinant adenoviruses and adenoviral infections were essentially as described before (Koshiji et al. 2004a).

Oligonucleotide-Microarray Hybridization and Analysis. HCT116 cells were incubated for 16 h under hypoxic or normoxic conditions, and total RNA was extracted with TRIzol reagent (Invitrogen). Microarray analysis was performed according to the protocols available at http://nciarray.nci.nih.gov. In brief, fluorescently labeled cDNA targets were generated using 40 μg of total RNA with a single round of reverse transcription in the presence of aminoallyl-dUTP (Sigma), followed by a coupling reaction to Cy3 or Cy5 monofunctional NHS-ester (Amersham Pharmacia). Complex targets (usually containing normoxic cDNA labeled with Cy3 and hypoxic cDNA with Cy5) were denatured and hybridized to glass slides featuring 22,272 oligonucleotide elements (NCI Microarray Facility) at 42° C. overnight. Slides were washed successively in 1×SSC/0.1% SDS, 1×SSC and 0.2×SSC for 2 min each, and spin-dried. The intensities of Cy3 and Cy5 were determined simultaneously by using a GenePix 4000A scanner, and the acquired images were processed with GenePix Pro 3.0 software (Axon Instruments). The basic raw data and derived ratio measurements were then uploaded to the NCI MicroArray Database System and further processed by using the BRB-ArrayTools software (NCI Biometric Research Branch). Three independent samples were performed for each condition. After filtering out genes with low variability within the whole dataset, the t-test Class Comparison Tool within the BRB-ArrayTools software was used to find genes differentially expressed between the normoxia and the hypoxia group at a significance level of 0.005 based on 2000 random permutations.

Reverse transcription polymerase chain reaction (RT-PCR)—Total RNA was extracted using TRIzol (Invitrogen) according to the manufacturers recommendations. After reverse transcription with PowerScript RT (Clontech), cDNA from 125 ng RNA was amplified with Taq DNA polymerase (Promega) in 30 amplification cycles. Specific primers (synthesized by IDT DNA Technologies) were as follows: HIF-1α, forward 5′-CCGGAATTCTCAACCACAGTGCATTG-3′ (SEQ ID NO: 19), reverse 5′-CGGGATCCATACGGTCTTTTGTCACTG-3′(SEQ ID NO: 20); HIF-2α, forward 5′-AAGCCTTGGAGGGTTTCATT-3′(SEQ ID NO: 21), reverse 5′-TGCTGGATTGGTTCACACAT-3′(SEQ ID NO: 22); β-actin forward 5′-GTGGGGCGCCCCAGGCACCA-3′(SEQ ID NO: 23), reverse 5′-CTCCTTAATGTCACGCACGATTTC-3′(SEQ ID NO: 24). PCR products were resolved on 2% agarose gels stained with ethidium bromide.

Real-Time RT-PCR. Real-time RT-PCR was performed essentially as described previously (Koshiji et al. 2005). Primer and probe (Applied Biosystems) sequences were as follows: CDC25A, forward 5′-CTGGGACTTCCATGCCTTAAAC-3′ (SEQ ID NO: 25), reverse 5′-GCCCTGGGCTCCAACCT-3′ (SEQ ID NO: 26), probe 5′-FAM-ACCTCCCACACTCC-MGB-3′ (SEQ ID NO: 27). PGK-1 (FAM-MGB probe) and ACTB (VIC-MGB probe) were detected with endogenous control reagents from Applied Biosystems. β-actin primers and probe were included into each reaction for normalization. All the experiments were repeated three times in triplicate, and representative results were presented in mean±standard error.

Western Blot. Western blot analysis was carried out as described previously. (Huang et al 1998) Monoclonal CDC25A (F-6) antibody was purchased from Santa Cruz, monoclonal HIF-1α antibody from BD Biosciences, polyclonal HIF-2α antibody from Novus Biologicals, and polyclonal Chk1 antibody from Cell Signaling Technology.

RNA Interference. Transfection with siRNA duplexes and siRNA sequence information were described previously (Koshiji et al. 2005). CHK1 siRNA was described previously (Zhao et al. 2002). Luciferase siRNA was used as negative control.

Reporter Assay. Reporter assays were conducted as described previously (Huang et al. 2002). The Myc effect on the reporter was examined by cotransfection with 0.4 μg pMyc (Koshiji et al. 2004a) or as specified.

Chromatin Immunoprecipitation. Chromatin immunoprecipitations were preformed essentially as described previously (Koshiji et al. 2004a; Koshiji et al. 2005) with indicated antibodies described before. Primer sequences of the natural promoter region (−446 to +59 in reference to the transcription start site of the gene locus AF527417) were forward 5′-CAGACCTCCACAGGTCTTCC-3′ (SEQ ID NO: 28) and reverse 5′-CAGAAAACCAAGCCGACCTA-3′ (SEQ ID NO: 29). Those flanking the Myc binding region within the second intron (+2787 to +3256) were forward 5′-AACTCTGTCACCCAGGCAAC-3′ (SEQ ID NO: 30), and reverse 5′-GCTCACACCTGTGATTCCAA-3′ (SEQ ID NO: 31).

b) Results

Profiling of hypoxia-regulated genes in human colon cancer cells. To gain an insight into the expression profile of hypoxia-regulated genes in human colon cancers, an oligonucleotide microarray analysis of HCT116 cells that had been subjected to normoxic or hypoxic conditions for 16 h was performed. The gene profiling from three independent samples per condition revealed a significant change of 446 genes with a p-value<0.005. Using a 2.5-fold cutoff, 47 genes were shown to be up-regulated, whereas 3 genes down-regulated. Consistent with previous reports (for review see Semenza 2003), majority of the identified genes are involved in metabolic and glycolytic processes, as well as in cell adhesion and angiogenesis. The most interesting finding, however, was that 40% of the genes in the dataset play major roles in cell growth, differentiation and survival (Table 5), some of which are also oppositely regulated by Myc. These include Myc-repressed genes that are up-regulated by hypoxia, such as cyclin G2 (CCNG2), plastin 3 (PLS3), ERBB receptor feedback inhibitor 1 (ERRF11), and N-myc downstream-regulated gene 1 (NDRG1), and the Myc-activated gene CDC25A that is suppressed by hypoxia.

It was recently demonstrated that HIF-1α mediates hypoxia induced cell-cycle arrest in HCT116 cells, resulting from HIF-1α counteraction of Myc that represses CDKN1A transcription (Koshiji et al. 2004a). It appears that the hypoxia-induced CDC25A repression is a result of the same mechanism.

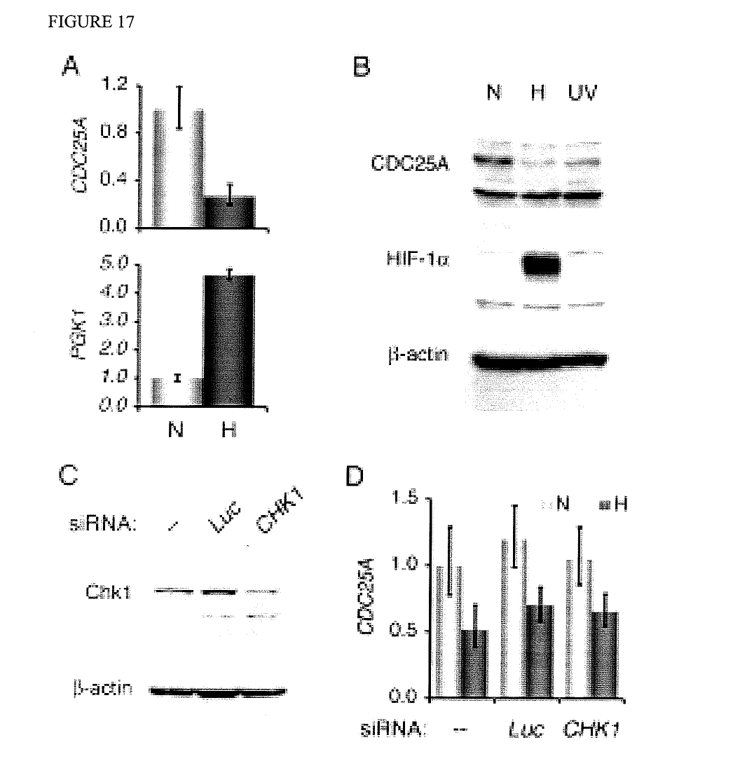

Hypoxia inhibits CDC25A expression in HCT116 cells independent of the ATR-Chk1 pathway. To validate CDC25A down-regulation by hypoxia, real-time PCR analysis in HCT116 cells was performed. As shown in FIG. 17A, CDC25A mRNA levels were reduced by ˜threefold after a 16-h hypoxic treatment, which is well in accordance with the reduction seen in the microarray analysis. As expected, the mRNA levels of hypoxia-inducible glycolytic enzyme gene PGK1 were markedly increased. The inhibition of CDC25A expression occurred 8 h after the hypoxic treatment and persisted throughout a 24-h treatment. Furthermore, hypoxia also substantially reduced CDC25A protein levels (FIG. 17B), which were similar to those arising from CDC25A protein degradation upon ultra-violet irradiation. Therefore, these results confirmed that hypoxia specifically down-regulates CDC25A expression in HCT116 cells.

To explore the potential mechanism, the possible involvement of the ATR-Chk1 pathway was tested that mediates ubiquitin-proteasome degradation of CDC25A by phosphorylation, (Mailand et al. 2000) given the implication of an ATR mediated signaling pathway in hypoxia-induced cell-growth arrest and phosphorylation of p53 and histone H2AX (Hammond et al. 2002; Hammond et al. 2003). To that end, CHK1 gene was targeted by siRNA for gene silencing. Western blot analysis showed ˜80% reduction of the Chk1 protein by siRNA transfection (FIG. 17C). However, hypoxia still inhibited CDC25A mRNA levels (FIG. 17D), showing that CDC25A repression by hypoxia is independent of the ATR-Chk1 pathway.

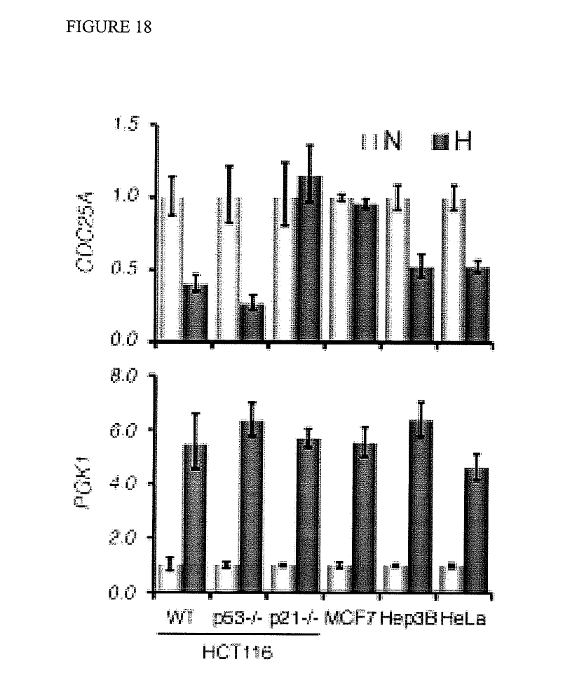

Nonessential role of p53 in CDC25A down-regulation by hypoxia. The tumor suppressor p53 has been shown to play an essential role in cell-cycle arrest upon DNA damage. Although it appears to be dispensable in the hypoxic up-regulation of CDKN1A (Koshiji et al. 2004a; Koshiji et al. 2005), p53 has been shown recently to down-regulate CDC25A expression (Rother et al. 2007). To test the possible involvement of p53 in CDC25A repression by hypoxia, a number of cells with known p53 status were surveyed. As shown in FIG. 18, HCT116 p53^{-/-} (p53-null), Hep3B (p53-mutant) and HeLa (p53-inactivated) cells exhibited a similar reduction of CDC25A mRNA levels by hypoxia. By contrast, two p53 wild-type cell lines, HCT116 p21^{-/-} and MCF7, lacked the hypoxic inhibition. Therefore, these data show that p53 is nonessential for CDC25A repression by hypoxia.

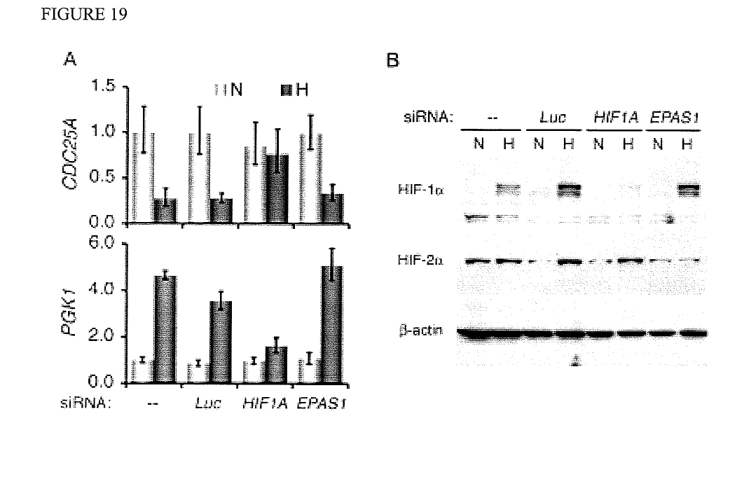

HIF-1α is required for hypoxia-induced CDC25A repression. Next, the requirement of the transcription factor HIF-α for the CDC25A repression was ascertained. HCT116 cells were transfected with siRNA duplexes targeting HIF1A or EPAS1 (encoding HIF-2α, another member of the HIF-α family), and with luciferase siRNA as a control. FIG. 19A shows that HIF1A siRNA not only effectively blocked the CDC25A down-regulation (top panel) but also abolished the PGK1 up-regulation (bottom panel). Likewise, hypoxic inhibition of a CDC25A reporter expression was also abrogated by the use of HIF1A siRNA. In contrast, EPAS1 siRNA had no obvious effects on the expression of CDC25A and PGK1. Similar results were obtained in HCT116 p53^{-/-} cells.

To confirm that HIF1A and EPAS1 expression were inhibited by their respective siRNAs, the target gene expression at their mRNA and protein levels was examined. Results in FIG. 19B show that transfection with HIF1A siRNA resulted in >80% decrease of HIF-1α protein levels, while HIF-2α levels remained unaffected. Likewise, HIF-2α levels were reduced specifically by the EPAS1 siRNA. Therefore, it was concluded that HIF-1α, but not HIF-2α, is involved in hypoxia-induced CDC25A repression.

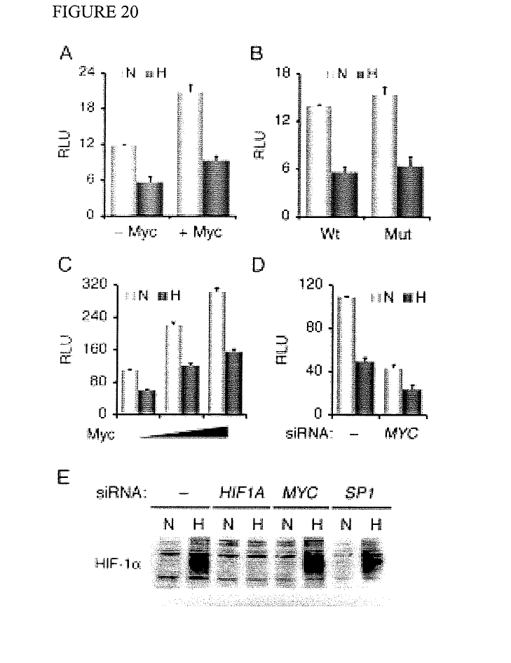

Hypoxia inhibits CDC25A transcription by Myc displacement in the promoter. To investigate whether the hypoxic down-regulation of CDC25A mRNA levels occurs at the transcriptional level, luciferase assays were performed by using initially a CDC25A reporter construct that consists of the natural promoter region as well as the intron 2 harboring a functional Myc-binding E-box. Accordingly, the CDC25A reporter construct was Myc-inducible (FIG. 20A). Although the induction of Myc activity was exhibited in normoxia as well as in hypoxia, a modest decrease of the stimulation was observed under hypoxia. Furthermore, the hypoxic treatment resulted in >50% decrease of reporter activities, irrespective of forced Myc expression. Interestingly, mutation of the Myc-binding E-box failed to abrogate the hypoxic repression (FIG. 20B), showing that hypoxic inhibition of CDC25A transcription is independent of the Myc-binding activity in intron 2. In keeping with this notion, a second CDC25A reporter containing only the promoter region was also stimulated by ectopic Myc expression in a dosage effect (FIG. 20C), even though no canonical Myc-binding E-box has been reported in the CDC25A promoter. These findings suggest that in the absence of intron 2, the CDC25A promoter responds to Myc stimulation, which can be reversed by hypoxia. To confirm the functional role of Myc in CDC25A promoter activation, cells were transfected with MYC siRNA and, as shown in FIG. 20D, there was a 60% decrease of CDC25A promoter activities by the MYC siRNA under normoxia, with an additional 18% decrease under hypoxia. The residual inhibition under hypoxia can be a result of the incomplete knockdown of Myc protein levels that were subjected to further reduction by hypoxia (FIG. 20E).

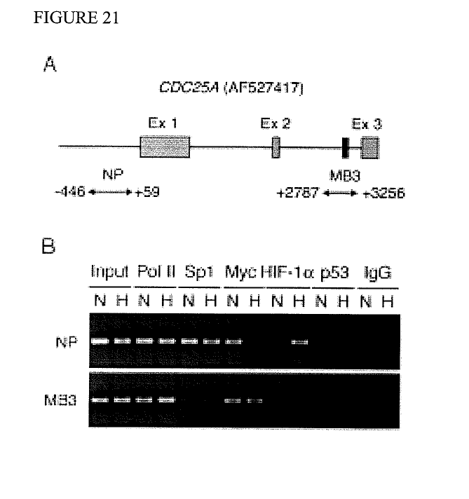

To gain further insight into the mechanism of the transcriptional repression of CDC25A, chromatin immunoprecipitations were performed to ascertain whether the HIF-1α-Myc pathway is applicable to the CDC25A inhibition. Two sets of PCR primers were used (FIG. 21A); one spans the natural promoter region of CDC25A harboring AP-2, Sp1 and E2F binding sites (Chen et al. 1999; Iavarone et al. 1999), and the other the Myc-binding region 3 (MB3) in intron 2 (Galaktionov et al. 1996). Results in FIG. 20B show that in addition to binding to the MB3 under normoxia, Myc also bound to the promoter. Moreover, under hypoxia the promoter lost Myc binding but gained HIF-1α binding. Remarkably, despite the reduction of Myc protein levels by hypoxia (FIG. 20E), Myc binding to MB3 remained unchanged. These results indicate that hypoxia induces Myc displacement in the CDC25A promoter that lacks a canonical E-box, which is in agreement with the results of FIG. 20 and previous findings (Koshiji et al. 2005; To et al. 2006). Taken together, these findings show that hypoxia inhibits CDC25A gene expression by selectively displacing Myc binding in the promoter region. In addition, no p53 binding was detected in the promoter (Rother et al. 2007) and the intron, even though Sp1 interacted only with the promoter. Sp1 has been shown to be a mediator of the HIF-1α-Myc pathway (Koshiji et al. 2005), and accordingly knockdown of Sp1 expression (FIG. 20E) not only markedly repressed the CDC25A promoter activity but also blocked the hypoxic inhibition, further supporting the involvement of the HIF-1α-Myc pathway in CDC25A repression by hypoxia.

c) Discussion

Substantial evidence indicate that hypoxia inhibits cell-cycle progression by blocking G₁-S transition, resulting primarily from the up-regulation of cyclin-dependent kinase inhibitor p21^{Cip1} and/or p27^{Kip18-10} and the hypophosphorylation of the retinoblastoma protein (Gardner et al. 2001; Goda et al. 2003). The identification of the HIF-1α-Myc pathway has provided new insight into the mechanisms of hypoxia-responsive genes lacking the canonical hypoxia-responsive element in the promoter (Koshiji et al. 2004a). Recently, an independent study also showed that this mechanism is relevant to the up-regulation of p21^{Cip1} and p27^{Kip1} in Vhl^{-/-} fibrosarcomas (Mack et al. 2005). While the HIF-1α-Myc pathway was proposed originally to account for the up-regulation of CDKN1A by hypoxia, the current study indicated that HIF-1α is also involved in hypoxic down-regulation of CDC25A. Remarkably, the opposite patterns of gene expression mediated by HIF-1α converge toward the inactivation of S-phase-promoting cyclin E-CDK2 kinase complex, essential for cells entering S phase. Altogether, the findings show that HIF-1α coherently regulates hypoxia-responsive genes critical for cell-cycle arrest.

It is noteworthy that unlike ATR-Chk1 mediated CDC25A proteolysis following DNA damage, which constitutes the immediate response of the “two-wave” cell-cycle arrest concept (Bartek et al. (2001), the hypoxic down-regulation of CDC25A expression presumably is not an immediate action. Moreover, cell-cycle arrest by DNA damage involves p53-independent CDC25A proteolysis and p53-dependent up-regulation of CDKN1A, whereas the hypoxic effects on cell cycle appear to be p53-independent and so are the cell-cycle genes regulated by hypoxia. Therefore, it is less likely that the hypoxic effects on cell cycle conform to the “two-wave” concept of cell-cycle arrest by DNA damage. Apart from its role for G₁ arrest, CDC25A has been shown to be involved in the S and the G₂-M arrest (Bartek et al. 2004). In correlation, hypoxic tumor cells are also accumulated in these phases of the cell cycle.

Previously, it was demonstrated that HIF-1α competes with Myc for Sp1 binding to the MSH2 promoter for gene repression, indicating that it is an indirectly DNA-bound Myc that is displaced by HIF-1α Koshiji et al. 2005; To et al. 2005). In this study, evidence that hypoxia-induced Myc displacement occurs selectively in the CDC25A promoter lacking a canonical E-box rather in the intron harboring a canonical E-box is provided. The identification of functional Myc binding to the CDC25A promoter is consistent with recent reports (Vigneron et al. 2006; Barre et al. 2005).

Although both CDC25A and MSH2 are down-regulated by hypoxia via the HIF-1α-Myc pathway, unlike MSH2 regulation, the CDC25A repression is p53-independent. Likewise, hypoxic activation of CDKN1A is also p53-independent (Goda et al. 2003; Koshiji et al. 2005).

TABLE 3
Quantification of γ-112AX foci per cell
induced by hypoxia and HIF-1α

| Treatment^{a} | Mean | Standard Error |
|---|---|---|
| Normoxia | 2.38 | 0.31 |
| Hypoxia | 6.58 | 0.41 |
| Desferrioxamine | 40.62 | 4.14 |
| Treatment^{b} | Mean | Standard Error |
| Noninfected | 3.71 | .066 |
| Ad-HIFIαΔODD | 19.67 | 1.90 |
| Ad-HIFIαΔODD(LCLL) | 12.71 | 1.04 |
| Ad-HIFIαΔODD (1-329( | 13.52 | 2.26 |
| Ad-HIFIαΔODD (1-167) | 5.55 | .066 |

^{a}U-2 OS cells were maintained under normoxia, or subjected to hypypoxic or desferrioxamine conditions for 72 h and stained by immunofluorescence with antibodies against γ-H2AX and 53BP1. Randomly selected cells were counted for γ-H2AX foci and presented in mean and standard error per cell.
^{b}U-2 OS cells were infected with adenoviruses expressing HIF-1α variants as indicated, and stained by immunofluorescence with antibodies against γ-H2AX and 53BP1. Randomly selected cells were counted for γ-H2AX foci and presented in mean and standard error per cell.

TABLE 4
Predicted phosphorylation sites in HIF-1α and
HIF-2α PAS-B.

| | | Kinase | |
|---|---|---|---|
| | Sequencea | Motif | Score |
| HIF-2α | 317-VWLETQGTVIYNPRN-331 | PKD1 | 0.3720 |
| HIF-1α | 315-VWVETQATVIYNTKN-329 | — | — |
| HIF-1αVAT | 315-VWLETQGTVIYNPKN-329 | PKD1 | 0.3720 |

^{a}The predicted phosphorylation site by high stringency is shown in bold: HIF-1α residues different from those of HIF-2α in italics; and mutated residues are underlined.

TABLE 5
Hypoxia-responsive genes related to cell growth, differentiation and survival

| Fold | UniGene | | Gene |
|---|---|---|---|
| Change | ID | Gene Description | Symbol |
| 11.429 | Hs.523012 | DNA-damage-inducible transcript 4 | DDIT4 |
| 8.567 | Hs.372914 | N-myc downstream regulated gene 1 | NDRG1 |
| 7.739 | Hs.144873 | BCL2/adenovirus E1B 19 kDa interacting protein 3 | BNIP3 |
| 6.746 | Hs.131226 | BCL2/adenovirus E1B 19 kDa interacting protein 3-like | BNIP3L |
| 5.541 | Hs.171825 | basic helix-loop-helix domain containing, class B, 2 | BHLHB2 |
| 5.197 | Hs.53 1819 | jumonji domain containing 1A | JMJD1A |
| 4.947 | Hs.13291 | cyclin G2 | CCNG2 |
| 4.64 1 | Hs.7089 | insulin induced gene 2 | INSIG2 |
| 4.555 | Hs.80342 | keratin 15 | KRT15 |
| 3.923 | Hs.436944 | sprouty homolog 1, antagonist of FGF signaling (Drosophila) | SPRY1 |
| 3.760 | Hs.142395 | growth and transformation-dependent protein | E2IG5 |
| 3.209 | Hs.50 1023 | MAX interactor 1 | MXI1 |
| 3.192 | Hs.220971 | FOS-like antigen 2 | FOSL2 |
| 3.145 | Hs.1 1169 | ERBB receptor feedback inhibitor 1 | ERRFI1 |
| 3.033 | Hs.1 16479 | lysyl oxidase-like 2 | LOXL2 |
| 3.011 | Hs.194691 | G protein-coupled receptor, family C, group 5, member A | GPCR5A3 |
| 3.008 | Hs.496622 | plastin 3 (T isoform) | PLS3 |
| 2.666 | Hs.2 13467 | tumor necrosis factor receptor superfamily, | TNFRSF10D |
| | | member 10d, decoy with truncated death domain | |
| 2.523 | Hs.5 15258 | prostate differentiation factor, Growth | GDF15 |
| | | differentiation factor 15 | |
| 0.385 | Hs.1634 | cell division cycle 25A | CDC25A |

NOTE:
Three independent sets of total RNA were analyzed by oligonucleotide microarrays. The Fold Change describes the fold difference of the genomic means between hypoxic samples and normoxic samples. The threshold was set to a minimum of 2.5-fold average regulation.

TABLE 6
Target genes of the HIF-1á-Myc pathway

| Gene | Target | | HIF-1á | p53 | Sp1 | Canonical |
|---|---|---|---|---|---|---|
| Expression | Genes | | Sufficiency | Requirement | Involvement | E-box |
| | CDC25A | ↓ | − | − | ? | − |
| | CDKN1A | ↑ | + | − | ? | − |
| | MSH2 | ↓ | + | + | + | − |
| | MSH6 | ↓ | + | + | ? | − |
| | NBS1 | ↓ | + | − | ? | − |

NOTE:
Arrow denotes gene up-regulation or down-regulation, and question mark not yet examined. No canonical Myc-binding E-box has been reported in the region where Myc displacement occurs in these target genes.

### G. References

Acker T, Diez-Juan A, Aragones J, Tjwa M, Brusselmans K, Moons L, Fukumura D, Moreno-Murciano M P, Herbert J M, Burger A, Riedel J, Elvert G, Flamme I, Maxwell P H, Collen D, Dewerchin M, Jain R K, Plate K H, Carmeliet P “Genetic evidence for a tumor suppressor role of HIF-2α.” Cancer Cell 8:131-141 (2005).
Adhikary S, Marinoni F, Hock A, Hulleman E, Popov N, Beier R, Bernard S, Quarto M, Capra M, Goettig S, Kogel U, Scheffner M, Helin K, Eilers M. “The ubiquitin ligase HectH9 regulates transcriptional activation by Myc and is essential for tumor cell proliferation.” Cell 123:409-21 2005.
Barre B, Vigneron A, Coqueret O. “The STAT3 transcription factor is a target for the Myc and riboblastoma proteins on the Cdc25A promoter.” J Biol Chem 280:15673-81 (2005).
Bartek J, Lukas C, Lukas J. “Checking on DNA damage in S phase.” Nat Rev Mol Cell Biol 5:792-804 (2004).
Bartek J, Lukas J. “Mammalian G1- and S-phase checkpoints in response to DNA damage.” Curr Opin Cell Biol 13:738-47 (2001).
Bindra R S, Gibson S L, Meng A, Westermark U, Jasin M, Pierce A J, Bristow R G, Classon M K, Glazer P M “Hypoxia-induced down-regulation of BRCA1 expression by E2Fs.” Cancer Res 65: 11597-11604 (2005).
Bindra R S “Glazer P M Genetic instability and the tumor microenvironment: towards the concept of microenvironment-induced mutagenesis.” Mutat Res 569: 75-85 (2005).
Bindra R S, Schaffer P J, Meng A, Woo J, Maseide K, Roth M E, Lizardi P, Hedley D W, Bristow R G, Glazer P M “Down-regulation of Rad51 and decreased homologous recombination in hypoxic cancer cells.” Mol Cell Biol 24: 8504-8518 (2004).
Blancher C, Moore J W, Talks K L, Houlbrook S, Harris A L “Relationship of hypoxia-inducible factor (HIF)-1α and HIF-2α expression to vascular endothelial growth factor induction and hypoxia survival in human breast cancer cell lines.” Cancer Res 60: 7106-7113 (2000).
Bunn H F, Poyton R O. “Oxygen sensing and molecular adaptation to hypoxia.” Physiol Rev 76:839-85 (1996).
Carmeliet P, Dor Y, Herbert J M, Fukumura D, Brusselmans K, Dewerchin M, Neeman M, Bono F, Abramovitch R, Maxwell P, Koch C J, Ratcliffe P, Moons L, Jain R K, Collen D, Keshet E “Role of HIF-1α in hypoxia-mediated apoptosis, cell proliferation and tumour angiogenesis.” Nature 394: 485-490 (1998).
Carney J P, Maser R S, Olivares H, Davis E M, Le Beau M, Yates III J R, Hays L, Morgan W F, Petrini J H “The hMre11/hRad50 protein complex and Nijmegen breakage syndrome: linkage of double-strand break repair to the cellular DNA damage response.” Cell 93: 477-486 (1998).
Chen X, Prywes R. “Serum-induced expression of the cdc25A gene by relief of E2F-mediated repression.” Mol Cell Biol 19:4695-702 (1999).
Chiang Y C, Teng S C, Su Y N, Hsieh F J, Wu K J “c-Myc directly regulates the transcription of the NBS1 gene involved in DNA double-strand break repair.” J Biol Chem 278: 19286-19291 (2003).
Compernolle V, Brusselmans K, Acker T, Hoet P, Tjwa M, Beck H, Plaisance S, Dor Y, Keshet E, Lupu F, Nemery B, Dewerchin M, Van Veldhoven P, Plate K, Moons L, Collen D, Carmeliet P “Loss of HIF-2α and inhibition of VEGF impair fetal lung maturation, whereas treatment with VEGF prevents fatal respiratory distress in premature mice.” Nat Med 8: 702-710 (2002).
Coquelle A, Toledo F, Stern S, Bieth A, Debatisse M “A new role for hypoxia in tumor progression: induction of fragile site triggering genomic rearrangements and formation of complex DMs and HSRs.” Mol Cell 2:259-265 (1998).
Covello K L, Kehler J, Yu H, Gordan J D, Arsham A M, Hu C J, Labosky P A, Simon M C, Keith B “HIF-2α regulates Oct-4: effects of hypoxia on stem cell function, embryonic development, and tumor growth.” Genes Dev 20: 557-570 (2006).
Covello K L, Simon M C, Keith B “Targeted replacement of hypoxia-inducible factor-1α by a hypoxia-inducible factor-2α knock-in allele promotes tumor growth.” Cancer Res 65:2277-2286 (2005).
D'Amours D, Jackson S P “The Mre11 complex: at the crossroads of DNA repair and checkpoint signaling.” Nat Rev Mol Cell Biol 3: 317-327 (2002).
Erbel P J, Card P B, Karakuzu O, Bruick R K, Gardner K H “Structural basis for PAS domain heterodimerization in the basic helix-loop-helix-PAS transcription factor hypoxia-inducible factor.” Proc Natl Acad Sci USA 100:15504-15509 (2003).
Frappart P O, Tong W M, Demuth I, Radovanovic I, Herceg Z, Aguzzi A, Digweed M, Wang Z Q “An essential function for NBS1 in the prevention of ataxia and cerebellar defects.” Nat Med 11:538-544 (2005).
Friedberg E C “DNA damage and repair.” Nature 421:436-440 (2003).
Galaktionov K, Chen X, Beach D. “Cdc25 cell-cycle phosphatase as a target of c-myc.” Nature 382:511-7 (1996).
Gardner L B, Li Q, Park M S, Flanagan W M, Semenza G L, Dang C V. “Hypoxia inhibits G1/S transition through regulation of p27 expression.” J Biol Chem 276:7919-26 (2001).
Gartel A L, Radhakrishnan S K. “Lost in transcription: p21 repression, mechanisms, and consequences.” Cancer Res 65:3980-5.
Giaccia A, Siim B G, Johnson R S “HIF-1 as a target for drug development.” Nat Rev Drug Discov 2: 803-811 (2003).
Goda N, Ryan H E, Khadivi B, McNulty W, Rickert R C, Johnson R S “Hypoxia-inducible factor 1α is essential for cell cycle arrest during hypoxia.” Mol Cell Biol 23: 359-369 (2003).
Gu J, Milligan J, Huang L E “Molecular mechanism of hypoxia-inducible factor 1α-p300 interaction. A leucine-rich interface regulated by a single cysteine.” J Biol Chem 276: 3550-3554 (2001).
Gu Y, Rosenblatt J, Morgan D O. “Cell cycle regulation of CDK2 activity by phosphorylation of Thr160 and Tyr15.” Embo 11:3995-4005 J (1992).
Hammond E M, Dorie M J, Giaccia A J “Inhibition of ATR leads to increased sensitivity to hypoxia/reoxygenation.” Cancer Res 64: 6556-6562 (2004).
Hammond E M, Dorie M J, Giaccia A J “ATR/ATM targets are phosphorylated by ATR in response to hypoxia and ATM in response to reoxygenation.” J Biol Chem 278: 12207-12213 (2003).
Hammond E M, Denko N C, Dorie M J, Abraham R T, Giaccia A J “Hypoxia links ATR and p53 through replication arrest.” Mol Cell Biol 22: 1834-1843 (2002).
Hu C J, Wang L Y, Chodosh L A, Keith B, Simon M C “Differential roles of hypoxia-inducible factor 1α (HIF-1α) and HIF-2α in hypoxic gene regulation.” Mol Cell Biol 23: 9361-9374 (2003).
Huang L E “Targeting HIF-α: when a magic arrow hits the bull's eye.” Drug Discov Today 9: 869 (2004).
Huang L E, Bunn H F “Hypoxia-inducible factor and its biomedical relevance.” J Biol Chem 278: 19575-19578 (2003).
Huang L E, Pete E A, Schau M, Milligan J, Gu J “Leu-574 of HIF-1α is essential for the von Hippel-Lindau (VHL)-mediated degradation pathway.” J Biol Chem 277: 41750-41755 (2002).
Huang L E, Gu J, Schau M, Bunn H F “Regulation of hypoxia-inducible factor 1 a is mediated by an O2-dependent degradation domain via the ubiquitin-proteasome pathway.” Proc Natl Acad Sci USA 95: 7987-7992 (1998).
Huang L E, Arany Z, Livingston D M, Bunn H F. “Activation of hypoxia-inducible transcription factor depends primarily upon redox-sensitive stabilization of its alpha subunit.” J Biol Chem 271:32253-9 (1996).
Iavarone A, Massague J. “E2F and histone deacetylase mediate transforming growth factor beta repression of cdc25A during keratinocyte cell cycle arrest.” Mol Cell Biol 1999; 19:916-22.
Jinno S, Suto K, Nagata A, Igarashi M, Kanaoka Y, Nojima H, Okayama H. “Cdc25A is a novel phosphatase functioning early in the cell cycle.” Embo J 13:1549-56 1994).
Kaelin W G “Proline hydroxylation and gene expression.” Annu Rev Biochem 74: 115-128 (2005).
Kageyama Y, Koshiji M, To K K, Tian Y M, Ratcliffe P J, Huang L E. “Leu-574 of human HIF-1alpha is a molecular determinant of prolyl hydroxylation.” Faseb J 18:1028-30 (2004).
Kim J W, Tchernyshyov I, Semenza G L, Dang C V “HIF-1-mediated expression of pyruvate dehydrogenase kinase: a metabolic switch required for cellular adaptation to hypoxia.” Cell Metab 3: 177-185 (2006).
Kittipatarin C, Li W Q, Bulavin D V, Durum S K, Khaled A R. “Cell cycling through Cdc25A: transducer of cytokine proliferative signals.” Cell Cycle 5:907-12 (2006).
Kobayashi J, Tauchi H, Sakamoto S, Nakamura A, Morishima K, Matsuura S, Kobayashi T, Tamai K, Tanimoto K, Komatsu K “NBS1 localizes to gamma-H2AX foci through interaction with the FHA/BRCT domain.” Curr Biol 12: 1846-1851 (2002).
Kondo K, Klco J, Nakamura E, Lechpammer M, Kaelin W G “Inhibition of HIF is necessary for tumor suppression by the von Hippel-Lindau protein.” Cancer Cell 1: 237-246 (2002).
Koshiji M, To K K, Hammer S, Kumamoto K, Harris A L, Modrich P, Huang L E “HIF-1α induces genetic instability by transcriptionally downregulating MutSa expression.” Mol Cell 17: 793-803 (2005).
Koshiji, M., Kageyama, Y., Pete, E. A., Horikawa, I., Barrett, J. C. and Huang, L. E. “HIF-1α induces cell cycle arrest by functionally counteracting Myc.” Embo J, 23, 1949-1956 (2004).
Koshiji M, Kageyama Y, Pete E A, Horikawa I, Barrett J C, Huang L E “HIF-1α induces cell cycle arrest by functionally counteracting Myc.” EMBO J 23: 1949-1956 (2004).
Koshiji M, Huang L E. “Dynamic balancing of the dual nature of HIF-1alpha for cell survival.” Cell Cycle 3:853-4 (2004).
Lengauer C, Kinzler K W, Vogelstein B “Genetic instabilities in human cancers.” Nature 396: 643-649 (1998).
Mack F A, Patel J H, Biju M P, Haase V H, Simon M C “Decreased growth of Vh1−/− fibrosarcomas is associated with elevated levels of cyclin kinase inhibitors p21 and p27.” Mol Cell Biol 25: 4565-4578 (2005).
Mailand N, Falck J, Lukas C, Syljuasen R G, Welcker M, Bartek J, Lukas J. “Rapid destruction of human Cdc25A in response to DNA damage.” Science 288:1425-9 (2000).
Maranchie J K, Vasselli J R, Riss J, Bonifacino J S, Linehan W M, Klausner R D “The contribution of VHL substrate binding and HIF1-α to the phenotype of VHL loss in renal cell carcinoma.” Cancer Cell 1: 247-255 (2002).
Matsuura S, Tauchi H, Nakamura A, Kondo N, Sakamoto S, Endo S, Smeets D, Solder B, Belohradsky B H, Der Kaloustian V M, Oshimura M, Isomura M, Nakamura Y, Komatsu K “Positional cloning of the gene for Nijmegen breakage syndrome.” Nat Genet 19: 179-181 (1998).
Mihaylova V T, Bindra R S, Yuan J, Campisi D, Narayanan L, Jensen R, Giordano F, Johnson R S, Rockwell S, Glazer P M “Decreased expression of the DNA mismatch repair gene M1h1 under hypoxic stress in mammalian cells.” Mol Cell Biol 23: 3265-3273 (2003).
Nelson D A, Tan T T, Rabson A B, Anderson D, Degenhardt K, White E “Hypoxia and defective apoptosis drive genomic instability and tumorigenesis.” Genes Dev 18: 2095-2107 (2004).
Papandreou I, Cairns R A, Fontana L, Lim A L, Denko N C “HIF-1 mediates adaptation to hypoxia by actively downregulating mitochondrial oxygen consumption.” Cell Metab 3: 187-197 (2006).
Paull T T, Rogakou E P, Yamazaki V, Kirchgessner C U, Gellert M, Bonner W M “A critical role for histone H2AX in recruitment of repair factors to nuclear foci after DNA damage.” Curr Biol 10: 886-895 (2000).
Poellinger L, Johnson R S “HIF-1 and hypoxic response: the plot thickens.” Curr Opin Genet Dev 14: 81-85 (2004).
Pugh C W, Ratcliffe P J “Regulation of angiogenesis by hypoxia: role of the HIF system.” Nat Med 9: 677-684 (2003).
Raval R R, Lau K W, Tran M G, Sowter H M, Mandriota S J, Li J L, Pugh C W, Maxwell P H, Harris A L, Ratcliffe P J “Contrasting properties of hypoxia-inducible factor 1 (HIF-1) and HIF-2 in von Hippel-Lindau-associated renal cell carcinoma.” Mol Cell Biol 25: 5675-5686 (2005).
Rogakou E P, Nieves-Neira W, Boon C, Pommier Y, Bonner W M “Initiation of DNA fragmentation during apoptosis induces phosphorylation of H2AX histone at serine 139.” J Biol Chem 275: 9390-9395 (2000).
Rogakou E P, Boon C, Redon C, Bonner W M “Megabase chromatin domains involved in DNA double-strand breaks in vivo.” J Cell Biol 146: 905-916 (1999).
Rogakou E P, Pilch D R, Orr A H, Ivanova V S, Bonner W M “DNA double-stranded breaks induce histone H2AX phosphorylation on serine 139.” J Biol Chem 273: 5858-5868 (1998).
Rother K, Kirschner R, Sanger K, Bohlig L, Mossner J, Engeland K. “p53 downregulates expression of the G(1)/S cell cycle phosphatase Cdc25A.” Oncogene 26:1949-53 (2007).
Rykx A, De Kimpe L, Mikhalap S, Vantus T, Seufferlein T, Vandenheede J R, Van Lint J “Protein kinase D: a family affair.” FEBS Lett 546: 81-86 (2003).
Schultz L B, Chehab N H, Malikzay A, Halazonetis T D “p53 binding protein 1 (53BP1) is an early participant in the cellular response to DNA double-strand breaks.” J Cell Biol 151: 1381-1390 (2000).
Scortegagna M, Ding K, Oktay Y, Gaur A, Thurmond F, Yan L J, Marck B T, Matsumoto A M, Shelton J M, Richardson J A, Bennett M J, Garcia J A “Multiple organ pathology, metabolic abnormalities and impaired homeostasis of reactive oxygen species in Epas1−/− mice.” Nat Genet 35: 331-340 (2003).
Semenza G L “Targeting HIF-1 for cancer therapy.” Nat Rev Cancer 3: 721-732 (2003).
Semenza G L. Regulation of mammalian O2 homeostasis by hypoxia-inducible factor 1. Annu Rev Cell Dev Biol; 15:551-78 (1999).
Storz P, Doppler H, Johannes F J, Toker A “Tyrosine phosphorylation of protein kinase D in the pleckstrin homology domain leads to activation.” J Biol Chem 278: 17969-17976 (2003).
Stracker T H, Carson C T, Weitzman M D “Adenovirus oncoproteins inactivate the Mre11-Rad50-NBS1 DNA repair complex.” Nature 418: 348-352 (2002).
Talks K L, Turley H, Gatter K C, Maxwell P H, Pugh C W, Ratcliffe P J, Harris A L “The expression and distribution of the hypoxia-inducible factors HIF-1α and HIF-2α in normal human tissues, cancers, and tumor-associated macrophages.” Am J Pathol 157: 411-421 (2000).
Tauchi H, Kobayashi J, Morishima K, van Gent D C, Shiraishi T, Verkaik N S, vanHeems D, Ito E, Nakamura A, Sonoda E, Takata M, Takeda S, Matsuura S, Komatsu K “Nbs1 is essential for DNA repair by homologous recombination in higher vertebrate cells.” Nature 420: 93-98 (2002).
Tian H, Hammer R E, Matsumoto A M, Russell D W, McKnight S L “The hypoxia-responsive transcription factor EPAS1 is essential for catecholamine homeostasis and protection against heart failure during embryonic development.” Genes Dev 12: 3320-3324 (1998).
To K K, Sedelnikova O A, Samons M, Bonner W M, Huang L E. “The phosphorylation status of PAS-B distinguishes HIF-1alpha from HIF-2alpha in NBS1 repression.” Embo J 25:4784-94 (2006).
To K K, Koshiji M, Hammer S, Huang L E “Genetic instability: the dark side of the hypoxic response.” Cell Cycle 4: 881-882 (2005).
Varon R, Vissinga C, Platzer M, Cerosaletti K M, Chrzanowska K H, Saar K, Beckmann G, Seemanova E, Cooper P R, Nowak N J, Stumm M, Weemaes C M, Gatti R A, Wilson R K, Digweed M, Rosenthal A, Sperling K, Concannon P, Reis A “Nibrin, a novel DNA double-strand break repair protein, is mutated in Nijmegen breakage syndrome.” Cell 93: 467-476 (1998).
Vigneron A, Cherier J, Barre B, Gamelin E, Coqueret O. “The cell cycle inhibitor p21waf1 binds to the myc and cdc25A promoters upon DNA damage and induces transcriptional repression.” J Biol Chem 281:34742-50 (2006).
Vigo E, Muller H, Prosperini E, Hateboer G, Cartwright P, Moroni M C, Helin K. “CDC25A phosphatase is a target of E2F and is required for efficient E2F-induced S phase.” Mol Cell Biol 19:6379-95 (1999).
Vogelstein B, Kinzler K W “Cancer genes and the pathways they control.” Nat Med 10: 789-799 (2004). Wang V, Davis D A, Haque M, Huang L E, Yarchoan R. “Differential gene up-regulation by hypoxia-inducible factor-1alpha and hypoxia-inducible factor-2alpha in HEK293T cells.” Cancer Res 65:3299-306 (2005).
Wenger R H “Cellular adaptation to hypoxia: O2-sensing protein hydroxylases, hypoxia-inducible transcription factors, and O2-regulated gene expression.” FASEB J 16: 1151-1162 (2002).
Wiesener M S, Turley H, Allen W E, Willam C, Eckardt K U, Talks K L, Wood S M, Gatter K C, Harris A L, Pugh C W, Ratcliffe P J, Maxwell P H “Induction of endothelial PAS domain protein-1 by hypoxia: characterization and comparison with hypoxia-inducible factor-1α.” Blood 92: 2260-2268 (1998).
Yaffe M B, Leparc G G, Lai J, Obata T, Volinia S, Cantley L C “A motif-based profile scanning approach for genome-wide prediction of signaling pathways.” Nat Biotechnol 19: 348-353 (2001).
Zhao H, Watkins J L, Piwnica-Worms H. “Disruption of the checkpoint kinase 1/cell division cycle 25A pathway abrogates ionizing radiation-induced S and G2 checkpoints.” Proc Natl Acad Sci USA 99:14795-800 (2002).
Zhou B B, Elledge S J “The DNA damage response: putting checkpoints in perspective.” Nature 408: 433-439 (2000).

1. A method of reducing tumor growth in a subject, comprising administering to a subject in need thereof an effective amount of an inhibitor of HIF-1α activity.

2. (canceled)

3. The method of claim 1, wherein the tumor growth is reduced by at least by at least 25%, 50%, 75%, 90%, or 95% in the subject treated.

4. The method of claim 1, wherein the inhibitor is an antibody, a small molecule inhibitor, siRNA, shRNA or an antisense polynucleotide.

5. The method of claim 1, wherein the inhibitor is a small molecule inhibitor.

6. The method of claim 4, wherein the small molecule inhibitor is a pro-drug activated under hypoxic conditions.

7. (canceled)

8. The method of claim 4, wherein the antibody specifically binds to HIF-1α.

9. The method of claim 8, wherein the antibody specifically binds to the PAS-B domain of HIF-1α

10. The method of claim 7, wherein the antibody is a monoclonal human or humanized antibody.

11. The method of claim 1, wherein the inhibitor is a nucleic acid targeting HIF-1α mRNA.

12. The method of claim 1, further comprising the step of administering to the subject an anticancer agent.

13. A method of treating metastasis in a subject with cancer in vivo, comprising: administering to a subject in need thereof an effective amount of an inhibitor of lysyl oxidase activity, thereby inhibiting metastasis, in the subject treated.

14-36. (canceled)

37. A method of treating a subject with cancer, comprising administering to the subject an effective amount of an inhibitor of a component of the Myc pathway, along with antiangiogenesis treatment.

38. The method of claim 38, wherein the wherein the component is Sp1.

39. The method of claim 38, wherein the component is HIF-1α.

40-48. (canceled)

49. A method of staging tumor growth or metastasis in a subject, comprising: assessing HIF-1α levels in a tumor of the subject, whereby a change in HIF-1α level in the tumor in comparison with a reference sample indicates the presence of metastatic tumor growth.

50. The method of claim 50, wherein an increase in HIF-1α level in the tumor in comparison with a reference sample indicates the presence or increase of metastatic tumor growth.

51. (canceled)

52. The method of claim 50, wherein the level of HIF-1α is a level of HIF-1α gene expression, or a level of HIF-1α protein.

53. The method of claim 53, wherein the level of HIF-1α protein in the tumor is measured with an antibody to HIF-1α.

54. The method of claim 53, wherein the level of HIF-1α is determined by measuring the level of enzymatic activity of HIF-1α in the tumor of the subject.

55-59. (canceled)

60. A method for increasing the expression in a target cell of a hypoxia-inducible gene, said method comprising the steps of: (a) introducing into said cell an expression vector comprising a nucleic acid molecule encoding a polypeptide comprising PAS-B of a hypoxia inducible factor, wherein the PAS-B comprises at least one mutation which differs from naturally occurring PAS-B of a hypoxia inducible factor; and (b) allowing expression of said protein encoded by said expression vector.

61-71. (canceled)

72. A method for treating an HIF-1-mediated disorder in a subject comprising administering an effective amount of an HIF-1α PAS-B inhibitor or a mutant PAS-B.

73. The method of claim 73, wherein the HIF-1α PAS-B inhibitor or a mutant PAS-B reduces the level of expression of HIF-1α.

74. (canceled)

75. The method of claim 73, wherein the HIF-1 mediated disorder is selected from the group consisting of cancer, chronic obstructive pulmonary disease, and asthma.

76-94. (canceled)
